(19) United States
(12) Patent Application Publication
(10) Pub. No.: US 2025/0000440 A1
(43) Pub. Date: Jan. 2, 2025

(54) METHOD AND SYSTEM FOR DETECTING FOOT AND LOWER LIMB HEALTH CHANGES

(71) Applicant: ORPYX MEDICAL TECHNOLOGIES INC., CALGARY (CA)

(72) Inventors: EMILY MATIJEVICH, CALGARY (CA); EVAN PATRICK MINTY, CALGARY (CA); MARYAM HAJIZADEH, WINDSOR (CA); JULIA BREANNE EVERETT, CALGARY (CA); MADISON MOLLIE GILEWICZ, CALGARY (CA)

(73) Assignee: ORPYX MEDICAL TECHNOLOGIES INC., CALGARY (CA)

(21) Appl. No.: 18/738,200

(22) Filed: Jun. 10, 2024

Related U.S. Application Data
(60) Provisional application No. 63/626,929, filed on Jan. 30, 2024.
(60) Provisional application No. 63/510,929, filed on Jun. 29, 2023.

(51) Int. Cl. A61B 5/00; A61B 5/01; A61B 5/02; A61B 5/0205; A61B 5/103

(52) U.S. Cl. CPC A61B 5/447; A61B 5/015; A61B 5/02007; A61B 5/02055; A61B 5/6807; A61B 5/7275; A61B 5/746; A61B 5/1038

(57) ABSTRACT

A method for detecting foot and lower limb health changes includes collecting a first and second temperature reading at each time point over a time window, from a first and second plantar location on a user's body. The method further includes determining a temperature offset and applying the temperature offset to the first and second series of temperature readings to yield a first and second series of offset temperature data. Irrelevant offset temperature data is identified and removed from the first and second series of offset temperature data to yield a first and second series of processed temperature data. A temperature difference at each time point in the time window is determined by comparing the temperature readings or processed temperature data and a foot health change flag is generated if the average of the temperature differences over the time period exceeds a foot health change threshold.

### CROSS-REFERENCES TO RELATED APPLICATIONS

This application claims the benefit of U.S. Provisional Patent Application No. 63/510,929 filed on Jun. 29, 2023 and of U.S. Provisional Patent Application No. 63/626,929 filed on Jan. 30, 2024, both of which are incorporated herein by reference in their entirety.

### FIELD

This document relates to the detection of foot health changes. More specifically, this document relates to methods and systems for detecting foot health changes from a series of temperature readings collected from an input device worn by a user.

### BACKGROUND

International Patent Application Publication No. WO 2009/005373A1 (Russel), published January 8th 2009, discloses a temperature sensing system for preventing foot ulceration for persons with diabetes for example comprises temperature sensor members integral with, or capable of insertion into footwear for acquiring user foot temperature data, and a module for example in a wrist watch or adapted to be fixed to clothing or a bell or arranged to communicate wirelessly with one or both of the temperature sensor units. The system is arranged to detect and compare differences between the user's feet temperature and output an audible or visual alarm in the event of a difference beyond a threshold to alert the user of a potential adverse health problem.

U.S. Pat. No. 9,095,305B2 (Engler et al.) discloses a method for monitoring a patient's foot and provides an open platform for receiving at least one foot. The platform has at least one temperature sensor for generating a plurality of temperature data values after receipt of the at least one foot. The method then forms a thermogram of the sole of the at least one foot from the temperature data, and determines whether the thermogram presents at least one of a plurality of prescribed patterns. Next, the method produces output information indicating the emergence of a pre-ulcer or progression of a known pre-ulcer in the at least one foot as a function of whether the thermogram is determined to present the at least one pattern.

U.S. Pat. No. 10,602,932B2 (Ma et al.) discloses one variation of a method for detecting inflammation in a foot including: accessing a first temperature measured through a left temperature sensor and a second temperature measured through a right temperature sensor at approximately a first time, the left temperature sensor arranged in a left sock and the right temperature sensor arranged in a right sock worn on the user's feet; calculating a baseline difference between the first and second temperatures based on confirmation of absence of inflammation in the user's left and right feet at the first time; accessing a third temperature measured through the left temperature sensor and a fourth temperature measured through the right temperature sensor at approximately a second time; and in response to a second temperature difference-between the third and fourth temperatures-differing from the baseline difference by more than a threshold difference, issuing an alarm through the user interface.

### SUMMARY

The following summary is intended to introduce the reader to various aspects of the detailed description, but not to define or delimit any invention.

A method for detecting foot health changes is disclosed. According to some aspects, a method for detecting foot health changes includes, at each of a series of time points over a time period, collecting a first temperature reading at a first plantar location on a body of a user, to yield a first series of temperature readings; at each of the series of time points over the time period, collecting a second temperature reading at a second plantar location on the body of the user, to yield a second series of temperature readings; determining a temperature offset; applying the temperature offset to the first series of temperature readings to generate a first series of offset temperature data; applying the temperature offset to the second series of temperature readings to generate a second series of offset temperature data; identifying and removing irrelevant offset temperature data from the first series of offset temperature data to generate a first series of processed temperature data; identifying and removing the irrelevant offset temperature data from the second series of offset temperature data to generate a second series of processed temperature data; comparing the first series of processed temperature data to the second series of processed temperature data, to determine a temperature difference for each time point; and generating a foot health change flag if an average of the temperature differences over the time period exceeds a foot health change threshold.

In some examples, the first series of temperature readings and the second series of temperature readings are collected by an input device, the input device worn on a foot of the user.

In some examples, the input device is footwear.

In some examples, the input device is an insole or a pair of insoles.

In some examples, the foot health change threshold is at least 2 degrees Celsius.

In some examples, the time period is 3 days.

In some examples, the first plantar location and the second plantar location are each located under one of a heel, a first metatarsal, a third metatarsal, a fifth metatarsal, and a hallux.

In some examples, the first plantar location is on a first foot of the user and the second plantar location is on a second foot of the user.

In some examples, the first plantar location and the second plantar location are on a same foot of the user.

In some examples, the second temperature reading at the second plantar location is an average of temperature readings of all or a subset of plantar locations collected by the input device.

In some examples, the foot health change flag includes an alert.

In some examples, the temperature offset for the first series of temperature readings and/or the second series of temperature readings is zero.

In some examples, the temperature offset for the first series of temperature readings and/or the second series of temperature readings is an average of previous temperature readings for the user.

In some examples, the temperature offset for the first series of temperature readings and/or the second series of temperature readings is an average of five days of the previous temperature readings for the user.

In some examples, the previous temperature readings for the user are collected at a set time after a first time point in the series of time points.

In some examples, the set time is 60 minutes after the first time point in the series of the time points.

In some examples, the temperature offset is determined from the first series of temperature readings and/or the second series of temperature readings taken when the input device is not worn by the user. In some examples, the temperature offset is determined from the first series of temperature readings and/or the second series of temperature readings taken when the input device is at a known temperature.

In some examples, the temperature offset is determined from the first series of temperature readings and/or the second series of temperature readings taken from a group of users.

In some examples, the first series of temperature readings and the second series of temperature readings include at least one data session.

In some examples, the at least one data session starts when the user dons the input device and the at least one data session ends when the user doffs the input device.

In some examples, determining the temperature offset includes removing the at least one data session if the at least one data session fails to meet a data integrity criterion; and generating the temperature offset if a minimum number of data sessions is achieved and a calculation period has ended.

In some examples, the calculation period is two weeks.

In some examples, the minimum number of data sessions is 5.

In some examples, the method further includes updating the temperature offset using at least one additional data session collected during an update period.

In some examples, updating the temperature offset includes removing the at least one additional data session if the at least one additional data session fails to meet the data integrity criterion; determining an updated temperature offset if a minimum number of additional data sessions is achieved and the update period has ended; and applying the updated temperature offset when the difference between the temperature offset and the updated temperature offset exceeds an update threshold.

In some examples, the update period is two weeks.

In some examples, the minimum number of additional data sessions is 5.

In some examples, the temperature offset is applied when the difference between the temperature offset and the updated temperature offset fails to exceed the update threshold.

In some examples, the update threshold is 1 degree Celsius.

In some examples, failing to meet the data integrity criterion includes one of detecting wireless charger heat, detecting a data connection issue, and detecting a data session duration below a duration threshold.

In some examples, the irrelevant offset temperature data includes temperature readings that fall below a broken sensor low threshold or that exceed a broken sensor high threshold.

In some examples, the irrelevant offset temperature data includes offset temperature data within an initial wear time window, the initial wear time window occurring within the time period, the initial wear time window starting when the user dons the input device; or offset temperature data within an end of wear time window, the end of wear time window occurring within the time period, the end of wear time window ending when the user doffs the input device.

In some examples, the initial wear time window has a duration of 30 minutes, or the end of wear time window has a duration of 60 minutes.

In some examples, the method further includes calculating a standard deviation of the first series of offset temperature data and/or the second series of offset temperature data over the time period and comparing the standard deviation to a standard deviation threshold, and wherein the irrelevant offset temperature data includes a subset of the first series of offset temperature data and/or the second series of offset temperature data whose standard deviation exceeds the standard deviation threshold.

In some examples, the standard deviation threshold is 10.

In some examples, the irrelevant offset temperature data includes the entirety of the first series of offset temperature data when the standard deviation calculated at any of the time points over the time period of the first series of offset temperature data exceeds the standard deviation threshold; and/or the entirety of the second series of offset temperature data when the standard deviation calculated at any of the time points over the time period of the second series of offset temperature data exceeds the standard deviation threshold.

In some examples, identifying and removing irrelevant offset temperature data from the first series of offset temperature data to generate a first series of processed temperature data includes calculating a first offset temperature difference between a highest temperature value and a lowest temperature value in the first series of offset temperature data over the time period, comparing the first offset temperature difference to a temperature difference threshold, and including the first series of offset temperature data in the irrelevant offset temperature data if the offset temperature difference for the first series of offset temperature data exceeds the temperature difference threshold; and/or identifying and removing irrelevant offset temperature data from the second series of offset temperature data to generate a second series of processed temperature data includes calculating a second offset temperature difference between a highest temperature value and a lowest temperature value in the second series of offset temperature data over the time period, comparing the second offset temperature difference to a temperature difference threshold, and including the second series of offset temperature data in the irrelevant offset temperature data if the second offset temperature difference for the first series of offset temperature data exceeds the temperature difference threshold.

In some examples, the temperature difference threshold is 15 degrees Celsius.

In some examples, the input device is charged by a wireless charger, wherein the wireless charger includes a wireless charger receiver and a wireless charger transmitter, and the wireless charger receiver is disposed within the input device.

In some examples, the irrelevant offset temperature data includes offset temperature data derived from temperature sensors positioned in proximity to the wireless charger receiver, and offset temperature data exceeds a wireless charging temperature threshold.

In some examples, the wireless charging temperature threshold is the mean of the offset temperature data derived from temperature sensors not positioned in proximity to the wireless charger receiver.

In some examples, the first series of temperature readings and/or the second series of temperature readings are not collected when the wireless charger is in use.

In some examples, the method further includes applying a smoothing filter to the first series of temperature data and/or the second series of temperature data.

In some examples, the method further includes generating synthesized temperature data by applying interpolation methods to the first series of temperature data and/or the second series of temperature data.

In some examples, the method further includes identifying and removing the irrelevant offset temperature data from the first series of offset temperature data includes identifying and removing a corresponding irrelevant temperature reading from the first series of temperature readings and identifying and removing irrelevant offset temperature data from the second series of offset temperature data includes identifying and removing the corresponding irrelevant temperature reading from the second series of temperature readings.

A system for detecting foot health changes is disclosed. According to some aspects, a system for detecting foot health changes includes an input device worn on the body of a user, the input device including: a first temperature sensor located at a first plantar location on the body of the user, the first temperature sensor used to collect a first temperature reading at each of a series of time points over a time period to yield a first series of temperature readings; a second temperature sensor located at a second plantar location on the body of the user, the second temperature sensor used to collect a second temperature reading at each of the series of time points over the time period to yield a second series of temperature readings; a processor in communication with the input device, the processor configured to: receive the first series of temperature readings and the second series of temperature readings from the input device; determine a temperature offset; apply the temperature offset to the first series of temperature readings to generate a first series of offset temperature data; apply the temperature offset to the second series of temperature readings to generate a second series of offset temperature data; identify and remove irrelevant offset temperature data from the first series of offset temperature data to generate a first series of processed temperature data; identify and remove the irrelevant offset temperature data from the second series of offset temperature data to generate a second series of processed temperature data; compare the first series of processed temperature data to the second series of processed temperature data, to determine a temperature difference for each time point; and generate a flag foot health change flag if an average of the temperature differences over the time period exceeds a foot health change threshold.

In some examples, the input device is footwear.

In some examples, the input device is an insole or a pair of insoles.

In some examples, the foot health change threshold is at least 2 degrees Celsius. In some examples, the time period is 3 days.

In some examples, the first plantar location and the second plantar location are each located under one of a heel, a first metatarsal, a third metatarsal, a fifth metatarsal, and a hallux.

In some examples, the first plantar location is on a first foot of the user and the second plantar location is on a second foot of the user.

In some examples, the first plantar location and the second plantar location are on a same foot of the user.

In some examples, the second temperature reading at the second plantar location is an average of temperature readings of all or a subset of plantar locations collected by the input device.

In some examples, the foot health change flag includes an alert.

In some examples, the temperature offset for the series of temperature readings is zero.

In some examples, the temperature offset for the first series of temperature readings and/or the second series of temperature readings is zero.

In some examples, the temperature offset for the first series of temperature readings and/or the second series of temperature readings is an average of previous temperature readings for the user.

In some examples, the temperature offset for the first series of temperature readings and/or the second series of temperature readings is an average of five days of the previous temperature readings for the user.

In some examples, the previous temperature readings for the user are collected at a set time after a first time point in the series of time points.

In some examples, the set time is 60 minutes after the first time point in the series of the time points.

In some examples, the temperature offset is determined from the first series of temperature readings and/or the second series of temperature readings taken when the input device is not worn by the user.

In some examples, the temperature offset is determined from the first series of temperature readings and/or the second series of temperature readings taken when the input device is at a known temperature.

In some examples, the temperature offset is determined from the first series of temperature readings and/or the second series of temperature readings taken from a group of users.

In some examples, the first series of temperature readings and the second series of temperature readings include at least one data session.

In some examples, the at least one data session starts when the user dons the input device and the at least one data session ends when the user doffs the input device.

In some examples, determining the temperature offset includes removing the at least one data session if the at least one data session fails to meet a data integrity criterion; and generating the temperature offset if a minimum number of data sessions is achieved and a calculation period has ended.

In some examples, the calculation period is two weeks.

In some examples, the minimum number of data sessions is 5.

In some examples, the processor is further configured to update the temperature offset using at least one additional data session collected during an update period.

In some examples, updating the temperature offset further includes: removing the at least one additional data session if the at least one additional data session fails to meet the data integrity criterion; determining an updated temperature offset if a minimum number of additional data sessions is achieved and the update period has ended; and applying the updated temperature offset when a difference between the temperature offset and the updated temperature offset exceeds a recalculation threshold.

In some examples, the update period is two weeks.

In some examples, the minimum number of additional data sessions is 5.

In some examples, the temperature offset is applied when the difference between the temperature offset and the updated temperature offset fails to exceed the update threshold.

In some examples, the update threshold is 1 degree Celsius.

In some examples, failing to meet the data integrity criterion includes one of detecting wireless charger heat, detecting a data connection issue, and detecting a data session duration below a duration threshold.

In some examples, the irrelevant offset temperature data includes temperature readings that fall below a broken sensor low threshold or that exceed a broken sensor high threshold.

In some examples, the irrelevant offset temperature data includes: offset temperature data within an initial wear time window, the initial wear time window occurring within the time period, the initial wear time window starting when the user dons the input device; or the offset temperature data within an end of wear time window, the end of wear time window occurring within the time period, the end of wear time window ending when the user doffs the input device.

In some examples, the initial wear time window has a duration of 30 minutes, or the end of wear time window has a duration of 60 minutes.

In some examples, the processor is further configured to calculate a standard deviation of the first series of offset temperature data and/or the second series of offset temperature data over the time period and compare the standard deviation to a standard deviation threshold, and wherein the irrelevant offset temperature data includes a subset of the first series of offset temperature data and/or the second series of offset temperature data whose standard deviation exceeds the standard deviation threshold.

In some examples, the standard deviation threshold is 10.

In some examples, the irrelevant offset temperature data includes: the entirety of the first series of offset temperature data when the standard deviation calculated at any of the time points over the time period of the first series of offset temperature data exceeds the standard deviation threshold; and/or the entirety of the second series of offset temperature data when the standard deviation at any of the time points over the time period of the second series of offset temperature data exceeds the standard deviation threshold.

In some examples, identifying and removing irrelevant offset temperature data from the first series of offset temperature data to generate a first series of processed temperature data includes calculating a first offset temperature difference between a highest temperature value and a lowest temperature value in the first series of offset temperature data over the time period, comparing the first offset temperature difference to a temperature difference threshold, and including the first series of offset temperature data in the irrelevant offset temperature data if the offset temperature difference for the first series of offset temperature data exceeds the temperature difference threshold; and/or identifying and removing irrelevant offset temperature data from the second series of offset temperature data to generate a second series of processed temperature data includes calculating a second offset temperature difference between a highest temperature value and a lowest temperature value in the second series of offset temperature data over the time period, comparing the second offset temperature difference to a temperature difference threshold, and including the second series of offset temperature data in the irrelevant offset temperature data if the second offset temperature difference for the first series of offset temperature data exceeds the temperature difference threshold.

In some examples, the temperature difference threshold is 15 degrees Celsius.

In some examples, the input device is charged by a wireless charger, wherein the wireless charger includes a wireless charger receiver and a wireless charger transmitter, and the wireless charger receiver is disposed within the input device.

In some examples, the irrelevant offset temperature data includes a subset of the first series of offset temperature data and/or the second series of offset temperature data derived from temperature sensors positioned in proximity to the wireless charger receiver, and the subset of the first series of offset temperature data and/or the second series of offset temperature data exceeds a wireless charging temperature threshold.

In some examples, the wireless charging temperature threshold is the mean of the subset of the first series of offset temperature data and/or the second series of offset temperature data derived from temperature sensors not positioned in proximity to the wireless charger receiver.

In some examples, wherein the first series of temperature readings and/or the second series of temperature readings are not collected when the wireless charger is in use.

In some examples, the processor is further configured to apply a smoothing filter to the first series of temperature data and/or the second series of temperature data.

In some examples, the processor is further configured to generate synthesized temperature data by applying interpolation methods to the first series of temperature data and/or the second series of temperature data.

In some examples, identifying and removing the irrelevant offset temperature data from the first series of offset temperature data includes identifying and removing a corresponding irrelevant temperature reading from the first series of temperature readings and identifying and removing the irrelevant offset temperature data from the second series of offset temperature data includes identifying and removing the corresponding irrelevant temperature reading from the second series of temperature readings.

A method for detecting foot health changes is disclosed. According to some aspects, a method for detecting foot health changes includes at each of a series of time points over a time period, collecting a first temperature reading at a first plantar location on a body of a user, to yield a first series of temperature readings; at each of the series of time points over the time period, collecting a second temperature reading at a second plantar location on the body of the user, to yield a second series of temperature readings; at each of the series of time points over the time period, collecting a third temperature reading at a third plantar location on the body of the user, to yield a third series of temperature readings, wherein the first plantar location, the second plantar location and the third plantar location are on a first foot of the user; generating an average temperature gradient, the average temperature gradient including an ordered set of an average of the first series of temperature readings, an average of the second series of temperature readings, and an average of the third series of temperature readings; generating a match percentage for each member of the ordered set of the average temperature gradient by comparing each member of the ordered set of the average temperature gradient to each respective member of a foot deformity temperature gradient; and generating a foot health change flag if the match percentage exceeds a percentage threshold.

In some examples, the method further includes generating a flag where a foot deformity is identified.

In some examples, the foot health change flag includes an alert.

In some examples, the method further includes determining a temperature offset and applying the temperature offset to the first series of temperature readings, to the second series of temperature readings, and/or to the third series of temperature readings.

In some examples, the method further includes calculating a personalized temperature offset using the match percentage of each member of the ordered set of the average temperature gradient.

In some examples, each member of the ordered set of the average temperature gradient corresponds to a respective member in the foot deformity temperature gradient, the respective member in the foot deformity temperature gradient located at a same plantar location as the respective member of the ordered set of the average temperature gradient.

In some examples, the first plantar location, the second plantar location, and the third plantar location are each located under one of a heel, a first metatarsal, a third metatarsal, a fifth metatarsal, and a hallux of the user.

In some examples, the average temperature gradient and a location of the foot health change flag are indicated on a thermal image display.

In some examples, the method further includes calculating a custom insole adjustment factor using the percentage match of each member of the ordered set, wherein a custom insole is shaped using the custom insole adjustment factor, and the custom insole is worn by the user to equalize plantar pressures at the foot deformity.

In some examples, the method further includes manufacturing the custom insole when the percentage match exceeds the percentage threshold.

In some examples, the foot deformity is one of Charcot foot, hammer toe, claw toe, mallet toe, flatfoot, high arches, heel spur, splayfoot, pronated foot, equinus foot, partial foot amputation, and bunion.

A system for detecting foot health changes is disclosed. According to some aspects, a system for detecting foot health changes includes an input device worn on the body of a user, the input device including: a first temperature sensor located at a first plantar location on the body of the user, the first temperature sensor used to collect a first temperature reading at each of a series of time points over a time period to yield a first series of temperature readings; a second temperature sensor located at a second plantar location on the body of the user, the second temperature sensor used to collect a second temperature reading at each of a series of time points over a time period to yield a second series of temperature readings; a third temperature sensor located at a third plantar location on the body of the user, the third temperature sensor used to collect a third temperature reading at each of a series of time points over a time period to yield a third series of temperature readings, wherein the first plantar location, the second plantar location and the third plantar location are on a first foot of the user; a processor in communication with the input device, the processor configured to: receive the first series of temperature readings and the second series of temperature readings from the input device; generate an average temperature gradient, the average temperature gradient including an ordered set of an average of the first series of temperature readings, an average of the second series of temperature readings, and an average of the third series of temperature readings; generate a match percentage for each member of the ordered set of the average temperature gradient by comparing each member of the ordered set of the average temperature gradient to each respective member of a foot deformity temperature gradient; and generate a foot health change flag if the match percentage exceeds a percentage threshold.

In some examples, the processor is further configured to generate a flag where a foot deformity is identified.

In some examples, the foot health change flag includes an alert.

In some examples, the processor is further configured to determine a temperature offset and apply the temperature offset to the first series of temperature readings, to the second series of temperature readings, and/or to the third series of temperature readings.

In some examples, the processor is further configured to calculate a personalized temperature offset using the match percentage of each member of the ordered set of the average temperature gradient.

In some examples, each member of the ordered set of the average temperature gradient corresponds to a respective member in the foot deformity temperature gradient, the respective member in the foot deformity temperature gradient located at a same plantar location as the respective member of the ordered set of the average temperature gradient.

In some examples, the first plantar location, the second plantar location, and the third plantar location are each located under one of a heel, a first metatarsal, a third metatarsal, a fifth metatarsal, and a hallux of the user.

In some examples, the average temperature gradient and a location of the foot health change flag are presented on a thermal image display.

In some examples, the processor is further configured to calculate a custom insole adjustment factor using the percentage match of each member of the ordered set, wherein a custom insole is shaped using the custom insole adjustment factor, and the custom insole is worn by the user to equalize plantar pressures at the foot deformity.

In some examples, a custom insole is manufactured when the percentage match exceeds the percentage threshold.

In some examples, the foot deformity is one of Charcot foot, hammer toe, claw toe, mallet toe, flatfoot, high arches, heel spur, splayfoot, pronated foot, equinus foot, partial foot amputation, and bunion.

A method for detecting asymmetrical foot temperature patterns is disclosed. According to some aspects, a method for detecting asymmetrical foot temperature patterns includes: at each of a series of time points over a time period, collecting a first temperature reading at a first plantar location on a body of a user, to yield a first series of temperature readings, wherein the first plantar location is on a first foot of the user; at each of the series of time points over the time period, collecting a second temperature reading at a second plantar location on the body of the user, to yield a second series of temperature readings, wherein the second plantar location is on a second foot of the user; comparing the first series of temperature readings to the second series of temperature readings to determine a temperature difference for each time point and generate a series of temperature differences; generating a diverging asymmetrical foot temperature pattern flag if an average rate of change of the series of temperature differences over the time period exceeds a diverging temperature pattern threshold; and generating a converging asymmetrical foot temperature pattern flag if an average rate of change of the series of temperature differences over the time period falls below a converging temperature pattern threshold.

In some examples, the method further includes: calculating the standard deviation of the series of temperature differences over the time period; calculating a difference between a highest temperature difference value and a lowest temperature difference value of the series of temperature differences over the time period; and generating a general asymmetrical foot temperature pattern flag if: the standard deviation of the series of temperature differences exceeds a standard deviation threshold; and/or the difference between the highest temperature difference value and the lowest temperature difference value of the series of temperature differences exceeds a temperature difference threshold.

A method for detecting a foot health change is disclosed. According to some aspects, a method for detecting a foot health changes includes: at each of a series of time points over a first time period, collecting a first temperature reading at a first plantar location on a body of the user, to yield a first series of temperature readings; at each of a series of time points over a second time period, collecting a second temperature reading at the first plantar location on the body of a user, to yield a second series of temperature readings; at each of the series of time points over the first time period, collecting a third temperature reading at a second plantar location on the body of the user, to yield a third series of temperature readings; at each of the series of time points over the second time period, collecting a fourth temperature reading at the second plantar location on the body of the user, to yield a fourth series of temperature readings; comparing the first series of temperature readings to the third series of temperature readings, to determine a temperature difference for each time point in the first time period; generating a first foot health change flag if an average of the temperature differences over the first time period exceeds a first foot health change threshold; comparing the second series of temperature readings to the fourth series of temperature readings, to determine a temperature difference for each time point in the second time period; and generating a second foot health change flag if an average of the temperature differences over the second time period exceeds a second foot health change threshold.

In some examples, the first foot health change flag and the second foot health change flag each correspond to a risk level.

In some examples, the risk level corresponds to a likelihood of developing the foot health change.

In some examples, the risk level is a quantitative risk level or a qualitative risk level. In some examples, the quantitative risk level is 1, 2, 3, or 4.

In some examples, the qualitative risk level is low risk, medium risk, or high risk.

In some examples, the risk level corresponding to the first foot health change flag and the risk level corresponding to the second foot health change flag are the same.

In some examples, the first time period and the second time period are the same duration, the first foot health change threshold is larger than the second foot health change threshold, and the first foot health change flag corresponds to a higher risk level than the second foot health change flag.

In some examples, the first time period is longer in duration than the second time period, the first foot health change threshold and the second foot health change threshold are the same value, and the first foot health change flag corresponds to a higher risk level than the second foot health change flag.

In some examples, the first time period is longer in duration than the second time period, the first foot health change threshold is larger than the second foot health change threshold, and the first foot health change flag corresponds to a highest risk level.

In some examples, the first time period is at least three days and the second time period is at least one day.

In some examples, the first foot health change threshold is 2.2 degrees Celsius, and the second foot health change threshold is 2.0 degrees Celsius.

In some examples, the foot health change is one of a diabetic foot ulcer, foot deformity, a foot infection, microvascular disease, and macrovascular disease.

In some examples, the method further includes determining a temperature offset, and applying the temperature offset to the first series of temperature readings, to the second series of temperature readings, to the third series of temperature readings, and to the fourth series of temperature readings.

In some examples, the first plantar location and the second plantar location are on a same foot of the user.

A system for detecting foot health changes is disclosed. According to some aspects, a system for detecting foot health changes includes: an input device worn on the body of a user, the input device including: a first temperature sensor located at a first plantar location on the body of the user, the first temperature sensor used to collect: a first temperature reading at each of a series of time points over a first time period to yield a first series of temperature readings; a second temperature reading at each of a series of time points over a second time period to yield a second series of temperature readings; a second temperature sensor located at a second plantar location on the body of the user, the second temperature sensor used to collect: a third temperature reading at each of the series of time points over the first time period to yield a third series of temperature readings; a fourth temperature reading at each of the series of time points over the second time period to yield a fourth series of temperature readings; a processor in communication with the input device, the processor configured to: receive the first series of temperature readings and the second series of temperature readings from the input device; compare the first series of temperature readings to the third series of temperature readings, to determine a temperature difference for each time point in the first time period; generate a first foot health change flag if an average of the temperature differences over the first time period exceeds a first foot health change threshold; compare the second series of temperature readings to the fourth series of temperature readings, to determine a temperature difference for each time point in the second time period; and generate a second foot health change flag if an average of the temperature differences over the second time period exceeds a second foot health change threshold.

In some examples, each of the first foot health change flag and the second foot health change flag correspond to a risk level.

In some examples, the risk level corresponds to a likelihood of developing the foot health change.

In some examples, the risk level is a quantitative risk level or a qualitative risk level. In some examples, the quantitative risk level is 1, 2, 3, or 4.

In some examples, the qualitative risk level is low risk, medium risk, or high risk.

In some examples, the risk level corresponding to the first foot health change flag and the risk level corresponding to the second foot health change flag are the same.

In some examples, the first time period and the second time period are the same duration, the first foot health change threshold is larger than the second foot health change threshold, and the first foot health change flag corresponds to a higher risk level than the second foot health change flag.

In some examples, the first time period is longer in duration than the second time period, the first foot health change threshold and the second foot health change threshold are the same value, and the first foot health change flag corresponds to a higher risk level than the second foot health change flag.

In some examples, the first time period is longer in duration than the second time period, the first foot health change threshold is larger than the second foot health change threshold, and the first foot health change flag corresponds to a highest risk level.

In some examples, the first time period is at least three days and the second time period is at least one day.

In some examples, the first foot health change threshold is 2.2 degrees Celsius, and the second foot health change threshold is 2.0 degrees Celsius.

In some examples, the foot health change is one of a diabetic foot ulcer, a foot deformity, a foot infection, microvascular disease, and macrovascular disease.

In some examples, the processor is further configured to determine a temperature offset, and apply the temperature offset to the first series of temperature readings, to the second series of temperature readings, to the third series of temperature readings, and to the fourth series of temperature readings.

In some examples, the first plantar location and the second plantar location are on a same foot of the user.

A method for detecting a vascular concern is disclosed. According to some aspects, a method for detecting a vascular concern includes: at each of a series of time points over a time period, collecting a first temperature reading at a first plantar location on a body of a user, to yield a first series of temperature readings; at each of the series of time points over the time period, collecting a second temperature reading at a second plantar location on the body of the user, to yield a second series of temperature readings, the first series of temperature readings and the second series of temperature readings collected by an input device, the input device worn on a foot of the user; comparing the first series of temperature readings to the second series of temperature readings to determine a temperature difference for each time point and generating a series of temperature differences; generating a magnitude of asymmetry from the series of temperature differences; and generating a vascular concern flag if the magnitude of asymmetry from the series of temperature differences exceeds a vascular concern threshold.

In some examples, the first plantar location on the body of the user and the second plantar location on the body of the user are on a same foot of the user.

In some examples, the first plantar location on the body of the user corresponds to a first angiosome of the user and the second plantar location on the body of the user corresponds to a second angiosome of the user.

In some examples, generating the magnitude of asymmetry from the series of temperature differences includes determining a measure of central tendency of the temperature differences of the series of temperature differences.

In some examples, the vascular concern flag corresponds to a risk level.

In some examples, the risk level is a quantitative risk level or a qualitative risk level. In some examples, the quantitative risk level is 1, 2, 3, or 4.

In some examples, the qualitative risk level is low risk, medium risk, or high risk. In some examples, the input device is footwear.

In some examples, the input device is an insole or a pair of insoles.

In some examples, the method further includes monitoring at least one of posture, activity, or movement of the user.

In some examples, monitoring at least one of posture, activity, or movement of the user includes collecting pressure or motion measurements at a location on the body of the user.

In some examples, collecting the pressure measurements includes at least one pressure sensor disposed in footwear or an insole of the user.

In some examples, collecting the motion measurements includes at least one accelerometer or gyroscope disposed in footwear or an insole of the user.

In some examples, the time period is greater than 24 hours.

In some examples, the vascular concern threshold is at least 1.0 degree Celsius.

In some examples, the method further includes: determining a temperature offset; applying the temperature offset to the first series of temperature readings to generate a first series of offset temperature data; applying the temperature offset to the second series of temperature readings to generate a second series of offset temperature data; and identifying and removing irrelevant temperature readings from the first or second series of temperature readings or identifying and removing irrelevant offset temperature data from the first or second series of offset temperature data.

In some examples, collecting temperature readings includes a plurality of the time periods.

A system for detecting a vascular concern is disclosed. According to some aspects, the system for detecting a vascular concern includes: an input device worn on a foot of a user, the input device including: a first temperature sensor located at a first plantar location on a body of the user, the first temperature sensor used to collect a first temperature reading at each of a series of time points over a time period to yield a first series of temperature readings; a second temperature sensor located at a second plantar location on the body of the user, the second temperature sensor used to collect a second temperature reading at each of the series of time points over the time period to yield a second series of temperature readings; a processor in communication with the input device, the processor configured to: receive the first series of temperature readings and the second series of temperature readings from the input device; compare the first series of temperature readings to the second series of temperature readings to determine a temperature difference for each time point and generating a series of temperature differences; generate a magnitude of asymmetry from the series of temperature differences; and generate a vascular concern flag if the magnitude of asymmetry from the series of temperature differences exceeds a vascular concern threshold.

In some examples, the first plantar location and the second plantar location are each located under one of a heel, a first metatarsal, a third metatarsal, a fifth metatarsal, and a hallux.

In some examples, the first plantar location is on a first foot of the user and the second plantar location is on a second foot of the user.

In some examples, first plantar location and the second plantar location are on a same foot of the user.

In some examples, the second temperature reading at the second plantar location is an average of temperature readings of all or a subset of plantar locations collected by the input device.

In some examples, the vascular concern flag includes an alert.

In some examples, the processor is further configured to: determine a temperature offset; apply the temperature offset to the first series of temperature readings to generate a first series of offset temperature data; apply the temperature offset to the second series of temperature readings to generate a second series of offset temperature data; and identify and remove irrelevant temperature readings from the first or second series of temperature readings or identifying and removing irrelevant offset temperature data from the first or second series of offset temperature data.

In some examples, the temperature offset for the first series of temperature readings and/or the second series of temperature readings is zero.

In some examples, the temperature offset for the first series of temperature readings and/or the second series of temperature readings is an average of previous temperature readings for the user.

In some examples, the temperature offset is determined from the first series of temperature readings and/or the second series of temperature readings taken when the input device is not worn by the user.

In some examples, the temperature offset is determined from the first series of temperature readings and/or the second series of temperature readings taken when the input device is at a known temperature.

In some examples, the first series of temperature readings and the second series of temperature readings include at least one data session.

In some examples, the at least one data session starts when the user dons the input device and the at least one data session ends when the user doffs the input device.

In some examples, determining the temperature offset includes: removing the at least one data session if the at least one data session fails to meet a data integrity criterion; and generating the temperature offset if a minimum number of data sessions is achieved and a calculation period has ended.

In some examples, the input device is charged by a wireless charger, wherein the wireless charger includes a wireless charger receiver and a wireless charger transmitter, and the wireless charger receiver is disposed within the input device.

In some examples, the irrelevant offset temperature data includes offset temperature data derived from temperature sensors positioned in proximity to the wireless charger receiver, and the offset temperature data exceeds a wireless charging temperature threshold.

In some examples, the first series of temperature readings and/or the second series of temperature readings are not collected when the wireless charger is in use.

In some examples, the input device is footwear.

In some examples, the input device is an insole or a pair of insoles.

In some examples, the first plantar location on the body of the user corresponds to a first angiosome of the user and the second plantar location on the body of the user corresponds to a second angiosome of the user.

In some examples, the processor is further configured to monitor at least one of posture, activity, or movement of the user.

In some examples, the system includes at least one pressure sensor disposed in footwear or an insole of the user.

In some examples, the system includes at least one accelerometer disposed in footwear or an insole of the user.

In some examples, the time period is greater than 24 hours.

In some examples, the vascular concern threshold is at least 0.1 degree Celsius.

A method for detecting a vascular asymmetry status is disclosed. According to some aspects, a method for detecting a vascular asymmetry status includes: at each of a series of time points over a time period, collecting a first temperature reading at a first plantar location on a body of a user, to yield a first series of temperature readings; at each of the series of time points over the time period, collecting a second temperature reading at a second plantar location on the body of the user, to yield a second series of temperature readings, the first series of temperature readings and the second series of temperature readings collected by an input device, the input device worn on a foot of the user; comparing the first series of temperature readings to the second series of temperature readings to determine a temperature difference for each time point and generate a series of temperature differences; generating a magnitude of asymmetry from the series of temperature differences; generating an asymmetry trend over the time period from the series of temperature differences; and generating a vascular asymmetry status flag if the magnitude of asymmetry from the series of temperature differences exceeds a vascular change threshold and if the asymmetry trend over the time period exceeds an asymmetry change threshold.

In some examples, the method further includes generating a vascular asymmetry status flag if the magnitude of asymmetry from the series of temperature differences exceeds a vascular change threshold and if the asymmetry trend over the time period meets or falls below an asymmetry change threshold.

In some examples, the first plantar location on the body of the user and the second plantar location on the body of the user are on a same foot of the user.

In some examples, the first plantar location is on a first foot of the user and the second plantar location is on a second foot of the user.

In some examples, the first plantar location on the body of the user corresponds to a first angiosome of the user and the second plantar location on the body of the user corresponds to a second angiosome of the user.

In some examples, generating a magnitude of asymmetry from the series of temperature differences includes determining a measure of central tendency of the temperature differences of the series of temperature differences.

In some examples, the vascular asymmetry flag corresponds to a vascular intervention effectiveness.

In some examples, the vascular asymmetry flag corresponds to the vascular status improving, staying the same, or degrading.

In some examples, the input device is footwear.

In some examples, the input device is an insole or a pair of insoles.

In some examples, the method further includes monitoring at least one of posture, activity, or movement of the user.

In some examples, monitoring at least one of posture, activity, or movement of the user includes collecting pressure measurements or motion measurements at a location on the body of the user.

In some examples, collecting the pressure measurements includes at least one pressure sensor disposed in footwear or an insole of the user.

In some examples, collecting the motion measurements includes at least one accelerometer disposed in footwear or an insole of the user.

In some examples, the time period is greater than 24 hours.

In some examples, the vascular concern threshold is at least 1.0 degree Celsius.

In some examples, the method further includes: determining a temperature offset; applying the temperature offset to the first series of temperature readings to generate a first series of offset temperature data; applying the temperature offset to the second series of temperature readings to generate a second series of offset temperature data; and identifying and removing irrelevant temperature readings from the first or second series of temperature readings or identifying and removing irrelevant offset temperature data from the first or second series of offset temperature data.

In some examples, generating the asymmetry trend over the time period from the series of temperature differences over the time period includes determining a rate of change of the temperature differences over the time period.

In some examples, generating the asymmetry trend over the time period from the series of temperature differences over the time period includes determining a weighted average of the temperature differences over the time period, wherein the temperature readings are weighted by user behavior.

In some examples, the asymmetry trend over the time period from the series of temperature differences over the time period includes determining a modelled linear or non-linear fit of the temperature differences over the time period.

A system for detecting a vascular asymmetry status is disclosed. According to some aspects, a system for detecting a vascular asymmetry status includes: an input device worn on a foot of a user, the input device including: a first temperature sensor located at a first plantar location on the body of the user, the first temperature sensor used to collect a first temperature reading at each of a series of time points over a time period to yield a first series of temperature readings; a second temperature sensor located at a second plantar location on the body of the user, the second temperature sensor used to collect a second temperature reading at each of the series of time points over the time period to yield a second series of temperature readings; a processor in communication with the input device, the processor configured to: compare the first series of temperature readings to the second series of temperature readings to determine a temperature difference for each time point and generate a series of temperature differences; generate a magnitude of asymmetry from the series of temperature differences; generate an asymmetry trend over the time period from the series of temperature differences; and generate a vascular asymmetry status flag if the magnitude of asymmetry from the series of temperature differences exceeds a vascular change threshold and if the asymmetry trend over the time period exceeds an asymmetry change threshold.

In some examples, the processor is configured to generate a vascular asymmetry status flag if the magnitude of asymmetry from the series of temperature differences exceeds a vascular change threshold and if the asymmetry trend over the time period meets or falls below an asymmetry change threshold.

In some examples, the first plantar location and the second plantar location are each located under one of a heel, a first metatarsal, a third metatarsal, a fifth metatarsal, and a hallux.

In some examples, the first plantar location is on a first foot of the user and the second plantar location is on a second foot of the user.

In some examples, the first plantar location and the second plantar location are on a same foot of the user.

In some examples, wherein the second temperature reading at the second plantar location is an average of temperature readings of all or a subset of plantar locations collected by the input device.

In some examples, wherein the vascular asymmetry status flag includes an alert.

In some examples, the processor is further configured to: determine a temperature offset; apply the temperature offset to the first series of temperature readings to generate a first series of offset temperature data; apply the temperature offset to the second series of temperature readings to generate a second series of offset temperature data; and identify and remove irrelevant temperature readings from the first or second series of temperature readings or identifying and removing irrelevant offset temperature data from the first or second series of offset temperature data.

In some examples, the temperature offset for the first series of temperature readings and/or the second series of temperature readings is zero.

In some examples, the temperature offset for the first series of temperature readings and/or the second series of temperature readings is an average of previous temperature readings for the user.

In some examples, the temperature offset is determined from the first series of temperature readings and/or the second series of temperature readings taken when the input device is not worn by the user.

In some examples, the temperature offset is determined from the first series of temperature readings and/or the second series of temperature readings taken when the input device is at a known temperature.

In some examples, the first series of temperature readings and the second series of temperature readings include at least one data session.

In some examples, the at least one data session starts when the user dons the input device and the at least one data session ends when the user doffs the input device.

In some examples, the temperature offset includes: removing the at least one data session if the at least one data session fails to meet a data integrity criterion; and generating the temperature offset if a minimum number of data sessions is achieved and a calculation period has ended.

In some examples, the input device is charged by a wireless charger, wherein the wireless charger includes a wireless charger receiver and a wireless charger transmitter, and the wireless charger receiver is disposed within the input device.

In some examples, the irrelevant offset temperature data includes offset temperature data derived from temperature sensors positioned in proximity to the wireless charger receiver, and the offset temperature data exceeds a wireless charging temperature threshold.

In some examples, the first series of temperature readings and/or the second series of temperature readings are not collected when the wireless charger is in use.

In some examples, the input device is footwear.

In some examples, the input device is an insole or a pair of insoles.

In some examples, the first plantar location on the body of the user corresponds to a first angiosome of the user and the second plantar location on the body of the user corresponds to a second angiosome of the user.

In some examples, the processor is further configured to monitor at least one of posture, activity, or movement of the user.

In some examples, the system includes at least one pressure sensor disposed in footwear or an insole of the user.

In some examples, the system includes at least one accelerometer disposed in footwear or an insole of the user.

In some examples, the time period is greater than 24 hours.

In some examples, the vascular concern threshold is at least 0.1 degree Celsius.

A method for detecting a vascular concern is disclosed. According to some aspects, a method for detecting a vascular concern includes: at each of a series of time points over a time period, collecting a temperature reading at a plantar location on a body of a user, to yield a series of temperature readings, the series of temperature readings collected by an input device, the input device worn on a foot of the user; generating a temperature trend over the time period from the series of temperature readings; and generating a vascular concern status flag if the temperature trend over the time period exceeds a cooling rate threshold.

In some examples, the method further includes generating a magnitude of temperature change from the series of temperature readings.

In some examples, the method further includes generating a vascular concern status flag if the magnitude of temperature change from the series of temperature readings exceeds a vascular concern threshold.

In some examples, the vascular concern threshold is at least 0.01 degree Celsius.

In some examples, the method further includes generating a vascular concern flag if the temperature trend over the time period meets or falls below a cooling rate threshold.

In some examples, the method further includes generating a vascular concern flag if the temperature trend over the time period exceeds, meets, or falls below a cooling rate threshold and the temperature trend is statistically significant.

In some examples, the vascular concern status flag corresponds to a risk level.

In some examples, the risk level is a quantitative risk level or a qualitative risk level.

In some examples, the quantitative risk level is 1, 2, 3, or 4.

In some examples, the qualitative risk level is low risk, medium risk, or high risk.

In some examples, the vascular concern status flag corresponds to one of a cooling vascular concern, a no change in vascular concern, and a warming vascular concern.

In some examples, the cooling rate threshold is zero and the vascular concern status flag corresponds to the no change in vascular concern.

In some examples, the cooling rate threshold is non-zero and the vascular concern status flag indicates a cooling vascular concern.

In some examples, generating the magnitude of temperature change includes determining a difference of a first temperature reading and a second temperature reading from the series of temperature readings.

In some examples, generating the magnitude of temperature change includes determining the difference of the outer values of a 95% confidence interval of the series of temperature readings.

In some examples, the method further includes generating an alert.

In some examples, the input device is footwear.

In some examples, the input device is an insole or a pair of insoles.

In some examples, the method further includes monitoring at least one of posture, activity, or movement of the user.

In some examples, monitoring at least one of posture, activity, or movement of the user includes collecting pressure measurements or motion measurements at a location on the body of the user.

In some examples, collecting the pressure measurements includes at least one pressure sensor disposed in the input device or a wearable item worn by the user.

In some examples, collecting the motion measurements includes at least one accelerometer disposed in the input device or a wearable item worn by the user.

In some examples, the time period is minutes or up to over a year in duration.

In some examples, the method further includes: determining a temperature offset; applying the temperature offset to the series of temperature readings to generate a series of offset temperature data; and identifying and removing irrelevant temperature readings from the series of temperature readings or identifying and removing irrelevant offset temperature data from the series of offset temperature data.

In some examples, generating the temperature trend over the time period from the series of temperature readings over the time period includes determining a rate of change of the series of temperature readings over the time period.

In some examples, generating the temperature trend over the time period from the series of temperature readings over the time period includes determining a weighted average of the temperature readings over the time period, wherein the series of temperature readings are weighted by user behavior.

In some examples, generating the temperature trend over the time period from the series of temperature readings over the time period includes determining a modelled linear or non-linear fit of the series of temperature readings over the time period.

A system for detecting a vascular concern is disclosed. According to some aspects, a system for detecting a vascular concern includes: an input device worn on a foot of a user, the input device including: a temperature sensor located at a plantar location on the body of the user, the temperature sensor used to collect a temperature reading at each of a series of time points over a time period to yield a series of temperature readings; a processor in communication with the input device, the processor configured to: generate a temperature trend from the series of temperature readings; and generate a vascular concern status flag if the temperature trend from the series of temperature readings exceeds a cooling rate threshold.

In some examples, the system further includes generating a magnitude of temperature change from the series of temperature readings.

In some examples, the system further includes generating a vascular concern status flag if the magnitude of temperature change from the series of temperature readings exceeds a vascular concern threshold.

In some examples, the vascular concern threshold is at least 0.01 degree Celsius.

In some examples, the processor is configured to generate a vascular concern status flag if the temperature trend from the series of temperature readings meets or falls below a cooling rate threshold.

In some examples, the plantar location is located under one of a heel, a first metatarsal, a third metatarsal, a fifth metatarsal, and a hallux.

In some examples, the plantar location is on a foot of the user.

In some examples, the temperature reading at the plantar location is an average of temperature readings at a plurality of plantar locations collected by the input device. In some examples, the vascular concern status flag includes an alert.

In some examples, the processor is further configured to: determine a temperature offset; apply the temperature offset to the series of temperature readings to generate a series of offset temperature data; identify and remove irrelevant temperature readings from the series of temperature readings or identifying and removing irrelevant offset temperature data from the series of offset temperature data.

In some examples, the temperature offset for the series of temperature readings is zero.

In some examples, the temperature offset for the series of temperature readings is an average of previous temperature readings for the user.

In some examples, the temperature offset is determined from temperature readings taken when the input device is not worn by the user.

In some examples, the temperature offset is determined from temperature readings taken when the input device is at a known temperature.

In some examples, the series of temperature readings include at least one data session.

In some examples, the at least one data session starts when the user dons the input device and the at least one data session ends when the user doffs the input device.

In some examples, the temperature offset includes: removing the at least one data session if the at least one data session fails to meet a data integrity criterion; and generating the temperature offset if a minimum number of data sessions is achieved and a calculation period has ended.

In some examples, the input device is charged by a wireless charger, wherein the wireless charger includes a wireless charger receiver and a wireless charger transmitter, and the wireless charger receiver is disposed within the input device.

In some examples, the irrelevant offset temperature data includes offset temperature data derived from temperature sensors positioned in proximity to the wireless charger receiver, and the offset temperature data exceeds a wireless charging temperature threshold.

In some examples, temperature readings are not collected when the wireless charger is in use.

In some examples, the input device is footwear.

In some examples, the input device is an insole or a pair of insoles.

In some examples, the processor is further configured to monitor at least one of posture, activity, or movement of the user.

In some examples, the system includes at least one pressure sensor disposed in the input device or a wearable item worn by the user.

In some examples, the system includes at least one accelerometer disposed in the input device or a wearable item worn by the user.

In some examples, the time period is greater than 24 hours.

In some examples, the cooling rate threshold is at least 0.01 degree Celsius per day.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an example input device usable in the methods and systems described herein;

FIG. 2 is an exploded view of the input device of FIG. 1;

FIG. 3 is a flow chart illustrating a method for detecting a foot health change of a user, which may be carried out using the input device of FIG. 1;

FIG. 4 is a flow chart illustrating a method for detecting a foot deformity foot health change of a user, which may be carried out using the input device of FIG. 1;

FIG. 5 is a flow chart illustrating a method for detecting an asymmetrical foot temperature pattern foot health change of a user, which may be carried out using the input device of FIG. 1;

FIG. 6 is a table illustrating an example risk level categorization of the types of foot health change flags;

FIG. 7 is a flow chart illustrating a method for detecting a foot health change and determining the corresponding risk level of the foot health change flag of FIG. 6;

FIG. 8 is a flow chart illustrating a method for determining the temperature offset;

FIG. 9 is a flow chart illustrating a method for updating the temperature offset of FIG. 8;

FIG. 10 is a diagram illustrating an example of the temperature offsets to be applied;

FIG. 11 is a graph illustrating an example set of temperature readings from temperature sensors under the left and right center metatarsal joint of the user;

FIG. 12 is a graph illustrating an example contralateral temperature difference calculation from the set of temperature readings of FIG. 11;

FIG. 13 is a diagram illustrating an example of the filters for removing the irrelevant offset temperature data from the offset temperature data;

FIG. 14 is a graph illustrating an example set of data from a left and right temperature sensor under the heel of the user, showing the application of the loss of contact filter of FIG. 13;

FIG. 15 is a graph illustrating an example set of data from temperature sensors under the left and right heel of the user and after the user has donned the input device, showing the application of the warm up and cool down filter of FIG. 13;

FIG. 16 is a graph illustrating an example set of data from temperature sensors under the left and right heel of the user and after the user has doffed the input device, showing the application of the warm up and cool down filter of FIG. 13;

FIG. 17 is a graph illustrating an example set of data from temperature sensors under the left and right heel of the user and after the user has doffed the input device for wireless charging, showing the application of the warm up and cool down filter of FIG. 13;

FIG. 18 is a flow chart illustrating a method for detecting a vascular concern of a user, which may be carried out using the input device of FIG. 1; and

FIG. 19 is a flow chart illustrating a method for detecting a vascular concern status of a user, which may be carried out using the input device of FIG. 1.

FIG. 20 is a flow chart illustrating a method for detecting a vascular concern status of a user, which may be carried out using the input device of FIG. 1.

### DETAILED DESCRIPTION

Various apparatuses or processes or compositions will be described below to provide an example of an embodiment of the claimed user matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any user matter described below and for which an exclusive right is not granted by issuance of this patent application may be the user matter of another protective instrument, for example, a continuing patent application, and the applicants, inventors or owners do not intend to abandon, disclaim or dedicate to the public any such user matter by its disclosure in this document.

For simplicity and clarity of illustration, reference numerals may be repeated among the figures to indicate corresponding or analogous elements.

Numerous specific details are set forth herein in order to provide a thorough understanding of the user matter described herein. However, the user matter described herein may be practiced without these specific details. In other instances, well-known methods, procedures and components have not been described in detail so as not to obscure the user matter described herein. The description is not to be considered as limiting the scope of the user matter described herein.

The terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices can be directly connected to one another or connected to one another through one or more intermediate elements or devices via an electrical element, electrical signal, or a mechanical element depending on the particular context. Furthermore, the term “communicative coupling” may be used to indicate that an element or device can electrically, optically, or wirelessly send data to another element or device as well as receive data from another element or device.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. Furthermore, the wording “at least one of A and B” is intended to mean only A (i.e. one or multiple of A), only B (i.e. one or multiple of B), or a combination of one or more of A and one or more of B.

Terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

As will be described in further detail below, described herein are methods and systems for detecting foot health changes. As used herein, the term “worn” (and related terms such as “wearable” and “wear”) indicates that the referenced part may be affixed to, adhered to, placed on, or placed in the user's body, or affixed to, adhered to, placed on, placed in, placed in proximity to, or integral with the user's clothing. Such clothing may include but is not limited to a shoe, a sock, an insole, and/or another type of footwear clothing (including custom orthotics or generic insoles).

The sensors described herein can include pressure sensors. As used herein, the term “pressure” is used broadly and can refer to raw force (i.e. with units of N), or pressure resulting from a raw force (i.e. with units of N/m2). The pressure data acquired by the pressure sensors can be used to determine the level of pressure applied by an individual's foot when performing various activities such as walking, running, sliding, or jumping for example. The pressure data acquired can also be used to identify changes in an individual's posture, movement pattern, and/or behavior. In addition, the pressure data acquired can be used to approximate what anatomy lies over a region of the insoles (e.g. to confirm according to the pressure pattern that a certain region of the insole corresponds to the metatarsals, or to correlate the pressure acquired to angiosome locations).

The systems, and methods described herein may be implemented as a combination of hardware or software. In some cases, the systems, methods, and devices described herein may be implemented, at least in part, by using one or more computer programs, executing on one or more programmable devices including at least one processing element, and a data storage element (including volatile and non-volatile memory and/or storage elements). These devices may also have at least one input device (e.g. a pushbutton keyboard, mouse, a touchscreen, and the like), and at least one output device (e.g. a display screen, a printer, a wireless radio, and the like) depending on the nature of the device.

Some elements that are used to implement at least part of the systems, methods, and devices described herein may be implemented via software that is written in a high-level procedural language such as object-oriented programming. Accordingly, the program code may be written in any suitable programming language such as Python or C for example. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device.

The software program code, when read by the programmable device, configures the programmable device to operate in a new, specific and predefined manner in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

Generally disclosed herein are systems and methods for detecting foot health changes including systemic circulatory or blood flow complications. The system includes sensors, which may include a plurality of temperature sensors, which are used to obtain temperature readings, respectively. In particular, the plurality of temperature sensors may be worn on a user's foot over a time period and may be used to obtain a series of temperature readings. In some examples, the temperature sensors are included in an input device in the form of an insole (or alternatively, a pair of insoles) which may be inserted into a user's shoe and worn on the foot. The input device may in turn be part of a system, which may further include a non-transitory storage memory storing algorithms trained to detect foot health changes using at least the series of temperature data, a processor configured to input the series of temperature readings into the algorithms (either as raw measurements or processed measurements), and an output device for outputting an indication of the complication foot health change (e.g. outputting a visual, auditory, or tactile alert).

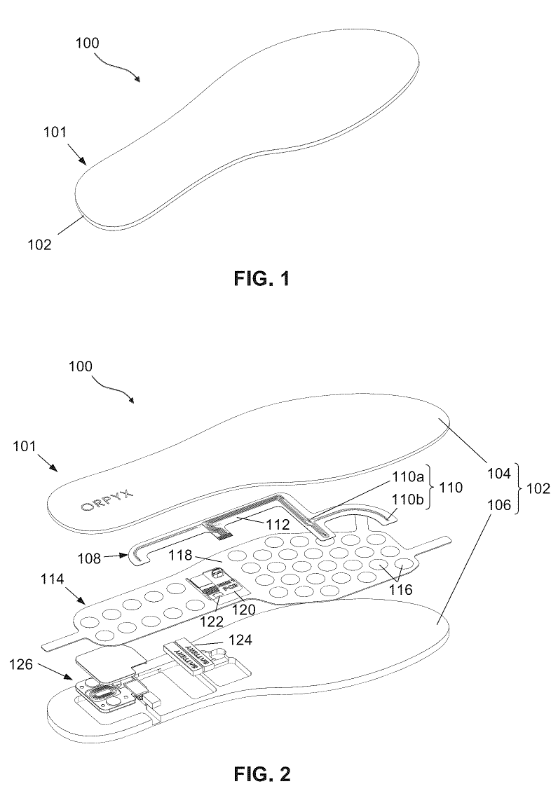

Referring to FIGS. 1 and 2, an example input device 100 in the form of an insole 101 is shown. The insole 101 may be worn in direct contact with the user's skin, or may be spaced from the user's skin (e.g. by a sock). Insoles usable in the processes and systems described herein may be of a variety of configurations, of which the insole 101 is but one example. For example, the insoles may be those available from Orpyx Medical Technologies Inc. (Calgary, Canada). For further example, the insoles may be those described in U.S. Pat. No. 10,004,428 (Everett et al.), International Patent Application Publication No. WO/2021/092676 A1 (Everett et al.), U.S. Pat. No. 11,700,904 (Stevens et al.), and/or United States Patent Application Publication No. 2023/0189918 (Stevens et al.). Each of the aforementioned documents is incorporated herein by reference in its entirety.

Referring first to FIG. 1, in the example shown, the input device 100, which can be insole 101, includes an insole bulk 102, which may be made up of one or more layers such as a cushion layer, a support layer, a gel layer, an anti-odor layer, a thermal insulation layer, and/or a foam layer. In the example shown, the input device 100 is in the form of a generic insole that can be used by a variety of users and in a variety of footwear. The insole bulk 102 includes a top layer 104 and a base layer 106 (shown in FIG. 2). The top layer 104 may in turn include multiple sub-layers, such as an upper finishing layer (not shown), a middle comfort layer (not shown), and a contoured layer (not shown). Likewise, the base layer may include multiple sub-layers (not shown).

In other examples, the insole 101 may be a different type of generic insole (e.g. a comfort insole, a flat insole, an athletic insole, a shock-absorbing insole, a sockliner, or a gel insole), the insole 101 may be an orthotic that is custom manufactured for a user, or the insole may be connected to any layer positionable under the user's foot. For an orthotic that is custom manufactured for a user, the user's foot may be assessed (e.g. by a podiatrist, optionally using plaster casting or 3D scanning), and the insole bulk 102 may be custom fashioned based on the assessment, for example in order to support the user's foot, improve foot function, relieve pain, and/or relieve pressure. Furthermore, the insole 101 may in some examples be integral with a shoe. The insole 101 may be available in different sizes.

The input device 100 may contain an array of sensors. In one embodiment, the sensors may be resistive sensors. Each resistive sensor in the array may have an electrical component having a resistive property that is sensitive to, and altered by, changes in a property of the environment external to the insole 101. The magnitude of the environmental property applied to each resistive sensor correlates to a measurable and predictable change in resistance of the resistive sensor. The array can include a single type of resistive sensor for detecting an environmental property, such as pressure sensors (e.g. force-sensing resistors, strain gauges), temperature sensors (e.g. thermistors), light-dependent sensors (e.g. photoresistors), flex sensors, or other types of resistive sensors.

Referring to FIG. 2, in the example shown, a temperature sensor array 108 is embedded in the insole bulk 102. The temperature sensor array 108 includes one or more temperature sensors 110 (i.e. a first temperature sensor 110a, a second temperature sensor 110b, and so on, only two of which are labelled) printed on flexible polymer film 112. The temperature sensor 110 or the temperature sensor array 108 may be printed directly onto the insole 101 or a portion of the insole 101. The temperature sensors 110 may be, for example, multilayer chip thermistors, and may have an accuracy of at least 0.2 degrees Celsius. The temperature sensors 110 may be configured to serially obtain measurements, for example continuously at a frequency (e.g. the frequency may be once per minute or any frequency that may help tune out fluctuations or noise in temperature readings over time collected by the input device 100), or intermittently at regular intervals, or sporadically. In one example, the frequency of data capture may be algorithmically informed by sensors included in the input system 100 (e.g. if from motion sensor measurements collected, the user is determined to be sitting, the sampling frequency may be at a higher rate, or if from the motion sensor measurements collected, the user is determined to be walking, the sampling frequency may be at a lower rate). In another example, the serial collection of temperature measurements from a wearable input device 100 will demonstrate temperature trends to a user or a third party which may otherwise go unnoticed.

As used herein, the term “temperature measurements” refers to the raw data generated by the temperature sensors 110. These temperature measurements may be processed downstream, for example by a processor as described below, to yield processed temperature measurements. “Temperature readings” may refer to temperature measurements or processed temperature measurements. The term “series of temperature readings” refers to temperature readings relating to measurements taken in series over a time period.

Referring still to FIG. 2 the input device 100 may include an optional pressure sensor array 114 embedded in the insole bulk 102. The pressure sensor array 114 may include one or more pressure sensors 116 (only two of which are labelled) printed on flexible polymer film 118. Each pressure sensors 116 may obtain continuous or frequent interval measurements and may produce a series of measurements. A high pressure sensor data rate (e.g. 64 Hz, 128 Hz or 256 Hz) may in some examples be preferred, to reduce pre-process noise occurring during data down sampling. In one example, a high data sensor data rate may enable quantification of user gait, activity, behavior, or posture, all of which may require a higher sampling rate. The pressure sensors 116 may be flat and flexible. For example, the pressure sensors 116 may be force sensing resistors, pressure sensors, piezoelectric tactile sensors, elasto-resistive sensors, capacitive sensors or more generally, any type of force sensor that can be integrated into an input device 100, such as insole 101. The number of pressure sensors 116 may depend on the size of the user's foot and may be determined by a set metric relating to the subject's foot. For example, the pressure sensors 116 may cover 25% of the surface area of the subject's foot. The pressure sensors 116 may also cover critical areas of the subject's foot. For example, the pressure sensors 116 may be denser in the metatarsal and heel areas.

In a further example, temperature sensors 110 and the optional pressure sensors 116 are disposed in the insole bulk so as to line up with the user's foot portion(s) remaining after amputation. For example, the resistive sensors are arranged in the insole bulk or a portion of insole bulk to align with a transmetatarsal amputation stump. In a specific example, unilateral temperature and pressure measurements are collected because the user has no right foot and the temperature sensors 110 and optional sensors 116 are located under the heel of the left transmetatarsal amputation stump.

As used herein, the term “pressure measurements” refers to the raw data generated by the pressure sensors 116. These pressure measurements may be processed downstream (e.g. normalized and/or standardized, such as normalized by the bodyweight of the subject), for example by a processor as described below, to yield processed pressure measurements “Pressure readings” may refer to pressure measurements or processed pressure measurements. The term “series of pressure readings” refers to pressure readings relating to measurements taken in series over a time period.

In the example shown, the input device 100 may further include at least a first optional IMU 122. In the example shown, the IMU 122 is integral with processor 120 (described below) and is shown schematically. The optional IMU 122 can include one or more sensors for measuring the position and/or motion of the user. For example, the IMU 122 may include one or more of a gyroscope, accelerometer (e.g., a three-axis accelerometer), magnetometer, orientation sensor (for measuring orientation and/or changes in orientation), angular velocity sensor, and inclination sensor. Generally, the IMU 122 includes at least an accelerometer. The optional IMU 122 may be included in any section of the insole bulk 102 or may be external to the insole bulk 102. For example, the IMU 122 may be included in the user's shoe or clipped to the user's shoelaces. More than one IMU 122 may be included within or external to the insole bulk 102 to provide more comprehensive information.

As used herein, the term “IMU measurements” is used herein to refer to the raw data generated by the IMU 122. These IMU measurements may be processed downstream, for example by a processor as described below, to yield processed IMU measurements. “IMU readings” may refer to IMU measurements or processed IMU measurements. The term “series of IMU readings” refers to IMU readings relating to measurements taken in series over a time period.

Referring still to FIG. 2, the input device 100 further includes a processor 120 that receives at least temperature measurements from the temperature sensors 110 (and optionally additional measurements from the pressure sensors 116, the IMU(s) 122, and other optional sensors). Readings from the temperature sensors 110 may include raw data such as resistance data, capacitance data, and/or other raw data. Likewise, measurements from the optional pressure sensors 116 and IMU 122 may include raw data. The processor 120 may apply an algorithm for reducing data noise, and/or for scaling/calibrating the measurements, to yield processed temperature measurements, or optional processed pressure measurements and IMU measurements. Communication between the processor and the various sensors may be through any wired or wireless connection.

In an alternative example, the temperature readings, and optional pressure readings and IMU readings may be transmitted to a remote processor (not shown) for processing (i.e. it is possible for no processing or for minimal processing to occur within the input device 100). The remote processor may be located on any suitable mobile or other device (e.g. smartphone, smartwatch, tablet, laptop computer, desktop computer, cloud-based server etc.). The remote processor may be used in addition to the processor 120 and may provide additional processing resources not available via the processor 120 or may be used instead of the processor 120.

One example of a remote processor is a cloud server, which may be used to provide additional processing capabilities. For example, some aspects of processing may be delegated to the cloud server to conserve power resources in the input device 100. The cloud server may store the temperature readings, and optional pressure readings and IMU readings and/or allow for more complex processing of the sensor data. In some cases, the cloud server, processor 120 and/or other remote processor may communicate in real-time to provide timely feedback to the user.

Additionally, the temperature readings and optional pressure readings and IMU readings may be transmitted through an optional external relay device (e.g. cellphone or bridging device), before being transmitted to the cloud server through a wired or wireless connection. For example, the external relay device may be a bridging device, which is plugged into a wall outlet and connected to the input device 100 via a wireless connection. The bridging device may receive data or readings from the input device 100 for transmission to the processor 120. The external relay device may store the temperature readings and optional pressure readings and IMU readings until transmission to a remote cloud server or output device is available. Additionally, the external relay device may be included in the remote processor device.

As will be described below, the processor 120 and/or remote processor may input at least the temperature readings and optional pressure readings and IMU readings into the foot health change detection algorithm (which may be stored in a non-transitory storage memory of the insole, or in a remote non-transitory storage memory).

Referring still to FIG. 2, a pair of batteries 124 are further embedded in the insole bulk 102, for providing energy to the components of the input device 100. The batteries 124 can be charged by a wireless charging assembly, which includes a wireless charging transmitter (not shown) and a wireless charging receiver 126 that is embedded in the insole bulk 102. The wireless charging receiver 126 is embedded in the heel of the input device 100 of FIG. 2, however other wireless charging receiver 126 locations may also be used. During power transfer, the wireless charging receiver 126 is placed in proximity to the wireless charging transmitter, which may be tuned to optimize the power transfer through the variable or invariable thickness of the insole bulk 102. If the wireless charging receiver 126 is used to power the input device 100, the batteries 124 are rechargeable batteries.

As noted above, various supplemental sensors may be included in the input device 100, for generating supplemental measurements. Such sensors may include one or more of a temperature sensor, a GPS sensor, a heart rate sensor, a respiratory rate sensor, a blood pressure sensor, a blood oxygen saturation sensor, a blood flow sensor, a blood or environmental content quantification sensor (e.g. glucose, electrolytes, minerals, oxygen, carbon dioxide, carbon monoxide, humidity, HbA1C, Ethanol, protein, lipid, carbohydrate, cortisol, lactate, pH, pro- and anti-inflammatory markers, MMPs, growth factors, bacterial content), a hydration status/tissue turgor sensor, a joint position sensor, a gait analysis sensor (including supination and pronation), a device breakdown sensor, a pedometer, an accelerometer, a velocity sensor, a calorimetry sensor, a centre of gravity sensor, a centre of foot position sensor, a friction sensor, a traction sensor, a contact area sensor, a connectivity/insulation sensor, an EEG sensor, a barometer, an altimeter, and/or an ECG sensor.

Optionally, the input device 100 may include one or more stimulators (not shown) for providing feedback to the user. The stimulator(s) may provide a tactile (e.g. vibratory), audible, or visual stimulus to the user.

The components of the input device 100 may be generally flat, and/or may be nested within pockets of the insole bulk 102, so that in use the user generally does not feel the presence of a foreign object under their foot.

In the example shown, the input device 100 is in the form of a single insole 101; however, in alternative examples, the input device 100 may be in the form of a pair of insoles (i.e. a left input device and a right input device, which can be worn concurrently).

In further alternative examples, the optional pressure sensors 116 and IMU 122 may be worn on the user's foot in another fashion other than via insole 101, such as a sock, shoe, or ankle bracelet, for example.

In further alternative examples, the temperature sensors 110 and the optional pressure sensors 116 and IMU 122 may not be worn on the subject's foot such as via the insole 101, but may be disposed in a non-wearable input device 100 with which the subject's foot comes into contact, such as a mat, a platform, a treadmill, or equipment.

As mentioned above, the input device 100 may be part of a system that includes an output device (not shown). The output device may be, for example, a display screen, a speaker, or a tactile stimulator (e.g. a vibratory device). The input device 100 may be in communication with the output device (optionally in direct communication, or in communication via the external relay device, cloud server, or remote processing device).

The output device may be configured to output an indication of the temperature readings and optional pressure readings and IMU readings, as described in further detail below.

Methods for detecting foot health changes using temperature readings will now be described. For clarity, the methods will be described with reference to input device 100; however, the methods are not limited to input device 100. In general, methods for detecting foot health changes may include: collecting a first temperature reading at a first plantar location on a user's body, at each time point over a time window, to yield a first series of temperature readings; collecting a second temperature reading at a second plantar location on a user's body, at each time point over a time window, to yield a second series of temperature readings; determining a temperature offset and applying the temperature offset to the first series of temperature readings and second series of temperature readings to yield a first series of offset temperature data and second series of offset temperature data; optionally identifying and removing irrelevant offset temperature data from the first series of offset temperature data and second series of offset temperature data to yield a first series of processed temperature data and second series of processed temperature data; determining a temperature difference at each time point in the time window by comparing the first series of processed temperature data and second series of processed temperature data; and generating a foot health change flag if the average of the temperature differences over the time period exceeds a foot health change threshold. The method may be stored in a non-transitory storage memory, which may be included in the input device 100 or may be remote from the input device 100. A processor (e.g. processor 120 or a remote processor as described above) receives the temperature readings from the temperature sensors 110, and inputs the temperature readings (either as raw measurements or processed measurements) into the processor 120 to be used in the foot health change detection method.

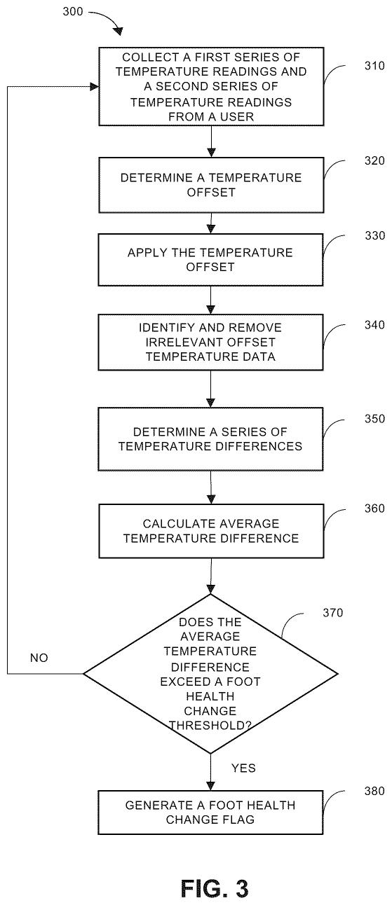

Referring now to FIG. 3, shown therein is a method for generating a foot health change flag when a foot health change for the is detected. The foot health change can include any disease, infection, disorder, or deformity of the foot, or improvement, reversal, or recidivism of any disease, infection, disorder or deformity of the foot or any other part of the body that can be detected by a wearable temperature sensor or a temperature sensor positionable on the foot (e.g. systemic circulatory or vascular blood flow issues such as a blocked or partially blocked artery in the leg may manifest as a temperature difference in the foot). The foot health change can also be used to evaluate the effectiveness of any intervention to address the foot health. The foot health change being detected may depend on the type of temperature difference and method used to determine the foot health change flag. For example, a contralateral temperature difference (e.g. left foot to right foot) may be used to generate a foot health change flag for inflammation, infection, perfusion differences (including narrowing, ischemia, hemorrhaging, acute or chronic changes in vascular status such as peripheral arterial disease, peripheral venous disease or peripheral perfusion), asymmetrical foot temperatures (including asymmetrical warmth or asymmetrical coolness), high pressures, or anatomical shapes (e.g. Charcot foot). An ipsilateral temperature difference (i.e. same foot) may be used to generate a foot health change flag for localized inflammation, localized coolness/ischemia, infection, perfusion differences (including narrowing, artery damage, ischemia, hemorrhaging, acute or chronic changes in vascular status), high pressure, friction, or an external cooling or heat source. In addition, a foot health change flag, a contralateral temperature difference, or an ipsilateral temperature difference may be used to evaluate changes in a user's foot temperatures in response to an intervention (e.g., revascularization procedure, medication, pharmacological therapy, activity/movement prescription, etc.) and to evaluate the intervention's effectiveness. In addition, a combination of a contralateral temperature difference and an ipsilateral temperature difference may be used to generate a foot health change flag localized inflammation, localized coolness/ischemia, infection, perfusion differences, high pressures, anatomical shapes, friction, or an external cooling or source.

The embodiment shown in method 300 is directed towards the detection of temperature changes caused by vascular or circulatory changes, inflammation, excessive pressure, or infection on the foot of the user. Specifically, the temperature sensors 110 may detect the symptoms of the foot health change, including detecting the skin temperature changes from inflammation, excessive pressure, or friction on various parts of the user's foot. A localized temperature increase or decrease may be a symptom of the foot health change and the area of the localized temperature increase or decrease may be detected using an array of temperature sensors 110 across the foot. For example, the foot health change may include diabetic foot ulcers (DFUs) resulting from a chronic disease complication such as peripheral neuropathy from diabetes mellitus. An increase in localized foot temperature may be a result of inflammation, which is a common symptom preceding DFUs. Another example of the foot health change may include inflammation caused by gangrene at the foot. Method 300 may be used to detect the foot health change by generating a foot health change flag indicating the presence of the symptom of the inflammation, excessive pressure, or infection. The method may be carried out with a wearable device, such as input device 100. Method 300 may be repeated to regularly check for or change the foot health change flag as the user's circumstances change (e.g. due to movement or worsening of condition, due to increases or decreases in user movement or activity, due to the onset of a new health complication or disease, due to the user using new shoes or a new assistive technology such as an offloading boot, or due to a new intervention such as medication, rehabilitation, surgery, procedure, etc.).

At step 310, a first temperature reading is collected from the user. The first temperature reading is collected at a first time point in a series of time points over a time period. The temperature readings are collected at each time point in the time period to yield a first series of temperature readings. The first series of temperature readings are collected at a first plantar location on the body of the user. Similarly, at each of the time points in the time period a second temperature reading is collected from a second plantar location on the body of the user to yield a second series of temperature readings. The temperature readings include at least the first series of temperature readings and the second series of temperature readings but may also include additional series of temperature readings. The first plantar location and the second plantar location may be on the same foot of the user or may be on opposite feet of the user (i.e. the first plantar location on a first foot and the second plantar location on a second foot). If the first plantar location and second plantar location are on opposite feet, both plantar locations may be under the same plantar structure. For example, the first plantar location may be under the first heel of the user and the second plantar location may be under the second heel of the user. Other plantar structures may include high risk areas or bony prominences of the foot where foot health changes are common because of high repetitive pressures. The high-risk areas may include the heel, metatarsal-phalangeal joints, toes (such as the hallux), and lateral side of the foot. For example, the input device 100 may have five temperature sensors 110 within the insole bulk 102 (as indicated in FIG. 2). The first plantar location and the second plantar location of the temperature sensors 110 may be located under one of the heel, first metatarsal, third metatarsal, fifth metatarsal, and hallux.

The time period may be any amount of time where a series of temperature readings are taken. The series of temperature readings may be consecutive, for example, temperature readings may be taken for the entire period when a user wears the input device 100. The series of temperature readings may also be non-consecutive, for example, temperature readings may be taken when a user dons and doffs the input device 100 throughout the time period, resulting in portions of temperature readings followed by sections of time with temperature readings at room temperature or with no temperature readings. In one example, the time period may be a data day, which is a 24-hour period (i.e. midnight from the previous day to midnight of the current day) during which temperature readings are recorded. In another example, the time period may be more than one data day. For example, the time period may be three data days to capture the user's temperature reading trends in foot health over a longer period. In a third example, the time period may be a portion of a data day. The time period may be a data session that starts when the user dons the input device 100 and ends when the user doffs the input device 100. The processor 120 may be used to clean the first series of temperature readings and/or the second series of temperature readings before any data manipulation is performed on the temperature readings. For example, a smoothing filter may be applied to the temperature readings to clean noise in the electrical circuits connected to the temperature sensors 110. In another example, interpolation methods may be applied to the temperature readings to fill in any missing or inaccurate temperature data. The missing or inaccurate temperature readings may be synthesized from the surrounding temperature readings (e.g. temperature readings collected within a range of consecutive time points).

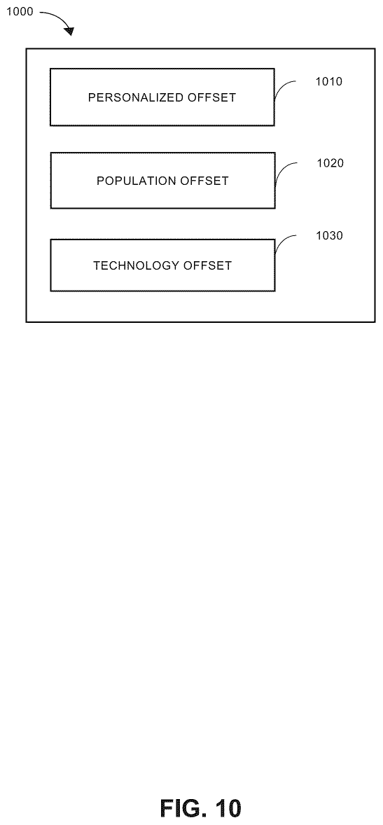

At step 320, a temperature offset is determined. The temperature offset is used to adjust the temperature readings from the temperature sensors 110 to increase the accuracy of the foot health change detection method. The temperature offset may be used to adjust temperature readings based on a variety of factors that may affect plantar temperatures, such as biological temperature differences, biases in the hardware, or drift in the hardware readings over time. The temperature offset may be determined using previous temperature readings and/or historical temperature readings from the input device 100. The method for determining the temperature offset may vary depending on the type of temperature offset being applied. More examples of temperature offsets and methods of determining the temperature offset are described in FIG. 10.

At step 330, the temperature offset is applied to the first series of temperature readings and the second series of temperature readings. The temperature offset is applied to the first series of temperature readings to generate a first series of offset temperature data. Additionally, the temperature offset is applied to the second series of temperature readings to generate a second series of offset temperature data. The temperature offset may be a single temperature offset, such as a single temperature offset applied to one or both series of temperature readings. The temperature offset may also include multiple temperature offsets applied together to one or both series of temperature readings. The temperature offset may further include a unique temperature offset applied to each series of temperature readings, separately, or the temperature offset may include the same temperature offset applied to each series of temperature readings. Step 330 may also be applied to any additional series of readings generated from the temperature sensors 110 in the input device 100. In another example, the temperature offset may be updated and change over time as additional temperature readings are collected.

At step 340, irrelevant offset temperature data is identified and removed from the first and second series of offset temperature data or by removing the corresponding temperature readings from the first and second series of temperature readings. The irrelevant offset temperature data includes the temperature readings causing inaccurate foot health change flags. Inaccurate foot health change flags may include false negative or false positive foot health change flags. A false negative foot health change flag is an incorrect indication of an absence of the foot health change when the foot health change is present. A false positive foot health change flag is an incorrect indication of when the foot health change is not present. For example, the irrelevant offset temperature data may include temperature readings collected when the insole is warming up to the temperature of the user's foot. The temperature readings collected when the insole is equilibrizing with the user's foot may not accurately represent the temperatures of the foot and may result in a false positive foot health change flag. In another example, the irrelevant offset temperature data may include data for which the insole is not reliably in contact with the foot. Including temperature reading when the insole is positioned on the foot may result in a false positive foot health change flag. Additional data preprocessing steps may be taken.

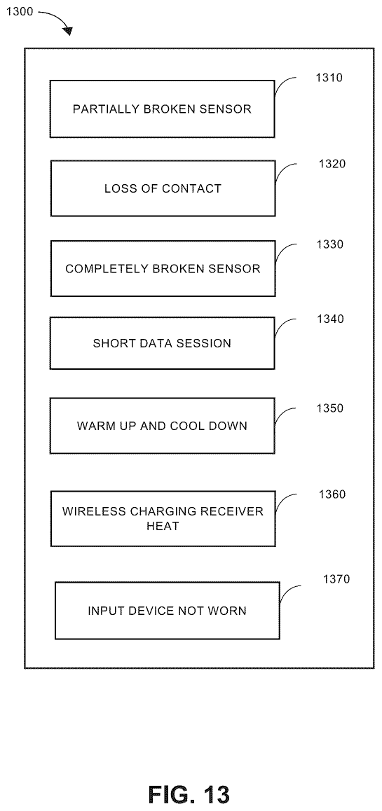

The irrelevant offset temperature data may be identified using one or more thresholds, which identify technology issues or inaccurate data events in the temperature readings. The irrelevant offset temperature data may include portions of offset temperature data that cannot be adjusted using the temperature offset. To address the non-adjustable portions of offset temperature data, the offset temperature data is removed from the data set and not used in step 350. Additional irrelevant offset temperature data examples are described in FIG. 13. A first series of processed temperature data is generated once the irrelevant offset temperature data is identified and removed from the first series of offset temperature data. Similarly, a second series of processed temperature data is generated once the irrelevant offset temperature data is identified and removed from the second series of offset temperature data. The irrelevant offset temperature data may be identified and removed from the first series of offset temperature data separately from the second series of offset temperature data. For example, the irrelevant offset temperature data for the first series of offset temperature data may be different than the irrelevant offset temperature data for the second series of offset temperature data. Alternatively, the same irrelevant offset temperature data may be identified in one series of offset temperature data and applied to each of the other series of offset temperature data. The irrelevant offset temperature data may be removed by replacing the offset temperature data point with a Not a Number (NaN) value in the processed temperature data. The NaN value may not be used to calculate the temperature difference in step 350, may return an illegal value (i.e. an error), or may return an NaN result.

At step 350, a temperature difference is determined. The temperature difference is determined by comparing the first series of processed temperature data and the second series of processed temperature data for each time point in the time period. However, the temperature difference may be determined by comparing one or more of the first series of processed temperature data or temperature readings to one more of the second series of processed temperature data or temperature readings. The temperature difference may be a contralateral temperature difference, an ipsilateral temperature difference, or some combination thereof. A contralateral temperature difference may be calculated between readings taken from a first plantar location on a first foot and a second plantar location on a second foot. The contralateral temperature differences may be applied to processed temperature data derived from temperature readings collected by temperature sensors under equivalent plantar structures of the opposite feet. For example, for a pair of insoles each with a five-sensor temperature sensor array 108, with a first temperature sensor array in the left insole and a second temperature sensor array in the right insole shaped in the mirror image of the first temperature sensor array, a contralateral temperature difference may be calculated by taking the difference between readings taken from a left foot temperature sensor and the corresponding mirror image right foot temperature sensor. The contralateral temperature difference may be similarly calculated for the readings taken from the remaining temperature sensors of the first temperature sensor array and the corresponding mirror image temperature sensors of the second temperature sensor array.

An ipsilateral temperature difference may be calculated between a first temperature sensor 110a and second temperature sensor 110b on the same foot of the user. When determining the ipsilateral temperature difference calculation, the second series of temperature readings may include calculating the average of some or all of the first and second series of processed temperature data, offset temperature data, or temperature readings (e.g. one temperature reading from the first series of temperature readings is compared to the average of five temperature readings from the second series of temperature readings). Alternatively, the ipsilateral temperature difference may include calculating the difference between the first series of processed temperature data and the average of the first and second series of processed temperature data or temperature readings. The first series of processed temperature data or temperature readings may be collected by a first temperature sensor 110a and a second temperature sensor 110b. In one example, the first series of processed temperature data or temperature readings may be collected by a temperature sensor at first plantar location related to a first angiosome of the foot and the second series of processed temperature data or temperature readings may be collected by a temperature sensor at a second ipsilateral plantar location related to a second angiosome of the foot. In other examples, the input device 100 includes a temperature sensor array 108 with more than two temperature sensors. Temperature readings may be taken at a plurality of plantar locations on the user's first foot and the second temperature reading at the second plantar location may include a series of average temperature readings, which is determined by calculating an average of the plurality of temperature readings collected by the input device 100 at each time point over the time period. For example, for an insole on the first foot of the user with a five-sensor temperature sensor array 108, a series of average temperature readings may be calculated from the five temperature sensors 110 for each time point in the time period. The series of average temperature readings may be used to calculate an ipsilateral temperature difference for the processed temperature data from each of the five temperature sensors 110 at each time point in the time period. In another example, temperature differences may be determined between a first series of average temperature readings taken from temperature sensors at a first plantar location related to a first angiosome of the foot and a second series of average temperature reading taken from temperature sensors at a second ipsilateral plantar location related to a second angiosome of the foot, each series of average temperature readings being determined by calculating an average of the plurality of temperature readings collected by the input device 100 at each time point over the time period.

Various other temperature differences may be determined at step 350. In a specific example, the temperature difference may be determined by calculating a difference between the processed temperature data from each of the temperature sensors 110 and ambient temperature readings at each time point. In another example, the temperature difference may be determined by calculating a difference between the average temperature of the temperature sensors 110 of the left input device and the average temperature of the temperature sensors 110 of the right input device. Other examples may include combinations of temperature differences.

At step 360, an average temperature difference is calculated. The average temperature difference is calculated from the average of the temperature differences at each time point in the time period. The average temperature difference calculation may be determined by generating a weighted average, non-weighted average, Or another descriptive summary analytic (e.g., median) of the temperature differences. Any temperature difference resulting in an NaN value, missing value, or error in step 370 may be ignored when calculating the average temperature difference. In other examples, the NaN value, missing value, or error may be replaced with a synthesized or estimated real temperature difference value using interpolation or some other method.

At step 370, the average temperature difference is compared to a foot health change threshold. The average temperature difference or temperature differences may be converted to absolute values, or the temperature differences may be reversed to produce positive values. For example, the temperature differences may be reversed by switching the calculation from left temperature readings subtracted from right temperature readings to right temperature readings subtracted from left temperature readings, or vice versa. Alternatively, the absolute value of the temperature differences may convert all the temperature readings to positive values for determining the foot health change flag. The foot health change threshold may be a single value (e.g. 2.2 degrees Celsius) and the temperature differences may be compared to the single threshold value. For example, the ipsilateral temperature difference of the first temperature sensor 110a may be −4 and converted to an absolute value of 4, which may be compared to the single positive threshold value. The example in FIG. 3 shows a method for a single threshold value, which may be exceeded. If the average temperature difference exceeds the foot health change threshold, the method proceeds to step 380. If the average temperature difference does not exceed the foot health change threshold, method 300 does not proceed any further, and is instead repeated from step 310. The user's foot may not have the indication or symptom of the foot health change when the average temperature difference does not exceed the foot health change threshold. The foot health change threshold may be related to an accepted value indicating early warning signs of a foot health change. For example, the foot health change threshold for a DFU may be at least 2 degrees Celsius because a temperature difference of at least 2 degrees Celsius between a first plantar location and a second plantar location may be an early indicator of a DFU foot health change. In another example, the foot health change threshold may be related to the improvement, reversal, or recidivism of a foot health change such as revascularization after medication administration, a vascular procedure, or other intervention. If a temperature difference falls below the foot health change threshold, the success of the foot health change procedure administered may be confirmed.

The example method 300 and the following methods 400, 500, and 700 describe a single positive threshold value. However, the foot health change threshold may also include a set of two threshold values. Particularly, the foot health change threshold may include a mirrored set of negative and positive threshold values. The temperature difference calculation may result in negative values and positive values. The negative values may be compared to a negative threshold value (e.g. −2.2 degrees Celsius) and the positive values may be compared to a positive threshold value (e.g. 2.2 degrees Celsius). The method may proceed to step 380 when the average temperature difference falls below the negative threshold value or exceeds the positive threshold.

Instead of the average temperature difference, the method may use a percentage of the temperature differences compared to the foot health change threshold. For example, the temperature difference at each time point may be compared to the foot health change threshold. If over a certain percent (e.g. greater than 50%) of the temperature differences exceed the foot health change threshold, the method may proceed to step 380. If the percent of temperature differences exceeding the foot health change threshold does not exceed the certain percent, the method repeats from step 310. In the example, the average temperature difference may not be calculated, and step 370 may be replaced by summing the number of individual temperature differences that exceed the foot health change threshold. The sum may be used to determine the percentage of temperature differences exceeding the foot health change threshold.

The foot health change threshold may be different for each type of temperature difference. For example, an ipsilateral temperature difference may be compared to a foot health change threshold of 2.0 degrees Celsius and a contralateral temperature difference may be compared to a foot health change threshold of 2.2 degrees Celsius. An ipsilateral temperature difference may be compared to a foot complication threshold of 1.0 degrees Celsius for differences take between two different angiosomal areas of the foot. The foot health change threshold may also be personalized to the user's temperature reading baseline. The baseline temperature readings may be collected over a previous time period and used to determine a personalized foot health change threshold. A user with a biological temperature difference of 1.0 degrees Celsius between the left and right foot may have a foot health change threshold of 3.2 degrees Celsius for contralateral temperature differences (i.e. personalized baseline, 1.0 degrees Celsius, added to the accepted threshold value, 2.2 degrees Celsius). Additionally, the foot health change threshold may change over time. For example, the foot health change threshold may be increased or decreased to improve accuracy as more temperature readings are collected. As a further example, a personalized foot health change threshold may be adjusted or updated over time.

In a specific example, the average temperature difference or series of temperature differences may be determined for a moving window. The moving window may be a 15-minute time window, which ends at the current instant. The moving window may change as additional temperature readings are collected and the current instant changes. The average temperature difference may be calculated over the moving window and continuously updated each time the current instant changes and the time window moves. The average temperature difference at each instant may be compared to the foot health change threshold. In another example, the foot health change threshold may be determined from the average temperature difference over the moving time window. That is, the foot health change threshold may be continuously updated at each instant. The deviation from the average temperature difference may be determined and compared to a deviation threshold. The deviation may capture any sudden spikes in temperature from the foot health change. Similarly, the foot health change threshold may be determined using the same type of temperature differences or other types of temperature differences from the same time period. The average temperature difference over the moving time window may be calculated in addition to the average temperature difference over the time period.

At step 380, a foot health change flag is generated. The foot health change flag is generated to indicate the emergence or pre-emergence of the foot health change (or improvement, reversal, or recidivism of the foot complication) to the user and/or a third party. The third party may include a remote patient monitor or a third party. The foot health change flag may be presented visually on an output device of the system, such as a display, or it may be provided audibly or haptically. The display may be presented as a dashboard where a user or multiple users are shown the various foot health change flags. The third party may view the dashboard to determine if one or more of the users require treatment to avoid “unsafe conditions”. In some examples, the foot health change flag may also include an alert to notify the user. A foot health change flag may include an indication of the location of the plantar location of the foot health change. For example, the display may include a general foot-shaped image and the location of the foot health change may be shown with a visual indication (e.g. bright color) at the location matching the plantar location of the foot health change flag. More than one foot health change flag may be displayed if the foot health change flags are generated more than once or if there is a foot health change flag at more than one plantar location. The display may also indicate the type of temperature difference calculation used to generate the flag. For example, the display may indicate the type of temperature difference calculation that resulted in the foot health change flag. The display may also include various additional information derived from the temperature readings or optional pressure readings or IMU readings. For example, the average temperature for all the temperature sensors 110 in the left or right input device 100 may also be reported to the user and/or third party. The average value for each foot may be recorded and/or tracked in an average temperature report. Additionally, the average values of each of the temperature sensor over the data session or time period may also be recorded. Other temperature reading information may include various temperature differences, such as the temperature difference for the first half of wear and second half or wear, or the temperature difference for different times of the day, or the temperature differences at different durations of wear.

The third party may initiate a care action after the foot health change flag is generated. The care action may be based on the type of foot health change flag. For example, the care action may include a suggestion for gait modification (e.g. balancing out weight across the user's two feet), which may decrease the friction on a specific part of the foot causing inflammation. Another care action may include an adjustment to the input device 100. For an insole 101, the adjustment may include an orthotic adjustment to redistribute the user's foot pressure or check on the tightness of the user's shoelaces. In other examples, the third party may initiate a care action for the user to book an appointment with the third party.

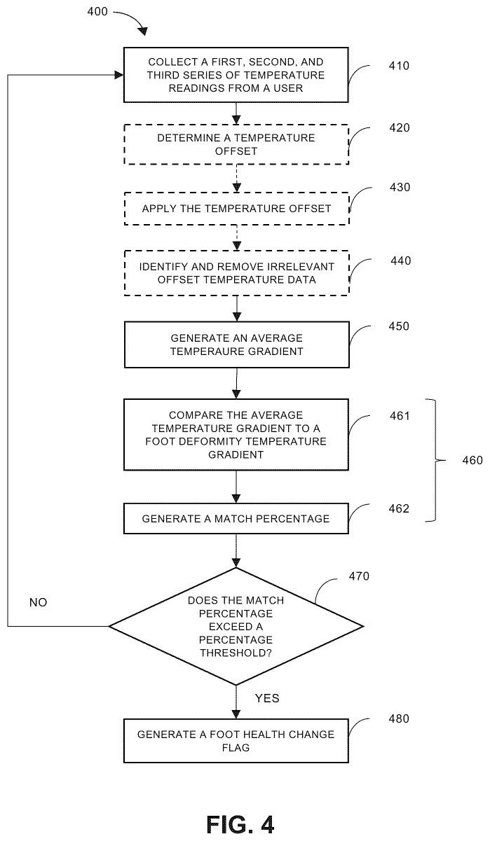

Referring now to FIG. 4, shown therein is example method 400, which applies a similar method to Method 300 of FIG. 3. Method 400 is directed towards generating a foot health change flag when a foot health change caused by a foot deformity is detected. The foot deformity may cause increased friction at the affected area of the foot, which may result in increased localized heat at or near the location of the foot deformity. The increase in localized heat may be detected using method 400 by comparing the temperature gradient of the user to a temperature gradient indicating the foot deformity. The foot deformity foot health change may include, for example, Charcot foot, hammer toe, claw toe, mallet toe, flatfoot, high arches, heel spur, splayfoot, pronated foot, equinus foot, partial foot amputation, and bunion, or a combination of these deformity foot health changes. The foot biomechanics foot health change may be detected from the temperature changes caused by the variable friction at a portion of the user's foot. The foot biomechanics foot health change may be detected during user motion (e.g. walking), which enhances the temperature resulting from friction. Example method 400 may be applied by input device 100. Method 400 may be repeated to regularly update the foot health change flag.

At step 410, temperature readings are collected from the user. The temperature readings include a first series of temperature readings, a second series of temperature readings, and a third series of temperature readings. The temperature readings may be collected from the temperature sensors 110 of input device 100. At each of a series of time points over the time period, a first temperature reading is collected at a first plantar location on the body of the user, to yield a first series of temperature readings. Additionally, at each of a series of time points over the time period, a second temperature reading is collected at a second plantar location on the body of the user, to yield a second series of temperature readings. Similarly, at each of a series of time points over the time period, a third temperature reading is collected at a third plantar location on the body of the user, to yield a third series of temperature readings. The first plantar location, the second plantar location, and the third plantar location may all be on the same foot of the user (i.e. a first foot of the user). The first, second, and third plantar locations may include high risk areas or bony prominences of the foot where foot health changes are common because of high repetitive pressures. For example, the first plantar location, the second plantar location, and the third plantar location may be each located under one of the heel, first metatarsal, third metatarsal, fifth metatarsal, and hallux of the user.

At optional step 420, a temperature offset is determined. The temperature offset is used to adjust the temperature readings to increase accuracy of the foot deformity foot health change. The temperature offset may be determined using historical wear session temperature readings from the user or historical temperature readings from the input device 100. The method for determining the temperature offset may vary depending on the type of temperature offset being applied. Examples of temperature offsets and methods of determining temperature offsets are described in FIG. 10.

At optional step 430, the temperature offset is applied to the first, second, and third series of temperature readings. The temperature offset may be applied to the first series of temperature readings to generate a first series of offset temperature data. Additionally, the temperature offset may be applied to the second series of temperature readings to generate a second series of offset temperature data and applied to the third series of temperature readings to generate a third series of offset temperature data. The temperature offset may be a single temperature offset, such as a single temperature offset applied to one or more series of temperature readings. The temperature offset may also include multiple temperature offsets applied together to one or more series of temperature readings. The temperature offset may further include a unique temperature offset applied to each series of temperature readings, separately, or the temperature offset may include the same temperature offset applied to each series of temperature readings.

At optional step 440, irrelevant offset temperature data is identified and removed from the first and second series of offset temperature data or by removing the corresponding temperature readings from the first and second series of temperature readings. The irrelevant offset temperature data may be identified using one or more thresholds. Examples of the filters that may be used to identify irrelevant offset temperature data are described in FIG. 13. A first series of processed temperature data is generated once the irrelevant offset temperature data is removed from the first series of offset temperature data. Similarly, a second series of processed temperature data is generated once the irrelevant offset temperature data is removed from the second series of offset temperature data and a third series of processed temperature data is generated once the irrelevant offset temperature data is removed from the third series of offset temperature data. The irrelevant offset temperature data may be identified and removed from the first series of offset temperature data, the second series of offset temperature data, and the third series of offset temperature data, individually. Alternatively, the same irrelevant offset temperature data may be identified in one series of offset temperature data and applied to each of the other series of offset temperature data. The irrelevant offset temperature data may be removed by replacing the offset temperature data points identified as irrelevant offset temperature data with an NaN value, missing value and ignored or may be replaced with a synthesized or estimated real temperature difference value using interpolation or some other method.

Additional data preprocessing steps may be taken. At step 450, an average temperature gradient is generated. The average temperature gradient is generated by ordering a set of temperature readings or optional processed temperature data (in the case steps 420 to 440 are included). The gradient may be a spatial intra-foot temperature gradient over the entirety or a portion of the user's foot. The average calculation is completed by summing all the temperature readings or optional processed temperature data for one member or one temperature sensor and dividing the result by the number of time points. The NaN values may not be counted as valid temperature readings or processed temperature data points. The average temperature gradient includes an ordered set of an average of the first series of temperature readings or processed temperature data, an average of the second series of temperature readings or processed temperature data, and the average of a third series of temperature readings or processed temperature data. The resulting average temperature gradient is an ordered set of at least three members.

At step 461, the average temperature gradient is compared to a foot deformity temperature gradient. Each member of the average temperature gradient is compared to a respective member in the foot deformity temperature gradient. The foot deformity temperature gradient may also be an intra-foot spatial temperature gradient for a foot. A member of the average temperature gradient may be a section or area of the user's foot in proximity to at least one temperature sensor. Each member of the average temperature gradient may be linked to one or more of the temperature sensors 110 in the input device 100 on a user's foot. For example, there may be five members in the set of the average temperature gradient for an input device with five temperature sensors 110. Each member of the ordered set of the average temperature gradient corresponds to a respective member in the foot deformity temperature gradient. The respective member in the foot deformity temperature gradient may be located at the same plantar location as the member in the average temperature gradient. Step 461 is the first part of the generation of a comparison metric 460 for determining if the symptom of the foot deformity foot health change is present.

At step 462, a match percentage is generated. The match percentage represents the level of equivalence between the average temperature gradient and the foot deformity temperature gradient. The memory or storage of the input device 100 may include one or more foot deformity temperature gradient profiles to compare to the user's average temperature gradient. The comparison may be used to determine the type of foot deformity. For example, the memory may include the foot deformity gradient for charcot foot, hammer toe, and flatfoot. Method 400 may be used to determine if flatfoot is present on the user's foot and alert a third party to the presence of the flatfoot. The match percentage may be generated for each member of the ordered set of the average temperature gradient by comparing each member of the ordered set of the average temperature gradient to each respective member of the foot deformity temperature gradient. The foot deformity temperature gradient may be determined by collecting temperature readings from users with a confirmed presence of the chosen foot deformity. A third party may confirm the presence of the foot deformity. In other examples, the temperature readings may be collected from multiple users with the confirmed presence of the chosen foot deformity and an average temperature gradient from the users' temperature readings may be used to determine the foot deformity temperature gradient. In an additional example, a known symptom of a foot deformity may cause an increase in skin temperature at a certain location of the foot. A synthesized foot deformity gradient may be developed using the temperature differences between a healthy foot and a foot with the foot deformity. Step 462 is the second part of the generation of a comparison metric 460 for determining if the symptom of the foot deformity foot health change is present.

The match percentage may be generated using various methods, depending on the type of foot deformity being detected. Some foot deformities may occur in multiple areas of the foot. For example, a hammertoe foot deformity may occur on the second, third or fourth toes of the user. The symptoms of hammertoe may be used to develop multiple foot deformity temperature gradients for the hammertoe foot deformity. The user's average temperature gradient may be compared to the foot deformity temperature gradient to detect the foot deformity in each of the common foot locations.

At step 470, the match percentage is compared to a percentage threshold. The average temperature gradient of the user may not exactly match the foot deformity temperature gradient, even when the user does have the foot deformity foot health change. An exact temperature gradient match may not be required for a foot health change flag to be generated for the user. Instead, the match percentage may be compared to a percentage threshold to allow for natural mechanical, anatomical, and physiological differences. The natural foot differences between one user's foot and another user's foot may cause a different presentation of the foot deformity (i.e. the degree of temperature difference). The percentage threshold may be used as a tolerance between the average temperature gradient and the foot deformity gradient, which may assist with preventing false negatives (i.e. a foot health change flag is not generated when the foot deformity is present). The percentage threshold may be altered for different foot deformities and may be based on the likelihood of symptom consistency from user to user. For example, the foot deformity temperature profile for hammertoe may be consistent across multiple users with various anatomical and physiological foot differences. In the example, the percentage threshold may be a high threshold, such as 90%. If the match percentage does not exceed the percentage threshold, the method does not proceed any further and instead repeats from step 410. If the match percentage exceeds the percentage threshold, the method proceeds to step 480.

At step 480, a foot health change flag is generated. The foot health change flag for the foot deformity foot health change may include an alert. The foot health change flag may be presented to the user and/or to a third party. Some of the foot deformity foot health changes may occur in various areas of the foot and the foot health change flag may be generated where the foot deformity is identified. The location of the foot deformity on the user's foot may be identified as a foot health change flag on a digital model of a foot, lower limb or other body part. The digital model of the foot may be reviewed by the user and/or a third party. The average temperature gradient and the location of the foot health change flag may be indicated on a thermal image display of the foot, lower limb or other body part on the dashboard. For example, a hammertoe foot deformity may occur on the second, third, or fourth toe. An input device 100 with temperature sensors for each toe may be indicate which toe has the hammertoe foot deformity. The location of the foot deformity may be identified using the members in the average temperature profile. A member with a high match percentage to the respective member in the foot deformity profile may be used as the location indicator for the foot health change flag. In other examples, the foot health change flag for the foot deformity may be presented as a general alert to the user, without an indication of the location of the foot deformity.

In addition to generating the foot health change flag, a personalized temperature offset may be calculated using the match percentage of each member of the ordered set of the average temperature gradient. The personalized temperature offset may be used to offset the temperature readings for the members with a match percentage exceeding the percentage threshold. The foot deformity in the members exceeding the percentage threshold may be elevated and prevent the input device 100 from accurately detecting inflammation (as described in method 300). The foot health change flag for the foot deformity may be indicated to a third party, and the third party may initiate a personalized temperature offset to prevent any further alerting. Alternatively, the personalized temperature offset may be applied as part of step 330 in method 300. A personalized temperature filter may also be calculated to remove irrelevant temperature readings caused by the foot deformity. Both the personalized temperature offset and the personalized temperature filter may be used to prevent excessive foot health change flag generation for a user with a confirmed foot deformity. The input device 100 may then be used to detect worsening of the foot health change and/or additional foot health changes.

In alternative examples, a temperature difference between a member in the average temperature gradient and the foot deformity temperature gradient may be used to determine if a foot deformity foot health change is present. The temperature difference method for a foot deformity may be similar to method 300 in FIG. 3. For example, a flatfoot foot deformity temperature gradient may have threshold values on the midfoot because the symptoms of flatfoot are present in the midfoot. The temperature difference may be determined by comparing the average temperature difference between a user's foot and a foot deformity temperature gradient for members located at the midfoot. The average temperature difference may be compared to a threshold for the members located at the midfoot of the average temperature difference. If the threshold is exceeded, the foot health change flag at step 480 may be generated. In some examples, the foot health change threshold may be different for various areas of the foot. For example, the heel member temperature difference may have a higher foot health change than the midfoot member temperature difference.

In some examples, the data from method 400 may be used to create a new input device 100 or adjust a pre-existing input device 100 for the user. In a first case, a new pair of insoles may be manufactured for the user when the foot health change flag for a specific foot deformity is generated. The new pair of insoles may be customized to the user's specific foot deformity to improve the user's foot biomechanics and prevent any further foot health changes. The custom insoles may be manufactured when the user's percentage match in one of the members exceeds the percentage threshold. For example, the user may have a foot health change flag generated in the midfoot member. The midfoot foot health change flag may indicate a flatfoot foot deformity and trigger an alert to a remote patient monitor to initiate the manufacture of custom insoles with arch support. The remote patient monitor may initiate the manufacture of the custom insoles based on a three-dimensional profile of the user's foot. In a second case, a new or pre-existing pair of insoles may be manufactured using the match percentages of each member. The percentage match of each member in the ordered set of the average temperature gradient may be used to calculate a custom insole adjustment factor. A pre-existing pair of insoles or a non-customized new pair of insoles may be adjusted based on the custom insole adjustment factor. For example, the user may have a pre-existing pair of custom insoles shaped to the user's feet. The foot health change flag for a specific foot deformity may indicate the insoles should be changed to adapt to a newly developed foot deformity. The foot health change flag may initiate a change to the pre-existing custom insoles. For example, a three-dimensional model of a generic flat insole may be adjusted based on the custom insole adjustment factor. The updated custom insole may be worn by the user to equalize the plantar pressures at the foot deformity, which may prevent worsening of the foot health changes or prevent the development of new foot health changes.

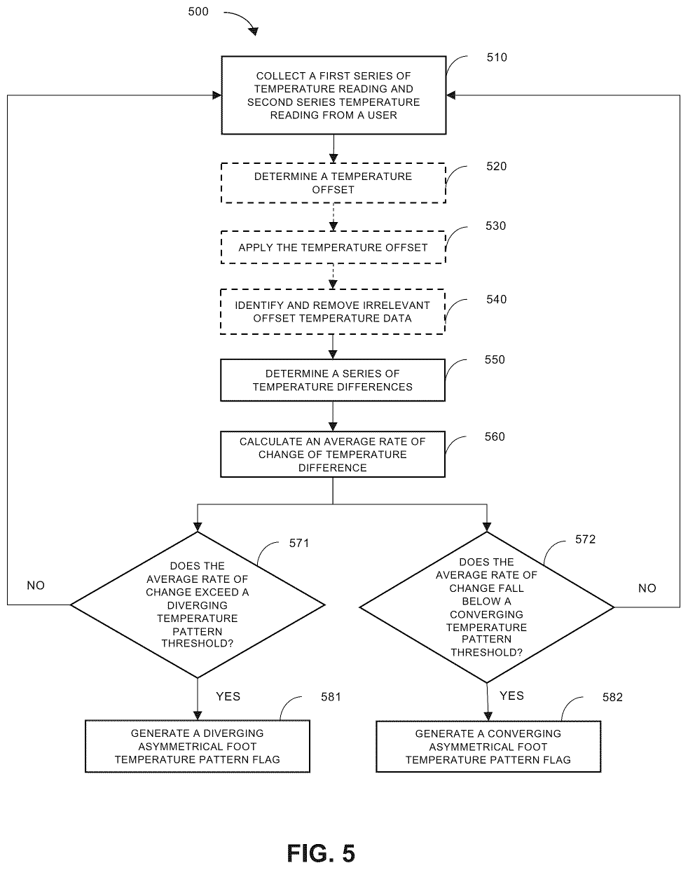

Referring now to FIG. 5, shown therein is example method 500, which also applies a similar method to method 300 of FIG. 3 and is directed towards temperature generating an asymmetrical foot temperature pattern flag when an asymmetrical foot temperature pattern is detected. The asymmetrical foot temperature pattern may indicate an asymmetrical vascular disease foot health change. The asymmetrical foot temperature pattern foot health change is a temperature difference between two plantar locations of the user caused by, for example, microvascular disease, macrovascular disease, or both in the feet or lower limbs of the user. The two plantar locations of the user may include, a first location on the left foot and a second location on the right foot of the user. In one example, the first location on the left foot is contralateral to the second location on the right foot of the user's feet. Alternatively, the two plantar locations of the user may include a first plantar location relating to a first angiosome of one foot of the user and a second ipsilateral plantar location relating to a second angiosome of the foot of the user. The asymmetrical vascular disease foot health change may present as a converging or diverging temperature pattern. A diverging temperature pattern occurs when the contralateral temperature difference between the two plantar locations of the user starts at a value and increases over the time period. A converging temperature pattern occurs when the contralateral temperature difference between two plantar locations of the user starts at a value and decreases over time. That is, there may be two different foot health change flags: a first converging asymmetrical temperature pattern flag and a second diverging asymmetrical temperature pattern flag. Diverging and converging temperature patterns may cause inaccurate foot health change flags in method 300 of FIG. 3. Example method 500 may be applied by input device 100. Method 500 may be repeated to regularly update the foot health change flag.

At step 510, temperature readings are collected from the user. The temperature readings may include a first series of temperature readings and a second series of temperature readings. The temperature readings may be collected from the temperature sensors 110 of input device 100. At each of a series of time points over the time period, a first temperature reading at a first plantar location on the body of the user is collected to yield a first series of temperature readings. The first plantar location may be located on a first foot of the user. Similarly, at each of a series of time points over the time period, a second temperature reading at a second plantar location on the body of the user may be collected to yield a second series of temperature readings. The second plantar location may be located on the first or a second foot of the user. The first and second plantar locations may include high risk areas or bony prominences of the foot where foot health changes are common because of high repetitive pressures. A time period for temperature collection from the user can have any length and the first and second series of temperature readings from a user may be collected over one or multiple time periods. For intra-day analysis of temperature differences, the collection of temperature readings from the user may occur over one or multiple time periods such as over a span of 1-24 hours.

At optional step 520, a temperature offset is determined. The temperature offset may be used to adjust the temperature readings from the temperature sensors 110 to increase the accuracy of foot health change detection. The temperature offset may be determined using previous temperature readings from the user or historical temperature readings from the input device 100. The method for determining the temperature offset may vary depending on the type of temperature offset being applied. Examples of temperature offsets and methods of determining temperature offsets are described in FIG. 10.

At optional step 530, the temperature offset is applied to the first series of temperature readings and the second series of temperature readings. The temperature offset may be applied to the first series of temperature readings to generate a first series of offset temperature data. The temperature offset may also be applied to the second series of temperature readings to generate a second series of offset temperature data. The temperature offset may be a single temperature offset, such as a single temperature offset applied to one or more series of temperature readings. The temperature offset may also include multiple temperature offsets applied together to one or more series of temperature readings. The temperature offset may further include a unique temperature offset applied to each series of temperature readings, separately, or the temperature offset may include the same temperature offset applied to each series of temperature readings.

At optional step 540, irrelevant offset temperature data is identified and removed from the first and second series of offset temperature data or by removing the corresponding temperature readings from the first and second series of temperature readings. The irrelevant offset temperature data may be identified using one or more thresholds, as described in step 340 of method 300. Examples of irrelevant offset temperature data are described in FIG. 13. A first series of processed temperature data is generated once the irrelevant offset temperature data is removed from the first series of offset temperature data. Similarly, a second series of processed temperature data is generated once the irrelevant offset temperature data is removed from the second series of offset temperature data. The irrelevant offset temperature data may be identified and removed from the first series of offset temperature data and the second series of offset temperature data, individually. Alternatively, the irrelevant offset temperature data may be identified in one of the series of temperature readings and applied to both series of temperature readings. The irrelevant offset temperature data may be removed by replacing the offset temperature data points identified as irrelevant offset temperature data with a NaN value or missing value and ignored or may be replaced with a synthesized or estimated real temperature difference value using interpolation or some other method. Additional data preprocessing steps may be taken.

At step 550, a temperature difference is determined, similar to step 350 of method 300. The temperature difference is calculated by comparing the first series of temperature readings or the optional first series of processed temperature data (i.e. in the case steps 520 to 540 are used) and the second series of temperature readings or the optional second series of processed temperature data (i.e. in the case steps 520 to 540 are used). The temperature difference may be calculated for each time point in the time period to generate a series of temperature differences.

At step 560, the average rate of change of the temperature difference is calculated over the time period. The average rate of change may be determined by calculating the rate of change between every two consecutive temperature readings and then taking the average of the rate of change calculations. The consecutive temperature readings may be consecutive time points. The rate of change may be calculated by taking the linear difference between the two temperature readings and dividing the resulting value by the difference between the time points of the two temperature readings. In another example, the rate of change may be determined by taking the average of the rate of change between every set number of consecutive temperature readings (e.g. every 10 temperature readings). A weighted average may be used, where temperature readings are weighted by usage duration, step count, or some other measure of patient behavior. In another example, the rate of change may be determined by finding a modelled linear or non-linear fit of a set of historical data. The rate of change may be calculated by removing outliers or irrelevant data points, cleaning the data, or transforming the data prior to the rate of change calculation. A correlation or other measure of association may be computed to quantify the strength of the rate of change calculation. Step 560 may proceed to both step 571 or step 572, which are used to determine a diverging or converging temperature pattern, respectively.

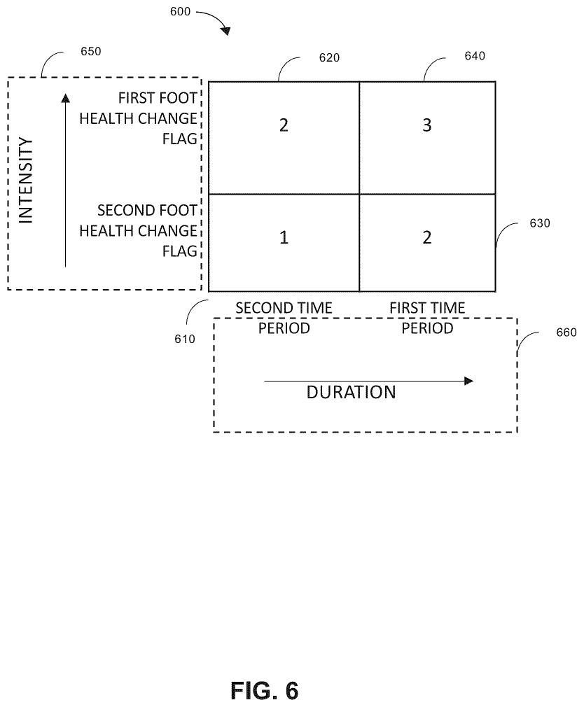

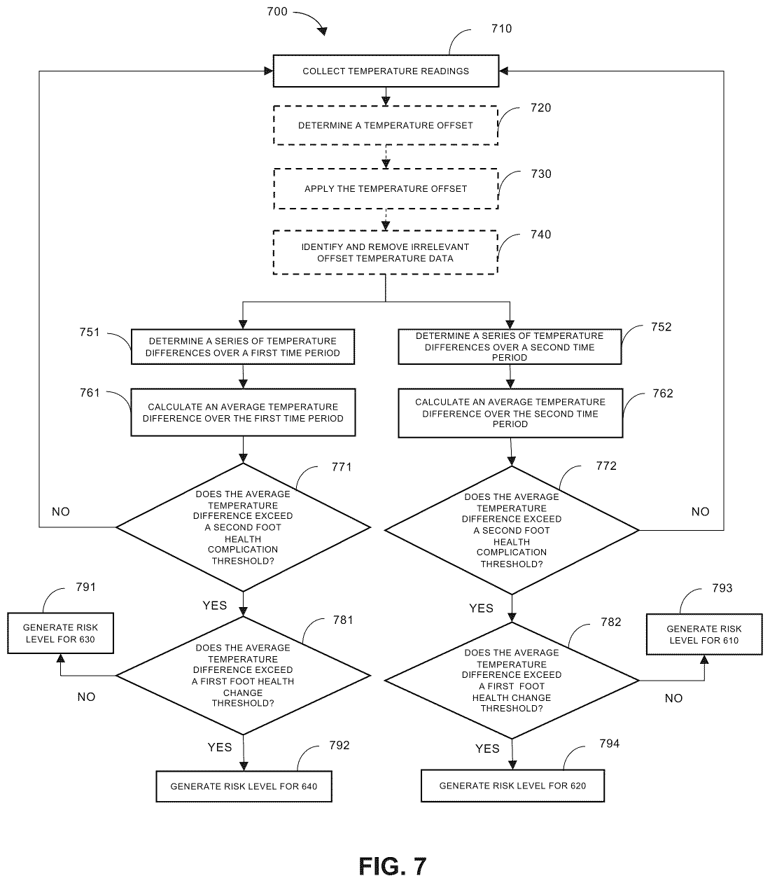

At step 571, the average rate of change of the series of temperature differences over the time period is compared to a diverging temperature pattern threshold. The diverging temperature pattern threshold may be used to identify an increase in the trend of the temperature differences over time. If the average rate of change of the series of temperature differences over the time period does not exceed the diverging temperature pattern threshold, the method does not proceed any further and instead returns to step 510 for method 500 to repeat from the beginning. If the average rate of change of the series of temperature differences over the time period exceeds the diverging temperature pattern threshold, the method proceeds to step 581. In an example, the average rate of change may also be compared to the diverging temperature pattern threshold at portions of the time period. For example, the rate of change during the first 50% of the wear session may be compared to the rate of change for the last 50% of the wear session. The method may proceed to step 581 when the difference between the rates of change exceeds the diverging temperature pattern threshold. The difference between the rates of change at the different portions of the time period may be compared to a diverging temperature pattern threshold. There may also be more than one diverging temperature pattern threshold to determine the severity of the asymmetrical temperature pattern. For example, there may be a higher and lower diverging temperature pattern threshold for detecting the risk level of the asymmetry, similar to Table 600 in FIG. 6 and the method for determining the risk level may be similar to method 700 in FIG. 7.

At step 572, the average rate of change of the series of temperature difference over the time period is compared to a converging temperature pattern threshold. The converging temperature pattern threshold may be used to identify a decrease in the trend of the temperature differences over time. If the average rate of change of the series of temperature differences over the time period does not fall below the converging temperature pattern threshold, the method does not proceed any further and instead returns to step 510 for method 500 to repeat from the beginning. If the average rate of change of the series of temperature differences over the time period fall below the converging temperature pattern threshold, the method proceeds to step 582. In an example, the average rate of change may also be compared to the converging temperature pattern threshold at portions of the time period. The converging temperature pattern threshold may be a difference between the rates of change at the different portions of the time period. There may also be more than one converging temperature pattern threshold to determine the severity of the asymmetrical temperature pattern. For example, there may be a higher and lower converging temperature pattern threshold for detecting the risk level of the asymmetry, similar to Table 600 in FIG. 6 and the method for determining the risk level may be similar to method 700 in FIG. 7.

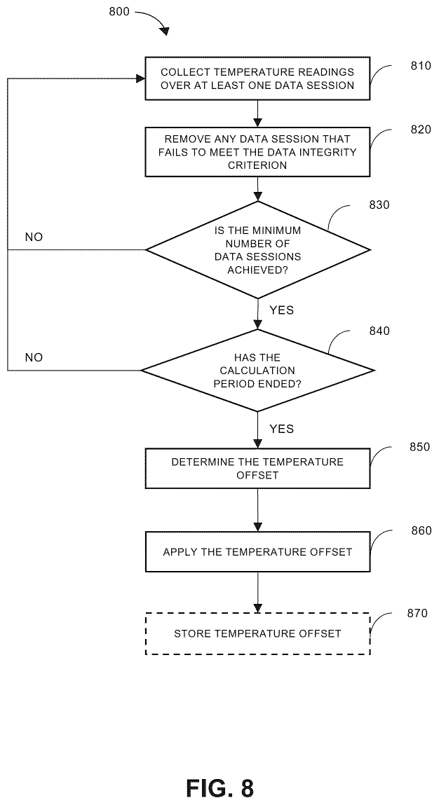

At step 581, a diverging asymmetrical foot temperature pattern flag is generated. The diverging asymmetrical foot temperature pattern flag may be presented as an alert. The alert may be sent to the user or may be sent to a third party. The diverging asymmetrical foot temperature pattern flag may indicate the need for a temperature offset, which may be determined using the average rate of change of the temperature differences (e.g. using method 800 in FIG. 8). The temperature offset may be used to counteract the creep of the temperature differences over the time period. For example, the temperature offset may include a temperature offset value that increases over the time period at a rate relative to the rate of change of the temperature differences. The resulting temperature differences may be more accurate for producing the foot health change flags in method 300. Alternatively, the diverging asymmetrical foot temperature pattern flag may be used to indicate an oncoming vascular disease presenting in one of the user's two feet. The diverging asymmetrical foot temperature pattern flag may be used to adjust the care actions or care level of the user.

At step 582, a converging asymmetrical foot temperature pattern flag is generated. The converging asymmetrical foot temperature pattern flag may be presented as alert. The alert may be sent to the user or may be sent to a third party. The converging asymmetrical foot temperature pattern flag may indicate the need for a temperature offset, which may be determined using the average rate of change of the temperature differences (e.g. using method 800 in FIG. 8). The temperature offset may be used to counteract the creep of the temperature differences over the time period. For example, the temperature offset may include a temperature offset value that decreases over the time period at a rate relative to the rate of change of the temperature differences. The resulting temperature differences may be more accurate for producing the foot health change flags in method 300. Alternatively, the converging asymmetrical foot temperature pattern flag may be used to indicate an oncoming vascular disease presenting in one of the user's two feet. The converging asymmetrical foot temperature pattern flag may be used to adjust the care actions or care level of the user.

Alternatively, or additionally, there may be a single asymmetrical foot temperature pattern flag, which is generated using a combination of standard deviation and range calculations of the temperature differences. The alternative method may use the same steps 510 to 550 from method 500 after which, the standard deviation of the series of temperature differences may be calculated, which indicates the level of spread of the temperature differences. The standard deviation may be compared to a standard deviation threshold. Additionally, the range of the series of temperature differences may be calculated by taking the difference between a highest temperature difference value and lowest temperature difference value over the time period. The difference between the highest temperature difference value and the lowest temperature difference value may be used to determine the variability of the temperature differences. The difference between the highest temperature difference value and the lowest temperature difference value is compared to a temperature difference threshold. In one example, a general asymmetrical foot temperature pattern flag is generated when the standard deviation exceeds the standard deviation threshold, and the range of the temperature differences exceeds the temperature difference threshold. The example requires both the standard deviation and range of the temperature differences to exceed the respective thresholds.

If one or more of the standard deviation and range of the temperature differences does not exceed the standard deviation threshold and temperature difference threshold, respectively, the method does not proceed any further and repeats from step 510. In another example, a general asymmetrical foot temperature pattern flag is generated when the standard deviation exceeds the standard deviation threshold, or the range of the temperature differences exceeds the temperature difference threshold. In this example, either or both of the standard deviation and range of the temperature differences exceed the respective thresholds. If neither the standard deviation or range of the temperature differences exceed the standard deviation threshold and temperature difference threshold, respectively, the method does not proceed any further and restarts from step 510.

In another example, the general asymmetrical foot temperature pattern flag may be determined using a correlation between the temperature sensors 110. The correlation may be determined from contralateral temperature comparisons or ipsilateral temperature comparisons. A symmetrical standard deviation plot (i.e. correlation=1 and slope-intercept=0) for the temperature differences may indicate a symmetrical foot temperature pattern. A deviation from the symmetrical standard deviation plot may indicate an asymmetrical foot temperature pattern, or biological variations that disturb the typical pattern at one or more foot locations. The asymmetrical foot temperature pattern may be determined by comparing the correlation to a correlation threshold. For example, a general asymmetrical foot temperature pattern flag may be generated when the temperature differences have a correlation of 0.8, which does not meet a correlation threshold of 0.9. The correlation may also be evaluated at portions of the time period. For example, the time period may be split into one-hour periods and the correlation may be evaluated for each full one-hour period of temperature readings or processed temperature data. A similar correlation for each one-hour period may indicate a symmetrical foot temperature pattern. Conversely, if any of the one-hour periods have a lower correlation than the other one-hour periods, the low correlation may indicate an asymmetrical foot temperature pattern. The difference in the correlations between the one-hour periods and an average over the entire time period may be used to generate the asymmetrical foot temperature pattern flag.

Referring now to FIG. 6, shown therein is an example table 600 of the risk levels corresponding to the foot health change flag generated in method 300 of FIG. 3. The risk levels correspond to a likelihood of the user developing the foot health change. For example, the foot health change may be a DFU, and the likelihood of developing a DFU may increase depending on two or more risk factors measured by the input device 100. Each generated foot health change flag may be sorted by risk level and a higher risk level may be associated with a higher likelihood of developing the foot health change at the indicated plantar location. A higher risk level may be associated with expedient care (e.g. care actions for high risk) and more intensive treatment method to avoid the complications associated with the foot health change.

The risk levels may vary according to risk factors associated with the foot health change flags. The risk factors may correspond to scalable values, which may be used to identify the categories for determining the risk level. In the example shown in FIG. 6, the risk factors include the intensity of the foot health change flag (“intensity”) and the duration of the foot health change flag (“duration”). The scalable value for intensity may be a value from the comparison of the average temperature difference to the foot health change threshold. For example, the intensity of the foot health change may be represented by the scalable value of the foot health change threshold that is exceeded to generate the foot health change flag. A higher foot health change threshold may indicate a higher intensity and a lower foot health change threshold may indicate a lower intensity. In other examples, the intensity of the foot health change flag may be represented by the value of exceedance of the foot health change threshold (i.e. from step 370 in method 300). The value of the exceedance may be determined by calculating the difference between the average temperature difference and the foot health change threshold. The value of the exceedance may be compared to a range of values, each range corresponding to a category (e.g. high exceedance and low exceedance). A higher exceedance may indicate a higher risk level and a lower exceedance may indicate a lower risk level.

In the example shown in FIG. 6, the scalable value of the duration is the evaluation time (i.e. the length of the time period). A first time period may be longer than a second time period, where the average of the temperature difference in step 360 of method 300 is evaluated over both time periods. The longer time period may indicate a longer duration and the shorter time period may indicate a shorter duration. In other examples, the scalable value for duration may be the length of time (e.g. minutes, hours, days, weeks, or months), during which the foot health change flag is presented. For example, method 300 may be repeated for each time period, and the generation of a foot health change flag may begin a running counter of the consecutive time periods the foot health change flag is present. A higher number of consecutive time periods may indicate a higher duration. In other examples, the risk level may depend on other risk factors and the table may have different axes and/or may have a greater number of types of foot health change flags. Another risk factor may include the combinations of types of temperature differences that lead to the foot health change flag. For example, a foot health change flag from a contralateral temperature difference may have a higher risk level than from an ipsilateral temperature difference. A foot health change flag from a combination of both contralateral and ipsilateral temperature differences may have the highest risk level.

The risk levels may be quantitative risk levels or qualitative risk levels. The quantitative risk level may be represented by sequential numbers in regular intervals, with increasing value (e.g. 1, 2, 3, and 4, where 4 indicates the highest risk level). A higher value may correspond to a higher risk level than a lower value. The qualitative risk level may be a relative term indicating the type of risk, such as, “low risk”, “medium risk”, or “high risk”.

Each cell of the table 600 may correspond to a type of foot health change flag (“type of flag”). The foot health change flags are categorized using the risk factor criteria to determine which type of flag the foot health change flag corresponds to. In some examples, two or more types of flags in the table may have different risk levels. In other examples, two or more different types of flags may have the same risk level. For example, a first foot health change flag may be determined for a time period of two days using a first foot health change threshold of 2.2 degrees Celsius. A second foot health change flag may be determined for a time period of three days using a second foot health change threshold of 2.0 degrees Celsius. Both the first foot health change flag and second foot health change flag may have the same risk level. The risk level of each type of flag may be assigned using previous knowledge of risk factors and the impact of each risk factor on the likelihood of development of the foot health change.

In the example shown in FIG. 6, the x-axis of table 600 is the duration risk factor 660, and the y-axis is the intensity risk factor 650. However, the x-axis and y-axis may be switched in other embodiments. For example, step 370 of method 300 may be evaluated over a first time period and a second time period. The first time period may be a longer duration than the second time period (e.g. the second time period may be at least one day and the first time period may be at least three days). In the example, the duration risk factor 660 includes the first time period and the second time period, with the arrow indicating an increase in duration. Step 370 of method 300 may also be evaluated against a first foot health change threshold and a second foot health change threshold. The first foot health change threshold may be larger than the second foot health change threshold (e.g. the first foot health change threshold may be 2.2 degrees Celsius, and the second foot health change threshold may be 2.0 degrees Celsius). In the example, the intensity risk factor 650 includes the first foot health change threshold and the second foot health change threshold, with the arrow indicating an increase in intensity.

In another example, foot health change flags with longer flag durations and higher foot health change thresholds have a higher risk level. Furthermore, the first time period may be longer in duration than the second time period and the first foot health change threshold may be larger than the second foot health change threshold. The first type of flag 610 may be a foot health change flag for a second time period (i.e. a shorter duration time period) and exceeding a second foot health change threshold (i.e. a smaller foot health change threshold). The first type of flag 610 may correspond to the lowest risk level (i.e. risk level “1”). The second type of flag 620 may be a foot health change flag for a second time period and exceeding a first foot health change threshold (i.e. a larger foot health change threshold). The second type of flag 620 may correspond to the second lowest risk level, which may be a quantitative risk level of “2”. The third type of flag 630 may be a foot health change flag for a first time period (i.e. a longer duration time period) and exceeding a second foot health change threshold. The third type of flag 630 may correspond to the same risk level as the second type of flag 620. The third type of flag 630 may also correspond to the second lowest risk level, which may be a quantitative risk level of “2”. Finally, the fourth type of flag 640 may be a foot health change flag for a first time period and exceeding a first threshold. The fourth type of flag 640 may correspond to the highest risk level, which may be a quantitative risk level of “3”. The highest risk level may correspond to a first foot health change flag evaluated over the first time period and may exceed the first foot health change threshold. It is appreciated that risk levels may correspond to the risk factors. In the example table 600, the first type of flag 610 and the second type of flag 620; and the third type of flag 630 and fourth type of flag 640 are evaluated using the same time periods. Similarly, table 600 shows the first type of flag 610 and the third type of flag 630; and the second type of flag 620 and fourth type of flag 640 exceed the same foot health change threshold. It is appreciated that quantitative or qualitative risk levels other than the risk levels indicated in the example table 600, may be used.

Referring now to FIG. 7, shown therein is an example method 700 of generating a risk level when detecting a foot health change similar to method 300 of FIG. 3, and determining the risk level of each foot health change flag. As described for method 300 of FIG. 3, the foot health changes may include a DFU, foot deformity, asymmetrical temperature pattern, and/or foot infection. Method 700 uses the risk levels and risk factors shown in table 600 in FIG. 6. The example risk factors for the method may include duration and intensity. Method 700 is an example method for how the types of flags may be determined and the risk level for each type of flag, any alteration to table 600 of FIG. 6 may also be reflected in method 700.

At step 710, a temperature reading is collected at each time point in a time period to yield a series of temperature readings. The temperature readings may include more than one series of temperature readings, such as temperature readings from a first time period or a second time period and from a first plantar location or a second plantar location. For example, the temperature readings may include four series of temperature readings. A first series of temperature readings may be generated from a first temperature reading collected at a first plantar location at each time point over a first time period. A second series of temperature readings may be generated from a second temperature reading collected at the first plantar location at each time point over a second time period. A third series of temperature readings may be generated from a third temperature reading collected at a second plantar location at each time point over the first time period. A fourth series of temperature readings may be generated from a fourth temperature reading collected at the second plantar location at each time point over the second time period. The temperature readings may be collected using input device 100.

At optional step 720, a temperature offset is determined. The temperature offset is used to adjust the temperature readings to increase accuracy of foot health change detection. The temperature offset may be determined using previous temperature readings from the user or historical temperature readings from the input device 100. The method for determining the temperature offset may vary depending on the type of temperature offset being applied. Examples of temperature offsets and methods of determining temperature offsets are described in FIG. 10.

At optional step 730, the temperature offset is applied to the first series of temperature readings, to the second series of temperature readings, to the third series of temperature readings, and to the fourth series of temperature readings. The temperature offset may be applied to the first series of temperature readings to generate a first series of offset temperature data. Additionally, the temperature offset may be applied to the second series of temperature readings to generate a second series of offset temperature data, applied to the third series of temperature readings to generate a third series of offset temperature data, and applied to the fourth series of temperature readings to generate a fourth series of offset temperature data. The temperature offset may be a single temperature offset, such as a single temperature offset applied to one or more series of temperature readings. The temperature offset may also include multiple temperature offsets applied together to one or more series of temperature readings. The temperature offset may further include a unique temperature offset applied to each series of temperature readings, separately, or the temperature offset may include the same temperature offset applied to each series of temperature readings.

At optional step 740, irrelevant offset temperature data is identified and removed from the first and second series of offset temperature data or by removing the corresponding temperature readings from the first and second series of temperature readings. The irrelevant offset temperature data may be identified using one or more thresholds as with step 340 of method 300. Examples of filters used to identify and remove irrelevant offset temperature data are described in FIG. 13. A first series of processed temperature data may be generated once the irrelevant offset temperature data is removed from the first series of offset temperature data. Similarly, a second series of processed temperature data may be generated once the irrelevant offset temperature data is removed from the second series of offset temperature data, a third series of processed temperature data may be generated once the irrelevant offset temperature data is removed from the third series of offset temperature data, and a fourth series of processed temperature data may be generated once the irrelevant offset temperature data is removed from the fourth series of offset temperature data. The irrelevant offset temperature data may be identified and removed from the first series of offset temperature data, the second series of offset temperature data, the third series of offset temperature data, and fourth series of offset temperature data, individually. Alternatively, the irrelevant offset temperature data may be identified for a single series of offset temperature data and removed from each of the series of offset temperature data. The irrelevant offset temperature data may be removed by replacing the offset temperature data points identified as irrelevant offset temperature data with an NaN value or missing value and ignored or may be replaced with a synthesized or estimated real temperature difference value using interpolation or some other method. Additional data preprocessing steps may be taken.

After step 740, the method may proceed to two different pathways, each ending with a different type of flag and risk level categorizations. The first pathway (steps 751, 761, 771, 781, 791, and 792) includes the types of flags for a first time period with a first duration. The second pathway (steps 752, 762, 772, 782, 793, and 794) includes the types of flags for a second time period with a second duration. In the example shown in FIG. 7, the first time period is larger (i.e. longer in duration) than the second time period. Foot health change flag detection may occur over both the first time period and over the second time period, if the required temperature readings are collected. For example, a first time period may be three days and the second time period may be one day and the foot health change flag may be calculated over the one-day period and over the three-day period when the input device 100 has collected at least three days of temperature readings.

At step 751, a temperature difference is determined by calculating the difference between the temperature readings at the first plantar location and the second plantar location for the first time period. Step 751 is the first step in the first pathway, which is described in sequence below. Similar to step 350 in method 300, the temperature difference may be ipsilateral (i.e. on the same foot of the user), contralateral (i.e. on opposite feet of the user), or some other type of temperature difference. Particularly, the first series of temperature readings is compared to the third series of temperature readings to determine a temperature difference for each time point in the first time period. In the examples including optional steps 720 to 740, the first series of processed temperature data is compared to the third series of processed temperature data for each time point in the first time period.

At step 761, an average temperature difference is calculated over the first time period. The average temperature difference may be calculated by summing the temperature differences from step 751 and dividing the total by the number of temperature differences collected over the first time period. The average temperature difference over the first time period is then compared to a second foot health change threshold in the next step 771. In a specific example, the second foot health change threshold is a lower intensity than the first foot health change threshold, however, it is appreciated that other examples may have a different risk factor or different threshold value.

At step 771, the average temperature difference over the first time period is compared to the second foot health change threshold. If the second foot health change threshold is exceeded, the method proceeds to step 781. The exceedance of either of the first foot health change threshold or the second foot health change threshold results in a first foot health change flag. The following step 781 is used to determine the type of flag and corresponding risk level of the first foot health change flag. If the average temperature difference does not exceed the second foot health change threshold, then no foot health change flag is generated. Instead, the method proceeds back to step 710 to continue collecting temperature readings and determine if any new foot health change flags are present.

At step 781, the average temperature difference over the first time period is compared to the first foot health change threshold. If the average temperature difference does not exceed the first foot health change threshold, method 700 proceeds to step 791. In this case, the first foot health change flag exceeds the second foot health change threshold (i.e. the lower intensity threshold), but does not exceed the first foot health change threshold (i.e. the higher intensity threshold). The lower foot health change threshold is used to determine the type of flag and the corresponding risk level for the type of flag. If the average temperature difference exceeds the first foot health change threshold, method 700 proceeds to step 792. In this case, the foot health change flag exceeds both the second foot health change threshold and the first foot health change threshold. The higher intensity foot health change threshold is used to determine the type of flag and the corresponding risk level for the type of flag.

At step 791, the first foot health change flag is generated with a risk level corresponding to the third type of flag 630. The third type of flag 630 may correspond to a risk level of “2” or the second lowest risk level. The third type of flag 630 may be for a longer time period and a smaller (i.e. lower intensity) foot health change threshold, as shown in table 600 of FIG. 6.

At step 792, the first foot health change flag is generated with a risk level corresponding to the fourth type of flag 640. The fourth type of flag 640 may correspond to a risk level of “3”, which may be the highest risk level. The fourth type of flag 640 may be for a shorter time period and a larger (i.e. higher intensity) foot health change threshold, as shown in table 600 of FIG. 6.

At step 752, a temperature difference is determined by calculating the difference between the temperature readings at the first plantar location and the second plantar location for the second time period. Step 752 is the first step in the second pathway, which is described in sequence below. Similar to step 350 in method 300, the temperature difference may be ipsilateral (i.e. on the same foot of the user), contralateral (i.e. on opposite feet of the user), or some other type of temperature difference. Particularly, the second series of temperature readings is compared to the fourth series of temperature readings to determine a temperature difference for each time point in the second time period. In the examples including optional steps 720 to 740, the second series of processed temperature data is compared to the fourth series of processed temperature data for each time point in the second time period.

At step 762, an average temperature difference is calculated over the second time period. The average temperature difference may be calculated by summing the temperature differences from step 752 and dividing the total by the number of temperature differences collected over the second time period. The average temperature difference over the second time period is then compared to a second foot health change threshold in the next step 772.

At step 772, the average temperature difference over the second time period is compared to the second foot health change threshold. If the second foot health change threshold is exceeded, the method proceeds to step 782. The exceedance of either of the first foot health change threshold or the second foot health change threshold results in a second foot health change flag. The following step 781 is used to determine the risk level corresponding to the second foot health change flag. If the average temperature difference does not exceed the second foot health change threshold, then no foot health change flag is generated. Instead, the method continues back to step 710 to continue collecting temperature readings and determine if any new foot health change flags are present.

At step 782, the average temperature difference over the second time period is compared to the first foot health change threshold to determine the risk level corresponding to the second foot health change flag. If the average temperature difference does not exceed the first foot health change, method 700 proceeds to step 793. In this case, the foot health change flag exceeds the second foot health change threshold (i.e. the lower intensity threshold), but does not exceed the first foot health change threshold (i.e. the higher intensity threshold). The second foot health change threshold category is used to determine the type of flag and the corresponding risk level for the type of flag. If the average temperature difference exceeds the first foot health change, method 700 proceeds to step 794. In this case, the foot health change flag exceeds both the second foot health change threshold and the first foot health change threshold. The higher intensity foot health change threshold is used to determine the type of flag and the corresponding risk level for the type of flag for the second foot health change flag.

At step 793, the second foot health change flag is generated with a risk level corresponding to the first type of flag 610. The first type of flag 610 may correspond to a risk level of “1”, which may be the lowest risk level. The first type of flag 610 may be for a shorter time period and a smaller (i.e. lower intensity) foot health change threshold, as shown in Table 600 of FIG. 6.

At step 794, the second foot health change flag is generated with a risk level corresponding to the second type of flag 620. The second type of flag 620 may correspond to a risk level of “2”, which may be the same risk level as the third type of flag 630. In alternative examples, the second type of flag 620 and the third type of flag 630 may have different risk levels. The second type of flag 620 may be for a shorter time period and a larger (i.e. higher intensity) foot health change threshold, as shown in Table 600 of FIG. 6.

Method 700 may be used to compare the first foot health change flag to the second foot health change flag. For example, the highest risk level may correspond to the first foot health change flag when the first foot health change flag exceeds the first foot health change threshold (i.e. higher intensity). As described above, the first foot health change flag may be evaluated over the first time period, which is longer in duration than the second time period. In the example, the first foot health change flag is a higher risk than the second foot health change flag, which includes any foot health change flag calculated over the second time period. The combination of a higher intensity threshold and longer time period results in a fourth type of flag 640 of FIG. 6, which may be the highest risk level. In another example, the first time period and the second time period may be the same time period, the first foot health change flag may exceed the first foot health change threshold, and the second foot health change flag may only exceed the second foot health change threshold. The first foot health change flag may correspond to a higher risk level than the second foot health change flag because the first foot health change flag exceeds a higher intensity threshold. Similarly, the first foot health change threshold and the second foot health change threshold may have the same value, the first foot health change flag may be evaluated over the first time period, and the second foot health change flag may be evaluated over the second time period. The first foot health change flag may correspond to a higher risk level because the first foot health change flag is evaluated over a longer duration time period.

Referring now to FIG. 8, shown therein is example method 800 for determining the temperature offset in step 320 of method 300 in FIG. 3. Method 800 may also be used to determine the temperature offset in optional steps 420 in FIG. 4, 520 in FIG. 5, and 720 in FIG. 7. The temperature offset may be used to adjust the temperature readings collected in method 300, 400, 500, and 700 to improve the accuracy of the foot health change detection. Method 800 may be completed using previous temperature readings or historical temperature readings collected by input device 100. The previous or historical temperature readings may be stored in the memory or storage of the input device 100 and retrieved to determine the temperature offset. The temperature offset for each temperature sensor in the input device 100 may be initialized with a value of zero before the first temperature offset calculation. Method 800 may then be used to calculate a first version of the temperature offset and replace the zero value with the calculated value. The temperature offset may be updated using method 900.

The temperature readings over the time period may be separated into at least one data session. The data session may be a portion of the time period. Each data session has a designated starting point and an ending point, both of which are included within the time period. The time period may be separated into the at least one data session by detecting events for the starting and ending points of each data session and segregating the temperature readings within the starting and ending points. The segregated temperature readings may be considered part of a data session. In a specific example, a data session may include the temperature readings (e.g. the first series of temperature readings and the second series of temperature readings) starting when the input device 100 is donned by the user and ending when the input device 100 is doffed by the user and enters sleep mode. The time period may be three days and the user may have six data sessions wherein the user dons the input device 100 and doffs the input device 100 twice, for each of the days. In other examples, the starting and ending point may be sets of time. Each data session may include temperature readings starting when the first temperature reading is detected and ending when one hour of temperature readings have been collected after the input device 100 enters sleep mode. Sleep mode may be an input device 100 function for ceasing temperature reading collection when the input device 100 no longer detects motion. The motion may be detected using the optional pressure sensors 116 (i.e. lack of pressure corresponds to lack of motion) or the optional IMU 122. The input device 100 may be continuously checked for a change in motion conditions to determine when sleep mode begins and ends. In one example, the input device 100 may enter sleep mode after 30 minutes of no motion and exit sleep mode when motion is first detected.

At step 810, temperature readings are collected over at least one data session. The temperature readings may be collected by the temperature sensors 110 in the input device 100. Alternatively, an external device such as a thermogram or temperature mat may be used to collect the temperature readings. The temperature readings from the external device may be different than the temperature readings collected by the input device 100 (e.g. the external device may have more granular temperature detection). Additionally, the temperature readings from the external device may be collected when the input device 100 is not worn by the user. For a data session recorded with an external device, the data session may start when the temperature readings start being collected and end when the external device stops collecting data. The data session for an external device may be shorter or longer than a data session for an input device 100. The processor 120 or external processor may determine which temperature readings belong to the at least one data session. Additionally, the processor 120 or external processor may sort the temperature readings into each data session.

At step 820, the at least one data session is compared to data integrity criterion. Each of the data sessions in the at least one data session (e.g. each data session when the at least one data session includes more than one data session) may be required to meet data integrity criterion before being used to calculate the temperature offset. Failing to meet the data integrity criterion may include detecting a data session duration below a duration threshold. For example, a data session may fail to meet the data integrity criterion when the time period of temperature readings is less than a duration threshold of at least 15 minutes. More than one data integrity criterion may be applied to the data sessions. Failing to meet the data integrity criterion may also include detecting a data connection issue. For example, the at least one data session may be discarded when the difference between the time point of the first temperature reading from the left input device and the second temperature reading from the right input device is greater than 10 minutes. A larger difference value between the left and right input device may indicate a connection issue in one of the input devices, which may result in inaccurate temperature differences and an inaccurate foot health change flag. The left and right input device may also be evaluated to determine if external factors may be causing inaccurate temperature readings between the left and right input device. One external factor that may cause a failure to meet the data integrity criterion may include detecting wireless charger heat for an input device 100 containing a wirelessly rechargeable battery and wireless charging receiver 126. During charging, the wireless charging receiver 126 may emit heat, which artificially inflates the temperature readings of any nearby temperature sensors 110. The data session for the entire input device 100 or specific temperature sensors 110 in proximity to the wireless charging receiver 126 may fail to meet the data integrity criterion when wireless charger heat is detected. The wireless charging heat may be detected using a set threshold or a calculated threshold. For example, wireless charger heat may be detected when the temperature readings from the temperature sensors 110 in proximity to the wireless charging receiver 126 exceed a set threshold of 30 degrees Celsius. In another example, the wireless charger heat may be detected when the temperature readings from the temperature sensors 110 in proximity to the wireless charging receiver 126 exceed a calculation of the mean temperature data from temperature sensors 110 not in proximity to the wireless charging receiver, over the time period. If a single temperature reading or number of temperature readings exceed the set threshold or calculated threshold, the data session may fail to meet the data integrity criterion.

At step 830, the processor 120 or external processor determines if the required number of data sessions are achieved. The data sessions may then be retrieved from the storage or memory to complete the temperature offset calculation. Data sessions that do not meet the data integrity criterion in step 820 may not be counted as part of the at least one data session. For example, the temperature offset may be calculated when a minimum of five data sessions is collected. If the minimum number of data sessions is achieved, the method proceeds to step 840. If the minimum number of data sessions is not achieved, the method does not proceed any further and instead returns to step 810. The data sessions may be stored until a required number of data sessions are achieved. The method may continue collecting temperature readings over new data sessions before repeating method 800 with a greater number of data sessions. During step 830, the new data sessions may be collected as the user continues to wear the input device 100 and added to the existing number of data sessions. The total number of data sessions may be increased until the number of data sessions exceeds the minimum number of data sessions.

At step 840, it is determined if the calculation period has ended. The calculation period is the duration of time required before the temperature offset can be determined. If the calculation period has not ended, the method proceeds to step 810. In a specific example, the calculation period may be two weeks and the temperature offset may not be determined when less than two weeks of calculation period has passed. The calculation period may include more than one time period of temperature reading collection. The time periods of temperature reading collection may be stored in the memory or storage. If the calculation period has ended, the method proceeds to step 850.

At step 850, the temperature offset is determined using the temperature readings from the at least one data session that did not fail to meet the data integrity criterion. The temperature offset may be calculated for each temperature sensor in the input device 100, individually, or may be calculated for the entire input device 100 using a single series of temperature readings. For example, the temperature offset for a first temperature sensor 110a may be applied to the series of temperature readings recorded by the first temperature sensor 110a. Similarly, the temperature offset for the second temperature sensor 110b may be applied to the series of temperature readings recorded by the second temperature sensor 110b. Alternatively, the same temperature offset may be applied to the temperature readings from both the first temperature sensor 110a and the second temperature sensor 110b.

At step 860, the temperature offset is applied to the temperature readings, as indicated in step 330 in method 300.

At optional step 870, the temperature offset is stored in the memory or storage. The temperature offset may be stored and later applied to the temperature readings. The temperature offset may be stored in the form of a table or a list with a temperature offset for each associated temperature sensor in the input device 100. For example, five temperature offsets may be determined for each temperature sensor in a temperature sensor array 108 with five temperature sensors 110. The temperature offset may also be determined for all of the temperature sensors 110 at once. For example, a single temperature offset may be applied to each of the temperature sensors 110.

In some examples, the temperature offset may only be calculated once and maintained for the lifetime of the input device 100. Method 800 may only be completed once, and the temperature offset may be used in every repetition of step 330 of method 300. In other examples, the temperature offset may be updated to improve the accuracy of the temperature offset over time. The updating of the temperature offset may be completed using method 900, described below.

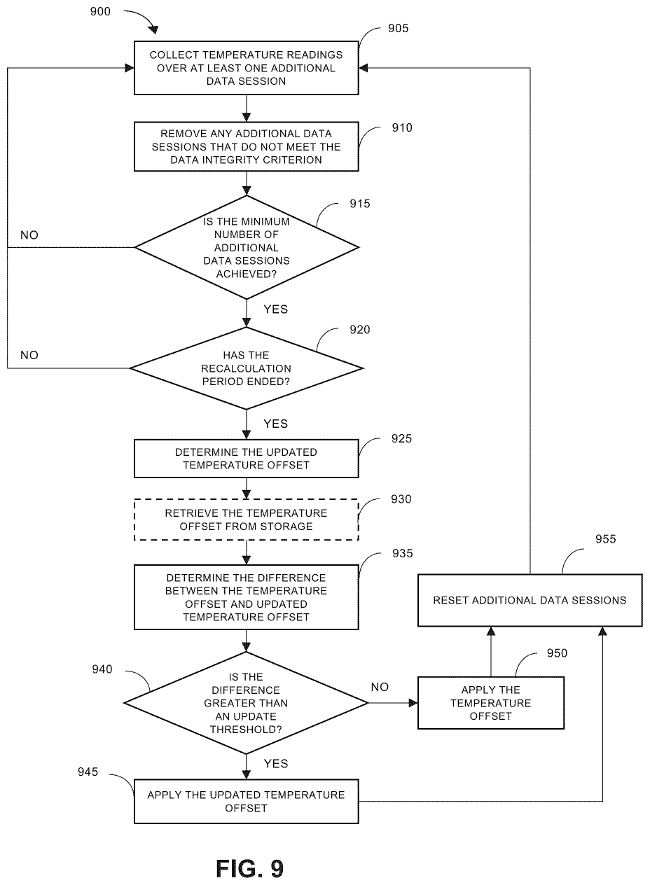

Referring now to FIG. 9, shown therein is an optional method for updating the temperature offset. The updated temperature offset may be determined after the temperature offset has been obtained using method 800 in FIG. 8. The updated temperature offset may be generated using at least one additional data session, which is collected during an update period. The updated temperature offset may be determined for one or more of the temperature sensors 110. The temperature offset may also be updated at set times. The update may occur every set number of minutes, hours, days, or weeks after the initial temperature offset calculation. For example, method 900 may occur every two weeks. Method 900 may also be used to update the temperature offset in step 320 in FIG. 3 and in optional steps 420 in FIG. 4, 520 in FIG. 5, and 720 in FIG. 7. Method 900 may be initiated by a care action after the foot health change flag is generated. The care action may include, for example, an input device 100 modification, amputation, an assessment, an intervention, imaging, diagnostic tests, physical exams, prescription of an assistive device, a vascular procedure, medication, pharmacological therapy, activity/movement prescription, or the cessation or reversal of a care action. The temperature offset may be adjusted or replaced with the updated temperature offset determined using method 900.

At step 905, temperature readings are collected over at least one additional data session. The additional data session may occur after method 800 is completed. The update period may consecutively or non-consecutively follow the calculation period or some other update period. The temperature readings may also be collected by the temperature sensors 110 in the input device 100.

At step 910, the at least one additional data session is compared to the data integrity criterion. Each of the data sessions in the at least one additional data session may be required to meet the data integrity criterion before being used to update the temperature offset. Failing to meet the data integrity criterion may include one of detecting a data session duration below a duration threshold, detecting a data connection issue, and detecting wireless charger heat, as described in step 820 of method 800.

At step 915, the processor 120 or external processor determines if the required number of additional data sessions are achieved. As with data sessions in method 900, the at least one additional data session may be stored until a minimum number of additional data sessions are achieved. The at least one additional data session may then be retrieved from the storage or memory to complete the temperature offset update. For example, the updated temperature offset may be determined from a minimum of five additional data sessions. The updated temperature offset may be determined once the minimum number of additional data sessions are achieved, and the method proceeds to step 920. If the minimum number of additional data sessions have not been achieved, the method does not proceed any further and instead returns to step 905. During step 915, more additional data sessions may be collected and added to the existing number of additional data sessions. The total number of additional data sessions may be increased until the number of additional data sessions exceeds the minimum number of additional data sessions.

At step 920, the processor 120 or external processor determines if the update period has ended. The update period is the duration of time required before the updated temperature offset can be calculated. The update period may be a different duration than the calculation period and may include one or more time periods. For example, the update period may include the calculation period. The calculation period may be two weeks starting on a first day and the update period may be four weeks starting on the first day. In the example, the update period includes the two weeks of temperature readings collected during the calculation period and the additional two weeks of temperature readings collected after the end of the calculation period. The update period may also be the same duration as the calculation period. For example, the calculation period and the update period may both be two weeks in duration. If the update period has not ended, the method does not proceed any further. Instead, the method is repeated from step 905. For example, the update period may be two weeks and the updated temperature offset may not be determined when one week has passed. If the update period has ended, the method proceeds to step 925. The update period may also be triggered based on a specific event, such as when an external device is collecting temperature readings. For example, the update period may occur when new temperature readings are collected from the external device.

At step 925, the updated temperature offset is determined using the temperature readings from the at least one additional data session that did not fail to meet the data integrity criterion. As with step 850 in method 800, the updated temperature offset may be determined for each temperature sensor in the input device 100, individually, or may be calculated for every temperature sensor in the input device 100, collectively.

At optional step 930, the temperature offset may be retrieved from the storage or memory. Each temperature offset and updated temperature offset may be stored in the storage or memory to be retrieved and used in any further calculations.

At step 935, the difference between the temperature offset and the updated temperature offset is determined. The temperature offset may be the most recent temperature offset. For example, the temperature offset may be from the time period prior to the current time period. Alternatively, the temperature offset may be from a non-consecutive time period, such as, a first time period for the user or the last used time period. The first time period for the user may include the first collected temperature readings from the user when the user wore the input device 100 for the first time. The last used time period may include the temperature readings collected during the time period used to calculate the last applied temperature offset. The difference between the temperature offset and the updated temperature offset shows the change in the temperature offsets between the chosen time periods.

At step 940, the processor 120 or external processor determines if the difference between the temperature offset, and the updated temperature offset exceeds an update threshold. The update threshold may be a set value. For example, the update threshold may be 1 degree Celsius. The difference is compared to the update threshold to determine whether the temperature offset, or the updated temperature offset is applied to the temperature readings. If the difference between the temperature offset and the updated temperature offset exceeds the update threshold, the method proceeds to step 945. If the difference between the temperature offset and the updated temperature offset does not exceed the update threshold, the method proceeds to step 950.

At step 945, the updated temperature offset is applied to the temperature readings as part of step 330 in method 300. The updated temperature offset may be applied when the change in temperature offset is large because the updated temperature offset may be more accurate for the user's most recent conditions. For example, the updated temperature offset may include additional data that more accurately represents the user's natural physiological foot temperature difference. The updated temperature offset may be a personalized left-to-right offset applied to increase the accuracy of the resulting foot health change flags in method 300. In another example, the updated temperature offset may be an updated technology offset. The technology offset may be determined over the update period to detect any changes in the drift of the temperature sensors 110 over time. The updated technology offset may be applied when the sensor drift results in a difference between the previous technology offset and the updated technology offset that exceeds the update threshold.

At step 950, the temperature offset is applied to the temperature readings as part of step 330 in method 300. The temperature offset may be a previously calculated temperature offset. The temperature offset may be applied because the change in the difference between the temperature offset and the updated temperature offset is not large enough to increase or decrease the change to the temperature readings. A smaller change may indicate regular variance in physiological or technological temperature sensor conditions, instead of an increase in accuracy. The previously calculated temperature offset may be maintained and applied to the temperature readings to prevent high fluctuation temperature offsets that may cause inaccurate variations in the number, intensity, or duration of foot health change flags.

At step 955, the additional data sessions are reset. The additional sessions may be counted using a continuous counter with the number of additional data sessions increasing when the temperature readings from each new additional data session are collected. The counter for the additional data sessions may be reset and the temperature readings from the additional data sessions may also be reset before method 900 can be repeated.

Referring now to FIG. 10, shown therein is a diagram of an offset module 1000 containing the various temperature offsets that may be applied as part of method 300, or optionally applied as part of methods 400, 500, and 700. The temperature offsets are applied to the temperature readings taken from the temperature sensors 110 in the input device 100. Temperature offsets are used to adjust the raw or processed temperature readings based on personal, population, or technology considerations that may cause inaccurate foot health change flags. The temperature offset may be calculated when the input device 100 is initially created, or the temperature offset may be calculated when the input device 100 is worn by the user. Each of the temperature offsets in the offset module 1000 may be determined and applied to the temperature readings as part of step 320 and 330 of method 300, and optional steps 420 and 430 of method 400, 520 and 530 of method 500, and 720 and 730 of method 700, respectively. One or more of the temperature offsets in the offset module 1000 may be applied to the temperature readings.

In some examples, the temperature offset for the series of temperature readings may be zero. The temperature offset may be determined using method 800 in FIG. 8. However, the temperature offset may be zero for one or more of the types of temperature offsets. For example, the user may not have a natural temperature offset between the left and right foot. If a temperature offset includes determining a personalized left-to-right offset, the temperature offset may be zero. The temperature offset of zero is applied to the temperature readings and will not change the values of the temperature readings.

The temperature offsets may be constants. The constants may be a numerical integer or rational number, which is added or subtracted from the temperature readings in various methods used to determine the foot health change flags. The temperature offset for each temperature sensor may be stored in a table linking the temperature offset to the respective temperature sensor. The temperature offset may be applied to the temperature readings from the temperature sensor in the table. In other examples, the temperature offsets may be multipliers. For example, the temperature readings at a first temperature sensor 110a may be scaled by the multiplier determined for the first temperature sensor 110a. In cases where more than one temperature offset is applied, the temperature offsets may be compounded. For example, a first temperature offset may be a technology offset and a second temperature offset may be a personalized left-to-right offset. The first temperature offset may be a value of three for the heel temperature sensor and the second temperature offset may be a value of two for the heel temperature sensor. The first temperature offset and the second temperature offset may be added together to create a compounded temperature offset of five, which may be added to each of the temperature readings from the heel temperature sensor.

The first type of temperature offset that may be applied is a personalized temperature offset 1010. Personalized temperature offsets 1010 are unique to the user and require temperature readings from the user. The personalized temperature offsets 1010 may be determined using previous temperature readings from the current time period and/or from previous time periods. For example, the temperature offset for the series of temperature readings may be an average of previous temperature readings for the user (e.g. five days of previous temperature readings). The previous time periods may include time periods when the user does not have any foot health changes. The user, remote patient monitor, and/or health care provider may provide confirmation that the user does not have a foot health change during the previous time periods before the temperature data from the previous time periods are used to determine the temperature offset.

The first personalized temperature offset 1010 that may be applied is a personalized left-to-right offset. The personalized left-to-right offset captures any biological temperature differences between the opposite feet of the user. The personalized left-to-right offset may be applied to contralateral temperature differences between the user's feet. The naturally occurring biological temperature differences may cause inflated temperature difference calculations, resulting in a false foot health change flag. The personalized left-to-right offset may be applied to bring the temperature readings to an equivalent baseline for contralateral temperature difference calculations. The personalized left-to right offset may be used to improve the accuracy of foot health change detection. Another type of personalized temperature offset 1010 may include a personalized gradient offset. The personalized gradient offset includes adjustments to temperature readings based on the natural spatial intra-foot temperature gradient of the first foot of the user or the second foot of the user. The personalized gradient offset is similar to the personalized left-to-right offset, except the personalized gradient offset is applied to increase the accuracy of ipsilateral temperature differences. The ipsilateral temperature differences may include comparing two or more temperature readings for temperature sensors 110 at two different plantar locations on the same foot. The naturally occurring biological temperature differences across the user's foot (e.g. higher temperature at the heel than at the toes, or higher temperature in one angiosome as compared to another angiosome due to microvascular disease, macrovascular disease, or both) may cause inflated temperature difference calculations, resulting in a false foot health change flag. The personalized gradient offset may be applied to bring the temperature readings to an equivalent baseline for ipsilateral temperature difference calculations, to ensure only true foot health changes are flagged.

For personalized offsets, the data sessions may be recorded when the input device 100 is being worn by the user and the temperature sensors 110 are at equilibrium with the temperature of the user's foot or feet. The previous temperature readings may be recorded at a set time in the day or a set time after the first time point in the series of time points to improve the accuracy of the personalized offset calculation. Specifically, the temperature readings may be collected at a time when the user is not exposed to any external factors that may alter the temperature readings. For example, the temperature readings may be taken just after the input device 100 reaches equilibrium with the user's foot (e.g. thirty minutes after the start of a data session). Also, the temperature readings may only be taken when the user is at rest to avoid artificially inflated foot temperature values resulting from physical activity.

Another temperature offset that may be applied is a population offset 1020. The population offset 1020 may include population left-to-right temperature differences and population gradient temperature differences. The population left-to-right temperature differences may be applied to, for example, contralateral temperature difference calculations. The population gradient temperature differences may be applied to, for example, ipsilateral temperature difference calculations. The population offset 1020 may be calculated using temperature readings collected from a group of users. The group of users may represent a population of users. For example, the temperature readings for the population offset 1020 may be taken from a group of users with varying demographic, anatomical, and physiological differences. The group of users may include the user or may not include the user. The temperature readings are recorded for one or more data sessions from each user in the group of users. The contralateral temperature differences, ipsilateral temperature differences, or other temperature differences may be recorded for each temperature sensor 110. The temperature differences may be averaged or input into a weighted calculation to determine an overall population offset value for each temperature sensor 110 based on the group of users.

The population offset 1020 temperature readings may be collected when the input device 100 is positionable on the foot of the group of users. For example, each user in the group of users may wear the input device 100 for one or more time periods, during which temperature readings are collected. The collected temperature readings may then be recorded in the memory or storage before processing. During processing, the population offset 1020 may be calculated using the temperature readings for each of the users in the group of users. The population offset 1020 may be applied to counteract the average natural temperature gradients and left-to-right biases for the group of users. In some examples, the population offset may be determined using the temperature readings from every user in the group of users. Alternatively, the population offset 1020 may be determined using a portion of the users in the group of users. Demographics, measurements and/or statistics of each user may be recorded and inputted into the processor 120 with the temperature readings for each user in the group of users. The demographics, physical attributes, or health statistics may be used to pull temperature readings from individuals in the group of users who match the demographics, physical attributes, and/or health statistics of the user. Using a portion of the group of users may increase the accuracy of the population offset 1020. For example, the population offset 1020 for a user with peripheral neuropathy may be calculated using temperature readings for users in the group of users with peripheral neuropathy. The temperature readings may be taken in a controlled environment, such as a lab or may be taken at set times during the day or set times in the time period. In other examples, the temperature readings may be taken by an external device (e.g. infrared camera or thermogram). The conditions during which temperature readings are collected from an external device may be controlled.

The population offset 1020 may be updated at regular intervals when additional population data is obtained. For example, temperature readings may be continually collected for the group of users and the population offset 1020 may be updated when the minimum number of sessions in the update period is achieved. The method for updating the population offset 1020 may be the same as method 900 in FIG. 9, except the temperature readings are collected from a group of users instead of a single user.

Another temperature offset may include a technology offset 1030. The technology offset 1030 may be used to adjust temperature readings based on the technological electrical differences (e.g. resistance) of the temperature sensors 110 in the input device 100. The technology offset may be applied using contralateral comparisons (i.e. left-to-right foot) or using ipsilateral comparisons (i.e. same foot). The technology offset 1030 may be used as a method for calibrating the temperature sensors 110. The calibration may be completed before method 300 in FIG. 3 is carried out to improve the accuracy of the temperature readings.

For the technology offset 1030, the data sessions may be recorded when the input device 100 is not worn by the user or not reliably adjusted to foot temperatures and is instead adjusted to room temperature. In some cases, the technology offset 1030 may only be determined when the input device 100 is at a known temperature (e.g. room temperature). The known temperature may be determined using an ambient temperature sensor or by placing the input device 100 in a contained space with a known temperature (e.g. a temperature bath). The temperature readings may only be recorded during designated times when the input device 100 is not usually worn, to ensure the temperature readings are only taken when the input device 100 is not worn or not reliably adjusted to foot temperatures. For example, the temperature readings used may only be collected from midnight to 5:00 AM because the user may be sleeping and therefore, not wearing the input device 100. Alternatively, a pattern recognition algorithm or machine learning model may be used to determine the daily habits of the user and detect the daily times when the user does not wear the input device 100. The temperature reading times may then be selected using the pattern recognition algorithm. In another example, the temperature readings may only be recorded at the start of a data session when the user wakes the input device 100 from sleep mode. The first temperature readings from the input device 100 may occur when the temperature sensors 110 have not adjusted to the temperature of the user's foot and instead are adjusted to room temperature.

The technology offset 1030 may be updated at regular intervals to adapt to any temperature sensor drift or other sensor technology changes over time. For example, sensor drift may cause the temperature sensors 110 to increase in temperature values as the input device gets older. The use of the updated technology offset 1030 may be used to continuously recalibrate the temperature readings to room temperature and counteract the rise of the temperature readings over time. The update method may be the same as method 900 of FIG. 9. The update period may be shorter than for the personalized temperature offset 1010 and the population temperature offset 1020.

Another temperature offset may be determined using an activity classification algorithm or predictive algorithm for the user's daily patterns. An example of an activity classification algorithm that may be used to classify sensor data is described in U.S. Pat. No. 11,526,749 entitled “METHOD AND SYSTEM FOR ACTIVITY CLASSIFICATION”, the entirety of which is incorporated herein by reference. The temperature offset may be determined by recognizing various user activities and adjusting the temperature offset based on the type of activity. The activities that may be identified using the activity classification algorithm include donning the input device 100, doffing the input device 100, and high intensity activities (e.g. walking, running, jumping, etc.). The activity classification algorithm may be used, for example, to recognize when the user is running and determine a temperature offset that adjusts the temperature readings to account for excessive heat from intense physical activity. Similarly, the temperature offset may also be adjusted based on a predictive model that uses previous daily activities from the user to determine any future daily activities. The temperature offset may be determined based on the type of daily activity and the effect of the daily activity on the plantar temperatures of the user.

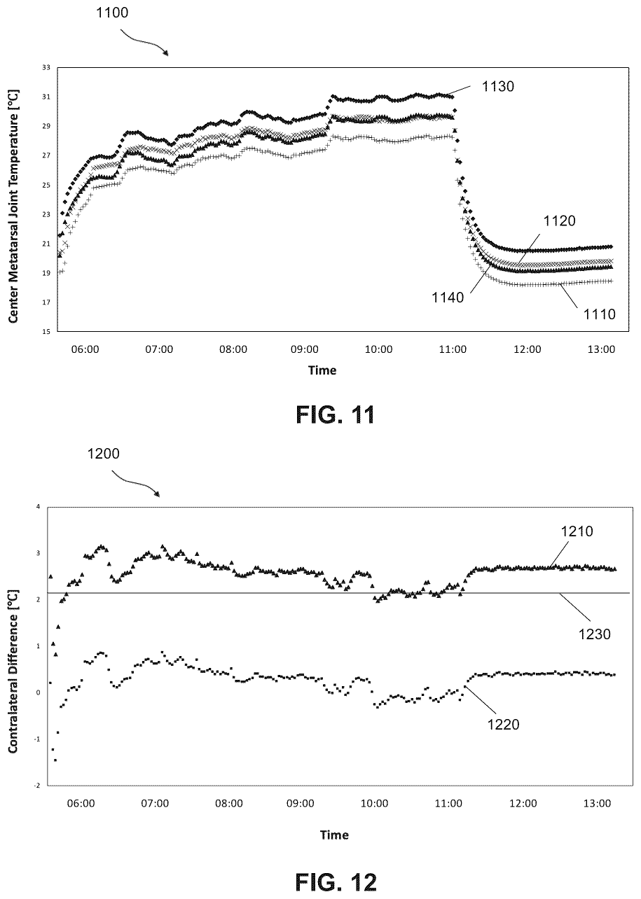

Referring now to FIG. 11 and FIG. 12, shown therein is an example embodiment of the applied temperature offset from method 300 in FIG. 3. FIG. 11 shows the raw temperature readings from the temperature sensors 110 in the input device 100. The offset temperature graph 1100 shows the temperature readings from the input device 100 with a temperature sensor under the left center metatarsal joint and a temperature sensor under the right center metatarsal joint. FIG. 11 shows the first series of temperature readings 1110 from a left center metatarsal joint temperature sensor, the first series of offset temperature data 1120 from the left center metatarsal joint temperature sensor readings after the temperature offset is applied, the second series of temperature readings 1130 from a right center metatarsal joint temperature sensor, and the second series of offset temperature data 1140 from the right center metatarsal joint temperature sensor readings after the temperature offset is applied. The vertical axis shows the temperature values, and the horizontal axis shows the time. The time may be the local time for the time zone the input device 100 and the user are located. In the example, the temperature is in degrees Celsius. It is appreciated that the temperature and time may be reported in other units. FIG. 12 shows a contralateral temperature difference graph 1200. The contralateral temperature difference graph 1200 includes a series of contralateral temperature difference readings 1210 calculated by taking the difference between the first series of temperature readings 1110 and the second series of temperature readings 1130 of FIG. 11 (i.e. subtracting the first series of temperature readings 1110 from the second series of temperature readings 1130). The foot health change threshold line 1230 is at an example value of 2.2 degrees Celsius. The embodiment shows a majority of the contralateral temperature difference readings 1210 exceed the foot health change threshold line 1230, which results in an inaccurate foot health change flag (e.g. generated at step 380 of method 300). The series of contralateral offset temperature difference readings 1220 show the difference between the first series of offset temperature data 1120 and the second series of offset temperature data 1140 from FIG. 11 (i.e. subtracting the first series of offset temperature data 1120 from the first series of offset temperature data 1140). After the temperature offset is applied, the majority of the series of contralateral offset temperature difference readings 1220 are below the foot health change threshold line 1230, which does not result in a foot health change flag. The temperature offset brings the first series of temperature readings 1110 and the second series of temperature readings 1130 to a closer baseline, which may improve the accuracy of the foot health change flag detection.

Now referring to FIG. 13, shown therein is a list of example irrelevant offset temperature data that may be identified and removed. The irrelevant offset temperature data module 1300 may include a variety of filters to apply to the offset temperature data at step 340 of FIG. 300 and at optional steps 440 of FIG. 400, 540 of FIG. 500, and 740 of FIG. 7. The irrelevant offset temperature data may be identified and removed from the series of offset temperature data. The irrelevant offset temperature data includes offset temperature data that may correspond to events that alter the integrity of the temperature readings. The events may still be evident after the temperature offset is applied and are subsequently identified and removed to prevent the calculation of inaccurate temperature differences, which may affect the accuracy of the generation of the foot health change flag. In some examples, one or more filters may be applied, and zero data points are identified and removed as irrelevant offset temperature data.

The first filter in the irrelevant offset temperature data module 1300 may be a partially broken sensor filter 1310, which may identify and remove offset temperature data indicating a partially broken temperature sensor. Partially broken temperature sensors may generate sporadic and/or noisy offset temperature data. A partially broken sensor may be detected using a variance calculation within the time period, such as the standard deviation and/or an offset temperature difference for each temperature sensor. In a first example, the standard deviation may be calculated for the series of offset temperature data over the time period. The standard deviation may be compared to a standard deviation threshold and the offset temperature data for the temperature sensor may be identified as irrelevant offset temperature data if the standard deviation over the time period exceeds the standard deviation threshold. The entirety of the series of offset temperature data may be removed when the standard deviation over the time period exceeds the standard deviation threshold. For example, the standard deviation threshold may be 10 and the time period may be one day. The entirety of the offset temperature data from the first temperature sensor 110a may be identified as irrelevant offset temperature data if the standard deviation for the offset temperature data from first temperature sensor 110a over the time period is 12.

In another example, the offset temperature difference may also be calculated for the offset temperature data for each temperature sensor 110 over the time period. The offset temperature difference may be calculated by taking the difference between the highest temperature value and the lowest temperature value in the series of offset temperature data (e.g. the first series of offset temperature data and/or the second series of offset temperature data) over the time period. The offset temperature difference may be compared to a temperature difference threshold. If the offset temperature difference calculation exceeds a temperature difference threshold, then the offset temperature data for the sensor may be identified as irrelevant offset temperature data and removed. The temperature difference threshold may, for example, be 15 degrees Celsius. The offset temperature difference may also be calculated over the time period or over a moving window. For the moving window, the offset temperature difference may be calculated over the time window ending at the current instant. The offset temperature data from the time window may be identified as irrelevant offset temperature data when the offset temperature difference calculation for the moving time window exceeds the temperature difference threshold.

In another example, the fluctuation of each temperature sensor is measured and compared to a fluctuation threshold to identify cases when a temperature sensor is partially broken. The fluctuations may be a difference calculation such as a range calculation or may be a difference from a mean value. The fluctuations in temperature may be measured from one temperature reading to the next temperature reading. Alternatively, the fluctuations may be measured by taking averages of small portions of temperature readings, for example, the average fluctuation of a minute of temperature readings. Measurement-to-measurement fluctuations may be calculated by taking the difference from two consecutive measurements. As each new temperature measurement is recorded, the measurement-to-measurement fluctuation may be updated. The fluctuation may be compared to a fluctuation threshold. When the fluctuation exceeds the fluctuation threshold, the offset temperature data from the temperature sensor is identified as irrelevant offset temperature data and removed.

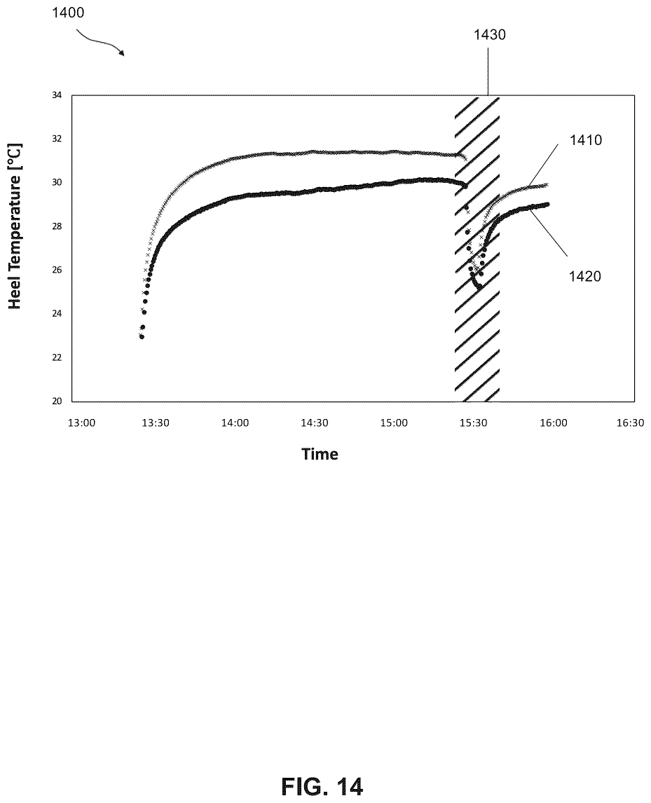

A second example of irrelevant offset temperature data may be identified using a loss of contact filter 1320. The loss of contact filter 1320 may include offset temperature data indicating loss of contact with the input device 100. The input device 100 may require close contact with the user's foot to accurately detect temperature, and a reduction in the integrity of the foot contact may result in inaccurate foot health change flags. The high variance may be determined using the same method as described for the partially broken sensor filter 1310, using a standard deviation calculation. The standard deviation may be calculated for the series of offset temperature data over the time period. The standard deviation may be compared to a standard deviation threshold and the offset temperature data for the temperature sensor may be identified as irrelevant offset temperature data if the standard deviation over a portion of the time period exceeds the standard deviation threshold. The portion of the series of offset temperature data occurring over the portion of the time period may be removed when the standard deviation over the portion of the time period exceeds the standard deviation threshold. In some examples, the standard deviation may be calculated over a moving time window within the time period. The moving window may be the duration of the portion of the time period. The moving window may include the time before the current instant where offset temperature data is being continuously generated. For example, the moving time window may be a 15-minute moving window, where the standard deviation is calculated at each instant and the time windows with a standard deviation exceeding the standard deviation threshold are identified as irrelevant offset temperature data and removed. The standard deviation for the partially broken sensor filter 1310 and the loss of contact filter 1320 may differ by the length of time used to calculate the variance. In some examples, a partially broken sensor may be detected when the standard deviation exceeds the standard deviation threshold over a longer time period (i.e. one day). A loss of contact may be detected when the standard deviation exceeds the standard deviation threshold over a short time period (e.g. 15 minutes). An example of the irrelevant offset temperature data determined using the loss of contact filter 1320 is shown in FIG. 14.

Another filter in the irrelevant offset temperature data module 1300 may include a completely broken sensor filter 1330, which may identify irrelevant offset temperature data from a completely broken sensor. A completely broken sensor may be caused, for example, by a short circuit or broken circuit connected to the temperature sensor. The temperature readings of a completely broken sensor may present with unusually high or low temperature values, which may be detected with a broken sensor low threshold and a broken sensor high threshold. The broken sensor thresholds may be defined using values that are abnormal for regular plantar temperatures or may be determined by testing the temperature readings from broken sensors caused by various issues (e.g. short circuit). A broken circuit may be detected by determining when the offset temperature data from a temperature sensor falls below a broken sensor low threshold (e.g. −10 degrees Celsius). Each of the offset temperature data values that fall below the broken sensor low threshold may be identified as irrelevant offset temperature data and removed. Alternatively, when a single offset temperature data value falls below the broken sensor low threshold, the entirety of the offset temperature data for the completely broken sensor may be removed. A short circuit may be detected by determining when the offset temperature data from a temperature sensor exceeds a broken sensor high threshold (e.g. 50 degrees Celsius). Each of the offset temperature data values that exceed the broken sensor high threshold may be identified as irrelevant offset temperature data and removed. Alternatively, when a single offset temperature data value exceeds the broken sensor high threshold, the entirety of the offset temperature data for the completely broken sensor may be removed.

An alert may also be initiated when the offset temperature data is identified as irrelevant offset temperature data using the partially broken sensor filter 1310 or completely broken sensor filter 1330. The recognition of a partially or completely broken sensor may be reported to the user, or third party using, for example, the output device and/or the display. A broken sensor may result in a portion of the user's foot not being monitored by the input device 100, which may increase the likelihood of the user developing a foot health change. The user or third party may be encouraged to increase visual inspection of the plantar areas under the broken sensors or may initiate the replacement of the input device 100.

Another filter in the irrelevant offset temperature data module 1300 may include a short data session filter 1340. The short data session filter 1340 may identify data sessions of a short length, including data sessions of offset temperature data where the input device 100 is donned, worn, and doffed. The data sessions may also include offset temperature data from the input device 100 being woken from sleep mode, but not worn by the user. For example, the user may wake the input device 100 by initiating motion, which starts the collection of temperature readings. However, the input device 100 may never be positioned on the user's foot and the resulting offset temperature data may cause inaccurate foot health change flags. There may also be one or more data sessions in the time period. The data sessions may be separated by periods of inactivity (i.e. the input device 100 is in sleep mode and no temperature readings are collected). The periods of inactivity may be, for example, at least 30 minutes long to be designated as separate data sessions. Short data sessions may not be long enough for the input device 100 to warm-up and equalize to the user's plantar temperature and may result in inaccurate foot health change flags. A data session collected when the input device 100 is not positioned on the foot may also result in a short data session. Any data sessions shorter than a data session threshold may be identified as irrelevant offset temperature data and removed. For example, the data session threshold may be 62 minutes because the temperature sensors 110 may take 50 minutes to warm up and 12 minutes to cool down. In the example, any session less than 62 minutes may be identified and removed as irrelevant offset temperature data.

Another filter in the irrelevant offset temperature data module 1300 may include a warm up and cool down filter 1350, which may identify the temperature offset temperature data from the warm up and cool down time for the temperature sensors 110. In a first case, when the input device 100 is donned by the user, the temperature sensors 110 may take time to equilibrized to the user's plantar temperature. The equilibrizing time for each temperature sensor may depend on the plantar location of the temperature sensor and/or the type of temperature sensor and input device 100. The heat transfer to more distal plantar locations (e.g. toes) may be slower than to proximal plantar locations. Also, a thicker input device 100 may take longer to equilibrize to plantar temperatures than a thinner input device 100. The warm up time may be captured by an initial wear time window, which occurs within the time period. The data collected in the initial wear time window may start when the user dons the input device (e.g. at the beginning of a data session). The initial wear time window may be identified as irrelevant offset temperature data and removed. For example, the temperature sensors 110 may take 30 minutes to warm-up to an equilibrized temperature. The first 30 minutes of each data session may be the initial wear time window and the data collected may be identified as irrelevant offset temperature data and removed.

The cool down of the input device 100 may occur when the user has doffed the input device 100. There may be two different cool down scenarios after the input device 100 is doffed by the user. The first scenario may be a natural cool down and the second scenario may be a charging cool down. In the first scenario, the user may allow the input device 100 to cool down to an external temperature (e.g. room temperature) after doffing the input device 100. The external temperature cool down period may be determined by reviewing the last portion of the data session (e.g. last 5 minutes). When the input device 100 is not connected to a charger, data session ends, and temperature readings cease to be collected when the input device 100 enters sleep mode (e.g. from lack of movement). The temperature readings collected during the last portion of the data session may be evaluated to identify the external temperature cool down period. For example, the average of the temperature readings from the left and right input device 100 may be taken over the last 5 minutes of a data session and compared to a cool down threshold. If the cool down threshold is satisfied, an end of wear time window may be used to remove the temperature readings from the cool down period. The end of wear time window may also occur within the time period and may end when the user doffs the input device 100. The end of wear time window may be identified as irrelevant offset temperature data and removed. For example, the external cool down period may be identified when the averages of the left and right input devices are lower than a cool down threshold of 25 degrees Celsius. The end of wear time window may include the offset temperature readings within, for example, the last 60 minutes of wear.

In another case, as shown in FIG. 2, the input device 100 may be powered by a charger, such as a wireless charger, which includes the wireless charging transmitter and wireless charging receiver 126. The wireless charging receiver 126 may be disposed within the input device 100. In the second scenario, the user may connect the wireless charging transmitter to the wireless charging receiver 126 directly after doffing the input device 100. The temperature sensors 110 may stop collecting temperature readings when wireless charging is occurring (i.e. temperature readings are not collected when the wireless charger is in use), resulting in the end of the data session. The cool down period may be short (i.e. shorter than the end of wear time window in the first scenario) to accommodate for the time taken to doff the input device 100 and place the wireless charging transmitter in proximity to the wireless charging receiver 126 to initiate wireless charging. In another example, the last 10-minutes of the data session may be filtered out when the data session ends in wireless charging.

The wireless charging process may cause excess heat near the wireless charging receiver 126, which may produce inaccurate foot health change flags when the input device 100 is donned directly after wireless charging. A wireless charging receiver heat filter 1360 may be used to remove irrelevant offset temperature data artificially inflated by wireless charging heat. The offset temperature data from temperature sensors 110 in proximity to the wireless charging receiver may be compared to a wireless charging temperature threshold. The offset temperature data derived from temperature sensors 110 positioned in proximity to the wireless charging receiver 126 may be identified as irrelevant offset temperature data and removed when the offset temperature data exceeds the wireless charging temperature threshold. The wireless charging temperature threshold may be calculated using offset temperature data derived from temperature sensors not in proximity to the wireless charging receiver 126. For example, the wireless charging temperature threshold may be calculated using the mean of the offset temperature data from temperature sensors 110 not in proximity to the wireless charging receiver 126. The proximity metric may be determined using a set distance value to the wireless charging receiver 126. For example, the proximity metric may be 5 cm and any temperature sensor touching the 5 cm radius from the edge of the wireless charging receiver 126 may be designated as “in proximity”. Any temperature sensor not touching the 5 cm radius may be designated as “not in proximity”.

In another example, the input device 100 may include five temperature sensors located under each one of the heel, the first metatarsal joint, the third metatarsal joint, the fifth metatarsal joint, and the hallux. The temperature sensor in the heel may be the only sensor in proximity to the wireless charging receiver 126. The wireless charging temperature threshold may be calculated using some combination of the offset temperature data from the temperature sensor under the first metatarsal joint, the third metatarsal joint, the fifth metatarsal joint, and the hallux (e.g. mean of the first metatarsal joint, the third metatarsal joint, the fifth metatarsal joint, and the hallux). The offset temperature data in the heel may be compared to the wireless charging temperature threshold and any offset temperature data from the heel that exceeds the wireless charging temperature threshold may be identified as irrelevant offset temperature data and removed.

Another example of irrelevant offset temperature data may include offset temperature data indicating the input device 100 was not worn by the user, which may be identified using an input device not worn filter 1370. Specifically, the irrelevant offset temperature data includes any data session where the input device 100 was not worn by the user. For example, the data session where the input device 100 was not worn may be caused by waking of the input device 100 without the input device 100 being donned by the user. Data sessions occurring when the input device 100 is worn by the user resulting in temperature readings that increase from an external temperature to the plantar temperatures of the user. When the input device 100 is not donned by the user, the temperature readings remain equilibrated at the external temperature (e.g. room temperature). The method for detecting a data session where the input device 100 was not worn by the user may include detecting when the temperature readings for the data session exceed an external temperature threshold. For example, the average of the offset temperature data for the left and right input devices 100 may be taken over the data session and compared to an external temperature threshold of 25 degrees Celsius. The offset temperature data from the data session may be identified as irrelevant offset temperature data if the averages remain below 25 degrees Celsius. In another example, each of the offset temperature data points are compared to the external temperature threshold. Any offset temperature data points falling below the external temperature threshold may be identified as irrelevant offset temperature data. Data sessions where the input device 100 is not worn may also be identified by the short data session filter 1340. If the data session has already been removed by the short data session filter 1340, then no further offset temperature data removal is required.

In examples where portions or the entirety of offset temperature data from a certain temperature sensor are removed, the resulting temperature difference calculations (step 350 in FIG. 3) for those sensors may be adjusted. The temperature difference may not be calculated when a temperature reading for a time point required for the calculation has been removed, or replaced with an error or NaN value. Alternatively, the temperature difference calculation may result in an error or NaN value, itself. The error or NaN value may not be used to determine the presence of a foot health change. In examples where the temperature difference is calculated with an average of multiple temperature sensors 110, the average may be calculated without the processed temperature data from the temperature sensor with an error value, NaN value, or missing value. In other examples, the average may be calculated using the remaining processed temperature data with numerical values.

Another example of irrelevant offset temperature data may include high activity temperature readings, which may be determined using an activity classification algorithm or predictive algorithm for the user's daily patterns. An example of an activity classification algorithm that may be used to classify sensor data is described in U.S. Pat. No. 11,526,749 entitled “METHOD AND SYSTEM FOR ACTIVITY CLASSIFICATION”, the entirety of which is incorporated herein by reference. The temperature offset may be determined by recognizing various user activities and adjusting the temperature offset based on the type of activity. The activities that may be identified using the activity classification algorithm include donning the input device 100, doffing the input device 100, and high intensity activities (e.g. walking, running, jumping, etc.). The donning, doffing, and high intensity activities may be identified within the offset temperature data and removed. Similarly, the irrelevant offset temperature data may also be identified using a predictive model that can predict the daily circadian rhythm based on previous temperature readings and offset temperature data. The irrelevant offset temperature data may be determined based on the circadian rhythm of the user or deviations from the user's normal circadian rhythm.

Referring now to FIG. 14, shown therein is an example embodiment of the application of the loss of contact filter 1320 described in FIG. 13. FIG. 14 shows the first series of offset temperature data 1410 from the left heel temperature sensor and the second series of offset temperature data 1420 from the right heel temperature sensor, after the temperature offset has been applied. At approximately 15:30, the first series of offset temperature data 1410 and the second series of offset temperature data 1420 show high variance over a 15-minute portion of the time period, which may indicate a loss of contact between the input device 100 and the user's foot. The data points contained within the loss of contact filter box 1430 are the offset temperature data points identified as irrelevant offset temperature data and removed using the loss of contact filter 1320. The vertical axis shows the temperature values, and the horizontal axis shows the time. In the example, the temperature is in degrees Celsius, and the time is the local time in the time zone the input device and the user are located.

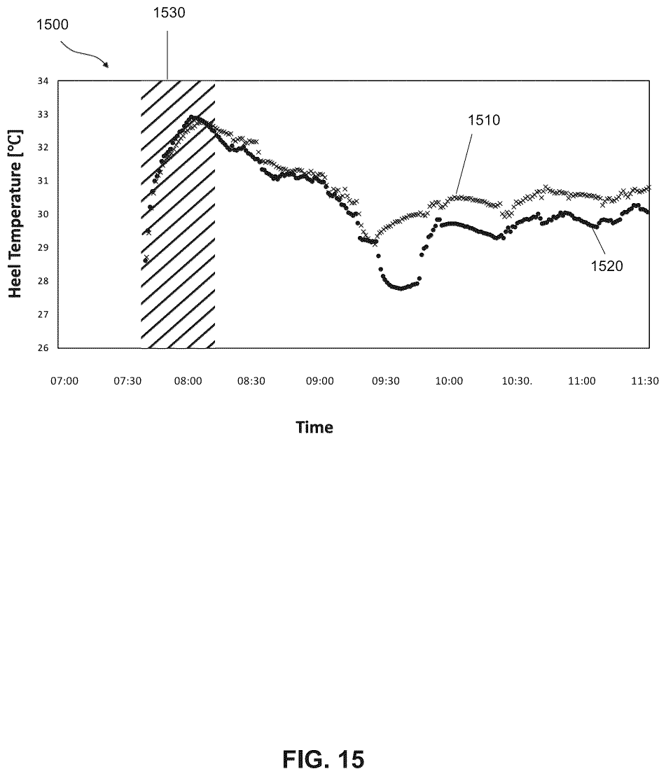

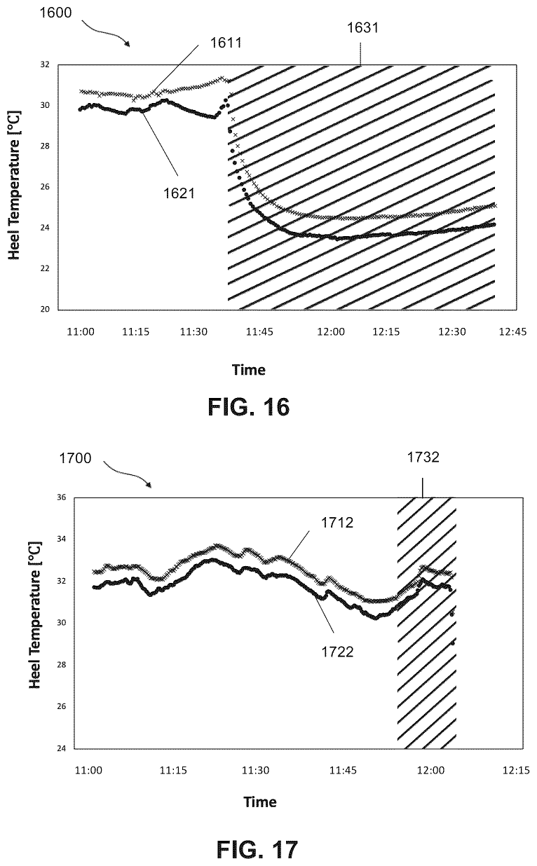

Referring now to FIG. 15, FIG. 16, and FIG. 17, shown therein is an example embodiment of the application of the warm up and cool down filter 1350 described in FIG. 13. FIG. 15 shows the first series of offset temperature data 1510 from the left heel temperature sensor and the second series of offset temperature data 1520 from the right heel temperature sensor, after the temperature offset is applied. The time period starts when the first offset temperature data point occurs, which may also be when the user dons the input device 100. At approximately 7:40, the warm up period begins after the input device 100 is donned. The offset temperature data points contained within the 30-minute warm up filter box 1530 are the offset temperature data points identified as irrelevant offset temperature data and removed using the warm up and cool down filter 1350. The vertical axis shows the temperature values, and the horizontal axis shows the time. In the example, the temperature is in degrees Celsius, and the time is the local time in the time zone the input device 100 and the user are located.

FIG. 16 and FIG. 17 show two potential cool down scenarios. FIG. 16 shows a first cool down scenario where the insole is not wirelessly charged after the input device 100 is doffed. The natural cool down temperature graph 1600 shows the temperature readings when the input device 100 is allowed to cool to an external temperature after being doffed by the user. The lack of wireless charging may be detected using the cool down threshold described in FIG. 13, above. As with the earlier examples, shown therein are the first series of offset temperature data 1611 from the left heel temperature sensor and the second series of offset temperature data 1621 from the right heel temperature sensor, after the temperature offset has been applied. The end of wear time window occurs when there is no wireless charging, which may be one hour in duration. At approximately 12:40, the cool down period ends when the input device 100 enters sleep mode after cooling to an external temperature. The cool down filter box 1631 contains the offset temperature data points between the 11:40 to 12:40 time points. The offset temperature data points contained within the one hour cool down filter box 1631 are the offset temperature data points identified as irrelevant offset temperature data and removed using the warm up and cool down filter 1350. The vertical and horizontal axes are the same as the above FIG. 15.

FIG. 17 shows a second cool down scenario where the insole is wirelessly charged after being doffed, instead of cooling down to external temperature. The charger cool down temperature graph 1700 shows the temperature readings when the input device 100 is wirelessly charged directly after doffing the input device 100. The wireless charging scenario may be detected using the cool down threshold described in FIG. 13. As with the earlier examples, shown therein are the first series of offset temperature data 1712 from the left heel temperature sensor and the second series of offset temperature data 1722 from the right heel temperature sensor, after the temperature offset has been applied. The end of wear time window ends when wireless charging is initiated, and the temperature readings are no longer collected. The end of wear time window without charging may be 10-minutes in duration. At approximately 12:05, the cool down period ends when the input device 100 is placed on the wireless charger and the temperature data collection ends. The cool down filter box 1732 contains the offset temperature data points between the 11:55 to 12:05 time points. The offset temperature data points contained within the 10-minute cool down filter box 1732 are the offset temperature data points identified as irrelevant offset temperature data and removed using the warm up and cool down filter 1350. The vertical and horizontal axes are the same as the above FIG. 15.

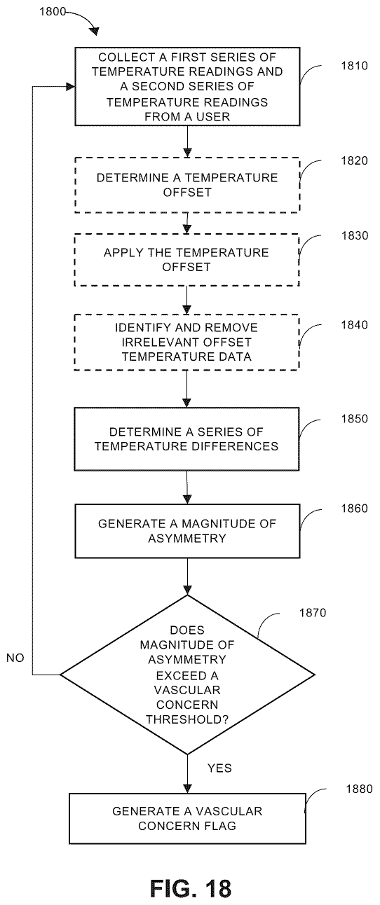

Referring now to FIG. 18, shown therein is example method 1800, which applies a similar method to method 300 of FIG. 3. Method 1800 is directed towards generating a vascular concern flag when circulatory or vascular flow issues from any part of the body that can be detectable by a wearable temperature sensor or a temperature sensor positionable on the foot are detected (e.g. blood flow issues such as a blocked or partially blocked artery in the leg may manifest as a temperature difference in the foot). The vascular concern flag is a foot health change flag. The method 1800 can also be used to evaluate the effectiveness of an intervention for vascular flow issues. The vascular concern flag is dependent on detecting an asymmetrical foot temperature pattern. The asymmetrical foot temperature pattern may indicate an asymmetrical vascular disease foot health change. The asymmetrical foot temperature pattern foot health change is a temperature difference between two plantar locations of the user caused by, for example, microvascular disease, macrovascular disease, or both in a foot, lower limb, or at another location of the body of the user.

At step 1810, temperature readings are collected from the user. The temperature readings include a first series of temperature readings and a second series of temperature readings. The temperature readings may be collected from the temperature sensors 110 of input device 100. At each of a series of time points over the time period, a first temperature reading is collected at a first plantar location on the body of the user, to yield a first series of temperature readings. Additionally, at each of a series of time points over the time period, a second temperature reading is collected at a second plantar location on the body of the user, to yield a second series of temperature readings. The first plantar location and the second plantar location may be located on the same foot of the user (i.e. ipsilateral plantar locations). In one example, the two plantar locations of the user may include a first plantar location relating to a first angiosome of one foot of the user and a second ipsilateral plantar location relating to a second angiosome of the same foot of the user. Alternatively, the two plantar locations may be located contralaterally. In a specific example, the first plantar location is on the left foot and is contralateral to the second location on the right foot of the user's feet. A time period for temperature collection from the user can have any length and the first and second series of temperature readings from a user may be collected over one or multiple time periods. For evaluating trends in temperature differences over time or for evaluating the effectiveness of a vascular intervention, the collection of temperature readings from the user may occur over one or multiple time periods such as over a span of minutes to over 10 years.

At optional step 1820, a temperature offset is determined. Similar to step 320 of method 300, the temperature offset may be used to adjust the temperature readings to increase accuracy of the vascular concern flag. The temperature offset may be determined using historical wear session temperature readings from the user or historical temperature readings from the input device 100. The method for determining the temperature offset may vary depending on the type of temperature offset being applied. Examples of temperature offsets and methods of determining temperature offsets are described in FIG. 10.

At optional step 1830, the temperature offset is applied to the first and second series of temperature readings. The temperature offset may be applied to the first series of temperature readings to generate a first series of offset temperature data. Additionally, the temperature offset may be applied to the second series of temperature readings to generate a second series of offset temperature data. The temperature offset may be a single temperature offset, such as a single temperature offset applied to one or more series of temperature readings. The temperature offset may also include multiple temperature offsets applied together to one or more series of temperature readings. The temperature offset may further include a unique temperature offset applied to each series of temperature readings, separately, or the temperature offset may include the same temperature offset applied to each series of temperature readings.

At optional step 1840, irrelevant offset temperature data is identified and removed from the first and second series of offset temperature data or by removing the corresponding temperature readings from the first and second series of temperature readings. The irrelevant offset temperature data may be identified using one or more thresholds. Examples of the filters that may be used to identify irrelevant offset temperature data are described in FIG. 13. A first series of processed temperature data is generated once the irrelevant offset temperature data is removed from the first series of temperature readings or from the first series of offset temperature data. Similarly, a second series of processed temperature data is generated once the irrelevant offset temperature data is removed from the second series of temperature readings or from the second series of offset temperature data. The irrelevant offset temperature data may be identified and removed from the first series of offset temperature data and from the second series of offset temperature data, individually. Alternatively, the same irrelevant offset temperature data may be identified in one series of offset temperature data and applied to each series of offset temperature data. The irrelevant offset temperature data may be removed by replacing the offset temperature data points with an NaN value or missing value and ignored or may be replaced with a synthesized or estimated real temperature difference value using interpolation or some other method. The irrelevant offset temperature data are not used in any further steps. Additional data preprocessing steps may be taken.

At step 1850, a series of temperature differences is determined. A series of temperature differences may be the result of the difference between two temperature series, or the result of the difference of at least a first temperature value from a first temperature series and at least a second temperature value from a second temperature series. If optional steps 1820, 1830 and 1840 were applied, a series of temperature differences is determined by comparing the first series of processed temperature data and the second series of processed temperature data for each time point in the time period. If optional steps 1820, 1830 and 1840 were not applied, the series of temperature difference is determined by comparing the first series of temperature readings and the second series of temperature readings for each time point in the time period. Similar to step 350 of method 300, the temperature difference may be a contralateral temperature difference or an ipsilateral temperature difference, or some combination thereof. A contralateral temperature difference may be calculated between readings taken from a first plantar location on a first foot and a second plantar location on a second foot. The contralateral temperature differences may be applied to processed temperature data derived from temperature readings collected by temperature sensors under equivalent plantar structures of the opposite feet. An ipsilateral temperature difference may be calculated between a first temperature sensor and second temperature sensor on the same foot of the user. When determining the ipsilateral temperature difference calculation, the second series of temperature readings may include calculating the average of some or all of the first and second series of processed temperature data, offset temperature data, or temperature readings. Alternatively, the ipsilateral temperature difference may include calculating the difference between the first series of processed temperature data and the average of the first and second series of processed temperature data or temperature readings. In one example, the first series of processed temperature data or temperature readings may be collected by a temperature sensor at a first plantar location related to a first angiosome of the foot and the second series of processed temperature data or temperature readings may be collected by a temperature sensor at a second ipsilateral plantar location related to a second angiosome of the foot. In other examples, the input device 100 includes a temperature sensor array 108 with more than two temperature sensors. Temperature readings may be taken at a plurality of plantar locations on the user's first foot and the second temperature reading at the second plantar location may include a series of average temperature readings, which is determined by calculating an average of the plurality of temperature readings collected by the input device 100 at each time point over the time period. In another example, temperature differences may be determined between a first series of average temperature readings taken from temperature sensors at a first plantar location related to a first angiosome of the foot and a second series of average temperature reading taken from temperature sensors at a second ipsilateral plantar location related to a second angiosome of the foot, each series of average temperature readings being determined by calculating an average of the plurality of temperature readings collected by the input device 100 at each time point over the time period.

At step 1860, a magnitude of asymmetry is determined by calculating a measure of central tendency of the series of temperature differences. The measure of central tendency may be an average, a median, a 95% confidence interval of the series of temperature differences, a percentage threshold, or other measure of central tendency.

At step 1870, the magnitude of asymmetry is compared to a vascular concern threshold. The vascular concern threshold is a foot health change threshold. The magnitude of asymmetry may be converted to absolute values, or the temperature differences may be reversed to produce positive values. The vascular concern threshold may be a single value (e.g. 1.0 degree Celsius) and the magnitude of asymmetry may be compared to the single threshold value.

If the magnitude of asymmetry exceeds the vascular concern threshold, the method proceeds to step 1880. If the average temperature difference does not exceed the vascular concern threshold, method 1800 does not proceed any further, and is instead repeated from step 1810. The user may not have the need for a vascular procedure or other intervention when the magnitude of asymmetry does not exceed the vascular concern threshold, however, the method may optionally prompt the user or a third party for an assessment (e.g. physical exam or further investigations). The vascular concern threshold may be related to an accepted value indicating early warning signs of a vascular health change. In one example, the vascular concern threshold may be related to the improvement, reversal, or recidivism of a health change such as revascularization affecting blood flow after medication administration, a vascular procedure, or other intervention. If the magnitude of asymmetry falls below the vascular concern threshold, the success of the vascular procedure or intervention administered may be confirmed.

At step 1880, a vascular concern flag is generated. The vascular concern flag may include an alert. The vascular concern flag may be presented to the user and/or to a third party. A vascular concern flag may occur in various areas of the foot and the foot flag may be generated where the concern is identified. The location of the vascular concern on the user's foot may be identified as a flag on a digital model of a foot, lower limb or other body part. The digital model of the foot may be reviewed by the user and/or a third party. The magnitude of asymmetry and the location of the vascular concern flag may be indicated on a thermal image display of the foot, lower limb or other body part, on a digital dashboard. In other examples, the vascular concern flag may be presented as a general alert to the user, without an indication of the location on the foot.

In addition to generating the vascular concern flag, the flag for vascular concern may be indicated to a third party, and the third party may initiate a personalized temperature offset to prevent any further alerting. Alternatively, the personalized temperature offset may be applied as part of step 330 in method 300, step 430 in method 400, step 530 in method 500, step 1830 in method 1800, or step 1930 in method 1900. A personalized temperature filter may also be calculated to remove irrelevant temperature readings caused by the vascular concern. The input device 100 may then be used to detect worsening of the vascular concern and/or additional foot health changes. The third party may initiate a care action after the vascular concern flag is generated. In other examples, the care action may include an intervention such as an input device 100 modification, amputation, an assessment, imaging, diagnostic tests, physical exams, prescription of an assistive device, a vascular procedure, medication, pharmacological therapy, activity/movement prescription, or the cessation or reversal of a care action. In one example, the input device 100 continues monitoring at least one of posture, activity, or movement of the user by collecting pressure or rate of change of movement measurements at a location on the body of the user. In another example, pressure measurements include at least one pressure sensor or at least one accelerometer disposed in footwear, an insole, a pedometer, a watch, a wearable, a smartphone, or another tracker worn by user.

After step 1880, the method 1800 may proceed again from step 1810 to continue monitoring of the user's vascular asymmetry status. In other examples, after step 1880, the method 1800 is followed by methods 300, 400, 500, 600, 700, 800, 900, or 1900. The continuous monitoring of the user aids in tuning out noise generated by the input system 100 over time.

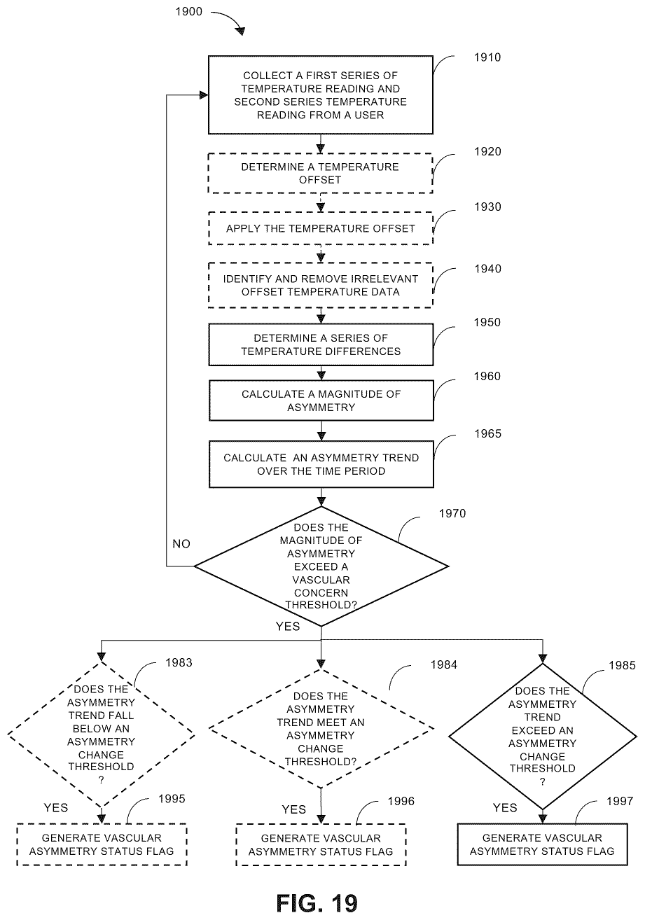

Referring now to FIG. 19, shown therein is an example embodiment of the application of generating a vascular asymmetry status flag, which applies a similar method to method 300 of FIG. 3, and method 1800 of FIG. 18. Method 1900 is directed towards temperature readings for detecting if a vascular asymmetry status is improving, staying the same, or degrading over time. The vascular asymmetry status flag generated can be caused by circulatory or vascular flow issues in the foot of the user, or from flow issues from any part of the body that can be detectable by a wearable temperature sensor or a temperature sensor positionable on the foot (e.g. blood flow issues such as a blocked or partially blocked artery in the leg may manifest as a temperature difference in the foot). The vascular asymmetry status flag is a foot health change flag. The method 1900 can also be used to evaluate the effectiveness of an intervention for vascular flow issues. The vascular asymmetry flag is dependent on detecting an asymmetrical foot temperature pattern. The asymmetrical foot temperature pattern may indicate an asymmetrical vascular disease foot health change. The asymmetrical foot temperature pattern foot health change is a temperature difference between two plantar locations of the user caused by, for example, microvascular disease, macrovascular disease, or both in a foot, lower limb, or at another location of the body of the user.

At step 1910, temperature readings are collected from the user. The temperature readings include a first series of temperature readings and a second series of temperature readings. The temperature readings may be collected from the temperature sensors 110 of input device 100. At each of a series of time points over the time period, a first temperature reading is collected at a first plantar location on the body of the user, to yield a first series of temperature readings. Additionally, at each of a series of time points over the time period, a second temperature reading is collected at a second plantar location on the body of the user, to yield a second series of temperature readings. The first plantar location and the second plantar location may be located on the same foot of the user (i.e. ipsilateral plantar locations). In one example, the two plantar locations of the user may include a first plantar location relating to a first angiosome of one foot of the user and a second ipsilateral plantar location relating to a second angiosome of the same foot of the user. Alternatively, the two plantar locations may be located contralaterally. In a specific example, the first plantar location is on the left foot and is contralateral to the second location on the right foot of the user's feet. A time period for temperature collection from the user can have any length and the first and second series of temperature readings from a user may be collected over one or multiple time periods. For evaluating trends in temperature differences over time or for evaluating the effectiveness of a vascular intervention, the collection of temperature readings from the user may occur over one or multiple time periods such as over a span of minutes to over ten years.

At optional step 1920, a temperature offset is determined. Similar to step 320 of method 300, and step 1820 of method 1800, the temperature offset may be used to adjust the temperature readings to increase accuracy of the vascular asymmetry status flag. The temperature offset may be determined using historical wear session temperature readings from the user or historical temperature readings from the input device 100. The method for determining the temperature offset may vary depending on the type of temperature offset being applied. Examples of temperature offsets and methods of determining temperature offsets are described in FIG. 10.

At optional step 1930, the temperature offset is applied to the first and second series of temperature readings. The temperature offset may be applied to the first series of temperature readings to generate a first series of offset temperature data. Additionally, the temperature offset may be applied to the second series of temperature readings to generate a second series of offset temperature data. The temperature offset may be a single temperature offset, such as a single temperature offset applied to one or more series of temperature readings. The temperature offset may also include multiple temperature offsets applied together to one or more series of temperature readings. The temperature offset may further include a unique temperature offset applied to each series of temperature readings, separately, or the temperature offset may include the same temperature offset applied to each series of temperature readings.

At optional step 1940, irrelevant offset temperature data is identified and removed from the first and second series of offset temperature data or by removing the corresponding temperature readings from the first and second series of temperature readings. The irrelevant offset temperature data may be identified using one or more thresholds. Examples of the filters that may be used to identify irrelevant offset temperature data are described in FIG. 13. A first series of processed temperature data is generated once the irrelevant offset temperature data is removed from the first series of temperature readings or from the first series of offset temperature data. Similarly, a second series of processed temperature data is generated once the irrelevant offset temperature data is removed from the second series of temperature readings or from the second series of offset temperature data. The irrelevant offset temperature data may be identified and removed from the first series of offset temperature data and from the second series of offset temperature data, individually. Alternatively, the same irrelevant offset temperature data may be identified in one series of offset temperature data and applied to each series of offset temperature data. The irrelevant offset temperature data may be removed by replacing the offset temperature data points with an NaN value or missing value and ignored or may be replaced with a synthesized or estimated real temperature difference value using interpolation or some other method. The irrelevant offset temperature data are not used in any further steps. Additional data preprocessing steps may be taken.

At step 1950, a series of temperature differences is determined. A series of temperature differences may be the result of the difference between two temperature series, or the result of the difference of at least a first temperature value from a first temperature series and at least a second temperature value from a second temperature series. If optional steps 1920, 1930 and 1940 were applied, a series of temperature differences is determined by comparing the first series of processed temperature data and the second series of processed temperature data for each time point in the time period. If optional steps 1920, 1930 and 1940 were not applied, the series of temperature difference is determined by comparing the first series of temperature readings and the second series of temperature readings for each time point in the time period. Similar to step 350 of method 300, and step 1850 or method 1800, the temperature difference may be a contralateral temperature difference or an ipsilateral temperature difference, or some combination thereof. A contralateral temperature difference may be calculated between readings taken from a first plantar location on a first foot and a second plantar location on a second foot. The contralateral temperature differences may be applied to processed temperature data derived from temperature readings collected by temperature sensors under equivalent plantar structures of the opposite feet. An ipsilateral temperature difference may be calculated between a first temperature sensor and second temperature sensor on the same foot of the user. When determining the ipsilateral temperature difference calculation, the second series of temperature readings may include calculating the average of some or all of the first and second series of processed temperature data, offset temperature data, or temperature readings. Alternatively, the ipsilateral temperature difference may include calculating the difference between the first series of processed temperature data and the average of the first and second series of processed temperature data or temperature readings. In one example, the first series of processed temperature data or temperature readings may be collected by a temperature sensor at a first plantar location related to a first angiosome of the foot and the second series of processed temperature data or temperature readings may be collected by a temperature sensor at a second ipsilateral plantar location related to a second angiosome of the foot. In other examples, the input device 100 includes a temperature sensor array 108 with more than two temperature sensors. Temperature readings may be taken at a plurality of plantar locations on the user's first foot and the second temperature reading at the second plantar location may include a series of average temperature readings, which is determined by calculating an average of the plurality of temperature readings collected by the input device 100 at each time point over the time period. In another example, temperature differences may be determined between a first series of average temperature readings taken from temperature sensors at a first plantar location related to a first angiosome of the foot and a second series of average temperature reading taken from temperature sensors at a second ipsilateral plantar location related to a second angiosome of the foot, each series of average temperature readings being determined by calculating an average of the plurality of temperature readings collected by the input device 100 at each time point over the time period.

At step 1960, a magnitude of asymmetry is determined by calculating a measure of central tendency of the series of temperature differences. The measure of central tendency may be an average, a median, a 95% confidence interval of the series of temperature differences, a percentage threshold, or other measure of central tendency.

At step 1965, an asymmetry trend is determined by calculating a measure of asymmetry trending from the series of temperature differences over the time period. An asymmetry trend may include a rate of change of the series of temperature differences. In an example, a weighted average may be used, where temperature readings are weighted by usage duration, step count, or some other measure of user behavior. In another example, the asymmetry trend may be established by determining a modelled linear or non-linear fit of a set of historical data. The asymmetry trend may be calculated by removing outliers or irrelevant data points, cleaning the data, or transforming the temperature data prior to the asymmetry trend calculation. A correlation or other measure of association may be computed to quantify the strength of the asymmetry trend calculation. The asymmetry trend may be increasing, decreasing or staying the same over the time period. The asymmetry trend over the time period may be determined (i.e. step 1965) any time after step 1950 and before steps 1983, 1984 or 1985.

At step 1970, the magnitude of asymmetry is compared to a vascular concern threshold. The vascular concern threshold is a foot health change threshold. The magnitude of asymmetry may be converted to absolute values, or the temperature differences may be reversed to produce positive values. The vascular concern threshold may be a single value (e.g. 1.0 degree Celsius) and the magnitude of asymmetry may be compared to the single threshold value.

If the magnitude of asymmetry exceeds the vascular concern threshold, the method proceeds to steps 1983, 1984, or 1985. In some examples, the user may no longer have a vascular asymmetry, or the magnitude of their vascular asymmetry may not exceed the vascular concern threshold, however, the method may optionally prompt the user or a third party for an assessment (e.g. physical exam or further investigations). When the magnitude of asymmetry does not exceed the vascular concern threshold, method 1900 does not proceed any further, and is instead repeated from step 1910 (e.g. the user vascular status continues to be monitored for any changes). The vascular concern threshold may be related to an accepted value indicating a vascular health change (for example but not limited to the following, the vascular concern threshold may be a 1.0 degree Celsius temperature difference). When the magnitude of asymmetry exceeds the vascular concern threshold (i.e. the method determines vascular asymmetry is present), the method proceeds to compare the asymmetry trend to an asymmetry change threshold. The asymmetry change threshold may be related to an accepted vascular health change rate. If the asymmetry trend falls below the asymmetry change threshold, the vascular status may be improving (i.e. the vascular asymmetry is getting better over time) and if an intervention has been applied, the success of the vascular procedure or intervention administered may be deemed to be effective and the method proceeds to step 1995. If the asymmetry trend meets the asymmetry change threshold, there may be no vascular status improvement over the time period and if an intervention has been applied, the success of the vascular procedure or intervention administered may not be confirmed and the method proceeds to step 1996 (i.e. the success of the intervention administered may not be confirmed or it may have no impact). If the asymmetry trend falls above the asymmetry change threshold, the vascular status may be degrading (i.e. the vascular asymmetry is getting worse over time) and if an intervention has been applied, the success of the vascular procedure or intervention administered may be deemed to have no impact or not to be effective and the method proceeds to step 1997 (i.e. the success of the vascular procedure or intervention administered may not be confirmed).

At steps 1995, 1996, or 1997, a vascular asymmetry status flag is generated. The vascular asymmetry status flag may include an alert. The vascular asymmetry status flag may be presented to the user and/or to a third party. A vascular asymmetry status flag may occur in various areas of the foot and the flag may be generated where the vascular asymmetry status is identified. The location of the vascular asymmetry on the user's foot may be identified as a flag on a digital model of a foot, lower limb or other body part. The digital model of the foot may be reviewed by the user and/or a third party. The magnitude of asymmetry, asymmetry trend, and the location of the vascular asymmetry status flag may be indicated on a thermal image display of the foot, lower limb or other body part, on a digital dashboard. In other examples, the vascular asymmetry status flag may be presented as a general alert to the user, without an indication of the location on the foot. The vascular asymmetry status flag may be used to adjust the care actions or care level of the user. In some examples, the care action may include an intervention such as an input device 100 modification, amputation, an assessment, imaging, diagnostic tests, physical exams, prescription of an assistive device, a vascular procedure, medication, pharmacological therapy, activity/movement prescription, or the cessation or reversal of a care action.

In addition to generating the vascular asymmetry status flag, the flag for vascular asymmetry status may be indicated to a third party, and the third party may initiate a personalized temperature offset to prevent any further alerting. Alternatively, the personalized temperature offset may be applied as part of step 330 in method 300, step 430 in method 400, step 530 in method 500, step 1830 in method 1800, or step 1930 in method 1900. A personalized temperature filter may also be calculated to remove irrelevant temperature readings caused by the vascular asymmetry status. The input device 100 may then be used to detect worsening of the vascular asymmetry status and/or additional foot health changes. The third party may initiate a care action after the vascular asymmetry status flag is generated. In other examples, the care action may include an intervention such as an input device 100 modification, amputation, an assessment, imaging, diagnostic tests, physical exams, prescription of an assistive device, a vascular procedure, medication, pharmacological therapy, activity/movement prescription, or the cessation or reversal of a care action. In one example, the input device 100 continues monitoring at least one of posture, activity, or movement of the user by collecting pressure or rate of change of movement measurements at a location on the body of the user. In another example, pressure measurements include at least one pressure sensor or at least one accelerometer disposed in footwear, an insole, a pedometer, a watch, a wearable, a smartphone, or another tracker worn by user.

After steps 1995, 1996 or 1997, the method 1900 may proceed again from step 1910 to continue monitoring of the user's vascular asymmetry status. In other examples, after steps 1995, 1996 or 1997, the method 1900 is followed by methods 300, 400, 500, 600, 700, 800, 900, or 1800. The continuous monitoring of the user aids in tuning out noise generated by the input system 100 over time.

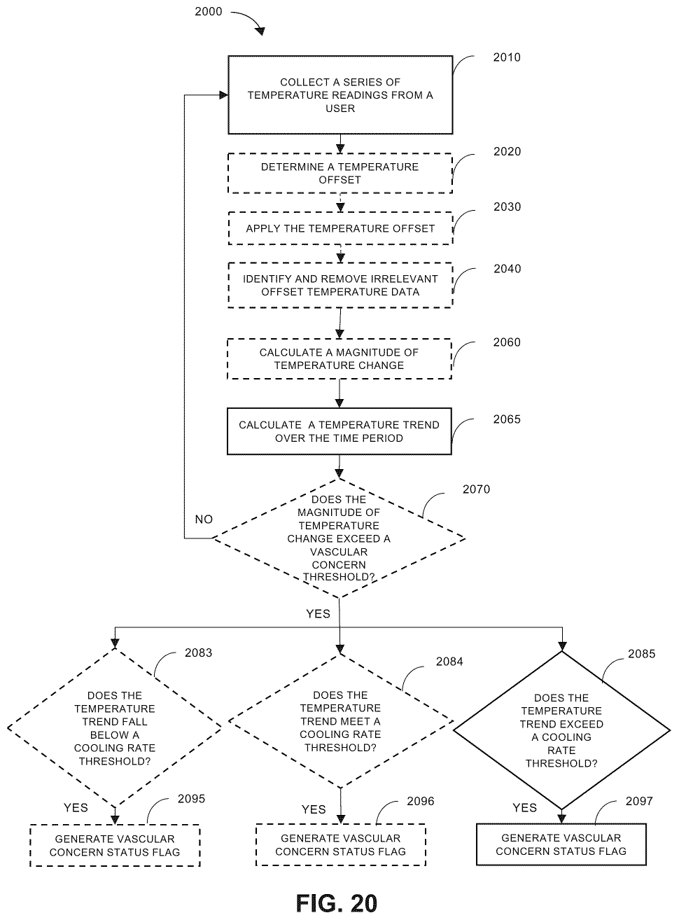

Referring now to FIG. 20, shown therein is an example embodiment of the application of generating a vascular concern status flag, which applies a similar method to method 300 of FIG. 3, and method 1900 of FIG. 19. Method 2000 is directed towards temperature readings for detecting if a vascular concern status is degrading or staying the same over time. The vascular concern status flag generated can be caused by circulatory or vascular flow issues in the foot of the user (e.g. peripheral vascular health), or from flow issues from any part of the body that can be detectable by a wearable temperature sensor or a temperature sensor positionable on the foot (e.g. blood flow issues such as a blocked or partially blocked artery in the leg may manifest as a temperature trend in the foot). In addition, the vascular concern status flag generated can be caused by inflammation (e.g. from a wound, an infection, etc.), seasonal fluctuations, environmental changes, user behaviors, or a combination of these factors. The vascular concern status flag is a foot health change flag. The method 2000 can also be used to evaluate the effectiveness of an intervention for vascular flow or inflammation issues, or the recidivism of disease. The vascular concern status flag is dependent on detecting a temperature trend of the temperature readings over time. The vascular concern flag may indicate a foot or lower limb health change. The vascular concern status flag corresponds to a temperature trend in the temperature readings of at least one plantar location of the user. In the case of a cooling temperature trend, the vascular concern status may be a qualitative or quantitative flag which may be indicative of vascular issues in the body of the user. In the case of a warming temperature trend of the temperature readings, the vascular concern status flag may be a qualitative or quantitative flag which may be indicative of vascular issues or inflammation in the body of the user. Method 2000 allows for the tracking of vascular issues when it may not be possible to determine a temperature difference between two temperature sensors 110 in the input device 100 or when only temperature readings from one temperature sensor can be collected. In one example, method 2000 tracks unilateral temperature measurements (i.e., tracking one temperature sensor under the portion remaining after amputation of a user's first foot and where a user has no second foot).

At step 2010, temperature readings are collected from the user. The temperature readings include a series of temperatures collected from the temperature sensor 110 of input device 100. At each time point over the time period, a temperature reading is collected at a plantar location on the body of the user, to yield a series of temperature readings. A time period for temperature collection from the user can have any length and the series of temperature readings from a user may be collected over one or multiple time periods, including one or more data sessions. For evaluating trends in temperature over time or for evaluating the effectiveness of a vascular intervention, the collection of temperature readings from the user may occur over one or multiple time periods such as over a span of minutes to over ten years. In one example, temperature readings may be taken from only one temperature sensor 110. In another example, temperature readings may be taken from a plurality of plantar locations on the user's foot or feet, or from locations relating to an angiosome relating to the user's foot. The temperature readings may be averaged to form a single series of averaged temperature readings (e.g. an average of the plurality or a subset of temperature readings collected by the input device 100 at each time point over the time period).

At optional step 2020, a temperature offset is determined. Similar to step 320 of method 300, and step 1920 of method 1900, the temperature offset may be used to adjust the temperature readings to increase accuracy of the vascular concern status flag. The temperature offset may be determined using historical wear session temperature readings from the user or historical temperature readings from the input device 100. The method for determining the temperature offset may vary depending on the type of temperature offset being applied. Examples of temperature offsets and methods of determining temperature offsets are described in FIG. 10.

At optional step 2030, the temperature offset is applied to the series of temperature readings to generate a series of offset temperature data. The temperature offset may be a single temperature offset or may also include multiple temperature offsets applied together to the series of temperature readings. The temperature offset may further include a unique temperature offset applied to the series of temperature readings. At optional step 2040, irrelevant offset temperature data is identified and removed from the series of offset temperature data or by removing the corresponding temperature readings from the series of temperature readings. The irrelevant offset temperature data may be identified using one or more thresholds. Examples of the filters that may be used to identify irrelevant offset temperature data are described in FIG. 13. A series of processed temperature data is generated once the irrelevant offset temperature data is removed from the series of temperature readings or from the series of offset temperature data. The irrelevant offset temperature data may be removed by replacing the offset temperature data points with an NaN value or missing value and ignored or may be replaced with a synthesized or estimated real temperature difference value using interpolation or some other method. The irrelevant offset temperature data are not used in any further steps. Additional data preprocessing steps may be taken.

At optional step 2060, a magnitude of temperature change is determined by calculating a difference of a first temperature reading and a second temperature reading from the series of temperature readings, by calculating the difference of the lowest and highest temperature, by calculating the difference of the outer values of a 95% confidence interval of the temperature readings, or other measure of magnitude from the series of temperature readings.

At step 2065, a temperature trend is determined by calculating a measure of temperature trend from the series of temperature readings over the time period. A temperature trend may include determining a slope or rate of change of the series of temperature readings. In an example, a weighted average may be used, where temperature readings are weighted by usage duration, step count, or some other measure of user behavior. In another example, the temperature trend may be established by determining a modelled linear or non-linear fit of a set of historical data. The temperature trend may be calculated by removing outliers or irrelevant data points, cleaning or preprocessing the data, or transforming the temperature data prior to the temperature trend calculation. A correlation or other measure of association may be computed to quantify the strength of the temperature trend calculation. The temperature trend may be determined to be increasing, decreasing or staying the same over the time period. The temperature trend may be utilized to evaluate if a vascular concern or improvement is positively or negatively impacting a site located a distance from the vascular concern (e.g. the temperature trend may be utilized to determine that a blockage in the left limb was causing a warming temperature trend in the right limb and after a subsequent re-vascularization procedure the temperature trend may be determined to show warming in the left limb and cooling in the right limb). The temperature trend over the time period may be determined (i.e. step 2065) any time after step 2010 and before steps 2083, 2084 or 2085. Optionally, the method 2000 continues only if the temperature trend is determined to be statistically significant.

At optional step 2070, the magnitude of temperature change is compared to a vascular concern threshold. The vascular concern threshold is a foot health change threshold. The magnitude of asymmetry may be converted to absolute values, or the temperature differences may be reversed to produce positive values. The vascular concern threshold may be a single value (e.g. 0.01 degree Celsius) and the magnitude of temperature change may be compared to the single threshold value. If the magnitude of temperature change exceeds the vascular concern threshold, the method may proceed to steps 2083, 2084, or 2085. In some examples, the user may no longer have a vascular or inflammation concern or the magnitude of the temperature change may not exceed the vascular concern threshold, however, the method may optionally prompt the user or a third party for an assessment (e.g. physical exam or further investigations). When the magnitude of temperature change does not exceed the vascular concern threshold, method 2000 does not proceed any further, and is instead repeated from step 2010 (e.g. the user vascular concern status continues to be monitored for any changes). The vascular concern threshold may be related to an accepted value indicating a vascular health change (for example but not limited to the following, the vascular concern threshold may be a 0.01 degree Celsius temperature change). Optionally, the method 2000 continues only if the temperature change is determined to be statistically significant.

At steps 2085, optional step 2084 and optional step 2083, the method 2000 then proceeds to compare the temperature trend to a cooling rate threshold. The cooling rate threshold may be related to an accepted vascular or inflammation health change rate. If the temperature trend exceeds the cooling rate threshold, the temperature trend is cooling and the vascular status may be indicative of worsening peripheral vascular disease (i.e. the vascular flow is getting worse over time and the temperature trend reflects a cooling trend) and if an intervention has been applied, the success of the procedure or intervention administered may be deemed to have no impact or not to be effective and the method proceeds to step 2097 (i.e. the success of the procedure or intervention administered may not be confirmed). At optional step 2084, if the temperature trend meets the cooling rate threshold, there may be no vascular or inflammation status change over the time period. In one example, the cooling rate threshold is zero and a null temperature trend may indicate no change in the user's vascular health. In another example, if an intervention has been applied, the temperature trend meeting the cooling rate threshold may indicate that the success of the procedure or intervention administered may not be confirmed and the method proceeds to optional step 2096 (i.e. the success of the intervention administered may not be confirmed or it may be determined to have no impact). Temperature trends may be optionally utilized to evaluate the impact of a vascular concern or vascular intervention on sites distance from the vascular concern. For example, if a vascular concern exists in the right limb, temperature trends may be evaluated in the left limb to understand if the right limb vascular concern is consequently increasing or decreasing the blood flow in other areas. Temperature trends may be utilized to evaluate the impact of a vascular concern as a function of location in the body. In one example, the temperature trend is determined to be more drastic distally in the foot and least drastic at the heel. At optional step 2083, if the temperature trend falls below the cooling rate threshold, the temperature trend is warming and the vascular concern status may be indicative of inflammation or vascular concerns (e.g. the local vascular flow is indicative of a wound or infection and the temperature trend is indicative of a warming trend) and the method proceeds to optional step 2095.

At steps 2095, 2096, or 2097, a vascular concern status flag is generated. The vascular concern status flag may include an alert. The vascular concern status flag may be presented to the user and/or to a third party. A vascular concern status flag may occur in various areas of the foot and the flag may be generated where the vascular concern status is identified. The location of the vascular concern on the user's foot may be identified as a flag on a digital model of a foot, lower limb or other body part. The digital model of the foot may be reviewed by the user and/or a third party. The magnitude of temperature change, temperature trend, and the location of the vascular concern status flag may be indicated on a thermal image display of the foot, lower limb or other body part, on a digital dashboard. In other examples, the vascular concern status flag may be presented as a general alert to the user, without an indication of the location on the foot. The vascular concern status flag may be used to adjust the care actions or care level of the user (i.e. sending a message “our remote monitoring has revealed a consistent trend spanning several months, indicating a gradual cooling of the temperatures measured for the left foot. Multiple factors may contribute to this trend, including seasonal fluctuations, changes in the environment and patient behaviors, as well as changes in underlying peripheral vascular health. We advise confirming that the patient has undergone appropriate peripheral vascular disease screening, in alignment with local guidelines.”). In some examples, the care action may include an intervention such as an input device 100 modification, amputation, an assessment, imaging, diagnostic tests, physical exams, prescription of an assistive device, a vascular procedure, medication, pharmacological therapy, activity/movement prescription, or the cessation or reversal of a care action.

In addition to generating the vascular concern status flag, the flag for vascular status may be indicated to a third party, and the third party may initiate a personalized temperature offset to prevent any further alerting. Alternatively, the personalized temperature offset may be applied as part of step 330 in method 300, step 430 in method 400, step 530 in method 500, step 1830 in method 1800, step 1930 in method 1900, or step 2030 in 2000. A personalized temperature filter may also be calculated to remove irrelevant temperature readings caused by the vascular concern status. The input device 100 may then be used to detect worsening of the vascular concern status and/or additional foot health changes. The third party may initiate a care action after the vascular concern status flag is generated. In other examples, the care action may include an intervention such as an input device 100 modification, amputation, an assessment, imaging, diagnostic tests, physical exams, prescription of an assistive device, a vascular procedure, medication, pharmacological therapy, activity/movement prescription, or the cessation or reversal of a care action.

In further examples, the vascular concern status flag generated may be related to the time period (e.g. a first flag from a sudden drop in the magnitude of temperature change versus a second flag from a progressive drop in the magnitude of temperature change) or related to the magnitude of temperature change (e.g. a third flag from a large drop in temperature over a time period versus a fourth flag in response to the same drop in temperature over a longer time period).

In one example, the input device 100 continues monitoring at least one of posture, activity, or movement of the user by collecting pressure or rate of change of movement measurements at a location on the body of the user. In another example, pressure measurements include at least one pressure sensor or at least one accelerometer disposed in footwear, an insole, a pedometer, a watch, a wearable, a smartphone, or another tracker worn by user.

After steps 2095, 2096 or 2097, the method 2000 may proceed again from step 2010 to continue monitoring of the user's vascular asymmetry status. In other examples, after steps 2095, 2096 or 2097, the method 2000 is followed by methods 300, 400, 500, 600, 700, 800, 900, 1800, or 1900. The continuous monitoring of the user aids in tuning out noise generated by the input system 100 over time.

While the above description provides examples of one or more methods, systems, or devices, it will be appreciated that other methods, systems, or devices may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any user matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A method for detecting a vascular concern, the method comprising:
at each of a series of time points over a time period, collecting a first temperature reading at a first plantar location on a body of a user, to yield a first series of temperature readings;
at each of the series of time points over the time period, collecting a second temperature reading at a second plantar location on the body of the user, to yield a second series of temperature readings, the first series of temperature readings and the second series of temperature readings collected by an input device, the input device worn on a foot of the user;
comparing the first series of temperature readings to the second series of temperature readings to determine a temperature difference for each time point and generating a series of temperature differences;
generating a magnitude of asymmetry from the series of temperature differences; and
generating a vascular concern flag if the magnitude of asymmetry from the series of temperature differences exceeds a vascular concern threshold.

2. The method of claim 1, wherein the first plantar location on the body of the user and the second plantar location on the body of the user are on a same foot of the user.

3. The method of claim 1, wherein the first plantar location on the body of the user corresponds to a first angiosome of the user and the second plantar location on the body of the user corresponds to a second angiosome of the user.

4. The method of claim 1, wherein generating the magnitude of asymmetry from the series of temperature differences comprises determining a measure of central tendency of the temperature differences of the series of temperature differences.

5. The method of claim 1, wherein the vascular concern flag corresponds to a risk level.

6. The method of claim 1, further comprising monitoring at least one of posture, activity, or movement of the user, and the monitoring at least one of posture, activity, or movement of the user comprises collecting pressure or motion measurements at a location on the body of the user.

7. The method of claim 6, wherein monitoring at least one of posture, activity, or movement of the user comprises collecting pressure or motion measurements at a location on the body of the user.

8. The method of claim 1, further comprising
determining a temperature offset;
applying the temperature offset to the first series of temperature readings to generate a first series of offset temperature data;
applying the temperature offset to the second series of temperature readings to generate a second series of offset temperature data; and
identifying and removing irrelevant temperature readings from the first or second series of temperature readings or identifying and removing irrelevant offset temperature data from the first or second series of offset temperature data.

9. The method of claim 1, wherein collecting temperature readings comprises a plurality of the time periods.

10. A system for detecting a vascular concern, the system comprising:
an input device worn on a foot of a user, the input device comprising:
a first temperature sensor located at a first plantar location on a body of the user, the first temperature sensor used to collect a first temperature reading at each of a series of time points over a time period to yield a first series of temperature readings;
a second temperature sensor located at a second plantar location on the body of the user, the second temperature sensor used to collect a second temperature reading at each of the series of time points over the time period to yield a second series of temperature readings;
a processor in communication with the input device, the processor configured to:
receive the first series of temperature readings and the second series of temperature readings from the input device;
compare the first series of temperature readings to the second series of temperature readings to determine a temperature difference for each time point and generating a series of temperature differences;
generate a magnitude of asymmetry from the series of temperature differences; and
generate a vascular concern flag if the magnitude of asymmetry from the series of temperature differences exceeds a vascular concern threshold.

11. The system of claim 10, wherein the first plantar location and the second plantar location are each located under one of a heel, a first metatarsal, a third metatarsal, a fifth metatarsal, and a hallux.

12. The system of claim 10, wherein the first plantar location is on a first foot of the user and the second plantar location is on a second foot of the user.

13. The system of claim 10, wherein the second temperature reading at the second plantar location is an average of temperature readings of all or a subset of plantar locations collected by the input device.

14. The system of claim 10, wherein the vascular concern flag comprises an alert.

15. The system of claim 10, wherein the processor is further configured to:
determine a temperature offset;
apply the temperature offset to the first series of temperature readings to generate a first series of offset temperature data;
apply the temperature offset to the second series of temperature readings to generate a second series of offset temperature data; and
identify and remove irrelevant temperature readings from the first or second series of temperature readings or identifying and removing irrelevant offset temperature data from the first or second series of offset temperature data.

16. The system of claim 15, wherein the temperature offset for the first series of temperature readings and/or the second series of temperature readings is zero.

17. The system of claim 15, wherein the first series of temperature readings and the second series of temperature readings comprise at least one data session, and the at least one data session starts when the user dons the input device and the at least one data session ends when the user doffs the input device.

18. The system of claim 15, wherein determining the temperature offset comprises:
removing the at least one data session if the at least one data session fails to meet a data integrity criterion; and
generating the temperature offset if a minimum number of data sessions is achieved and a calculation period has ended.

19. The system of claim 10, wherein the input device is footwear, an insole, or a pair of insoles.

20. The system of claim 10, wherein the processor is further configured to monitor at least one of posture, activity, or movement of the user.
